(19) United States
(12) Patent Application Publication
(10) Pub. No.: US 2011/0229483 A1
(43) Pub. Date: Sep. 22, 2011

(54) TUMOR ANGIOGENESIS ASSOCIATED GENES AND A METHOD FOR THEIR IDENTIFICATION

(75) Inventors: Arjan Willem Griffioen, Maastricht (NL); Judith Rosina Van Beijnum, Maastricht (NL)

(73) Assignee: Universiteit Maastricht, Maastricht (NL)

(21) Appl. No.: 13/092,778

(22) Filed: Apr. 22, 2011

Related U.S. Application Data
(62) Division of application No. 12/088,670, filed on Mar. 28, 2008, now abandoned.

(30) Foreign Application Priority Data
Sep. 30, 2005 (EP) 05447220.4

(51) Int. Cl. A61K 39/395; A61K 38/16; A61P 35/04; C12Q 1/68; G01N 33/566; C12Q 1/02

(52) U.S. Cl. 424/139.1; 514/19.3; 435/6.11; 435/7.1; 435/29

(57) ABSTRACT

Crucial to designing anti-angiogenic and vascular targeting approaches is the identification of specific target molecules. We compared transcriptional profiles of tumor endothelial cells with that of normal resting endothelial cells, normal but angiogenically activated placental endothelial cells, and cultured endothelial cells. Although the majority of transcripts were classified as general angiogenesis markers, we identified 17 genes that show specific overexpression in tumor endothelium. Antibody targeting of four cell-surface expressed or secreted products (vimentin, CD59, HMGB1 and IGFBP7) inhibited angiogenesis in vitro and in vivo. Finally, targeting endothelial vimentin in a mouse tumor model significantly inhibited tumor growth and reduced microvessel density. Our results demonstrate the utility of the identification and subsequent targeting of specific tumor endothelial markers for anticancer therapy.

### BACKGROUND OF THE INVENTION

Tumor progression and the development of distant metastases require the presence of an extensive vasculature. Active angiogenesis is a hallmark of most malignancies and inhibition of this process is considered to be a promising strategy for the treatment of tumors. In order to develop the most specific and effective anti-angiogenic therapies for treating cancer, it is of importance to have a fundamental understanding of the molecular differences between tumor endothelial cells and their normal counterparts. Since angiogenesis is not limited to pathological conditions, careful evaluation of the putative targets is necessitated to prevent side effects associated with impaired physiological angiogenesis.

### Angiogenesis

Angiogenesis occurs in the healthy body for healing wounds and for restoring blood flow to tissues after injury. In females, angiogenesis also occurs during the monthly reproductive cycle, e.g. to rebuild the uterus lining and to mature the egg during ovulation, and during pregnancy, e.g. to build the placenta and the circulation between mother and fetus. The healthy body controls angiogenesis through a series of angiogenesis-stimulating growth factors and angiogenesis inhibitors. When angiogenic growth factors are produced in excess of angiogenesis inhibitors, the balance is tipped favor of blood vessel growth. When inhibitors are present in excess of stimulators, angiogenesis is stopped. The normal, healthy body maintains a perfect balance of angiogenesis modulators. In general, angiogenesis is “turned off” when more inhibitors being produced than stimulators. In general it is believed that tumors produce large amounts of angiogenic growth factors, overwhelming natural inhibitors, to recruit their own blood supply.

Angiogenesis not only allows solid tumors to grow, it also makes them more dangerous because they are more likely to metastasize, i.e. spread elsewhere in the body through the bloodstream. The new blood vessels in the tumor increase the chance of cancer cells getting into the blood, especially since the tumor's blood vessels are often imperfectly formed. Moreover, it is reported that human breast cancers which became metastatic had many more blood vessels than those which did not. In order to grow larger than about two cubic millimetres, metastases require their own system of newly formed blood vessels. It is believed that if you could stop the said vascularization, it would be possible to cut the supply line to primary tumors as well as the tumor's metastases, causing them to starve.

### Gene Expression

Gene expression profiling techniques are widely used to detect changes in transcript expression levels and provide the tools to study molecular events in biological processes or to identify tissue or tumor endothelial specific markers. Different cell culture models have been developed to study angiogenesis, but the temporal and spatial complex actions of all factors exerting effect on endothelial cells in vivo may not be accurately reflected in vitro. Gene expression analysis of tumor endothelial cells (TECs) encounters difficulties related to the fact that endothelial cells (ECs) are embedded in complex tissues and comprise only a small fraction of the cells present in these tissues.

### Cultured Endothelial Cells

Several laboratories have reported gene expression profiles of cultured endothelial cells that were subject to pro-angiogenic growth factor stimulation. In cell culture conditions, however, cells reside in an artificial microenvironment and might respond aberrantly to certain stimuli, giving a false representation of the in vivo situation. In fact, genes induced in these studies are highly biased to metabolic function, protein turnover and cell turnover (Abe and Sato, 2001; Dell'Era et at, 2002; Gerritsen et al., 2003b; Van Beijnum and Griffioen, 2005; Wang et al., 2003; Zhang et al., 1999). This “cell-cycle signature” can be related to the transition from quiescent to proliferative endothelium, which is an early event in angiogenesis.

An alternative in vitro approach uses the three-dimensional culture of endothelial cells in matrix components such as collagen. Endothelial cell tube formation in vitro is mainly associated with changes in the expression of genes that mediate cell-cell contact and cell-matrix interactions, such as adhesion molecules and matrix metalloproteinases (Van Beijnum and Griffioen, 2005). Nevertheless, the complex microenvironment of angiogenic endothelial cells in tissues is extremely difficult to mimic adequately in vitro. In addition, when regarding angiogenesis in cancer, tumor endothelial cells have resided in the tumor microenvironments for months to years, whereas culture systems only cover a time period of days, which in addition contributes to discrepancies in observed gene expression profiles of endothelial cells in vitro vs in vivo.

In conclusion, it appears difficult to accurately mimic in vitro the complex temporal and spatial actions of all microenvironmental factors exerting an effect on endothelial cells in vivo. Therefore, extrapolation of data generated by in vitro experiments to the in vivo situation is limited, stressing the importance of approaches that make use of more relevant cell sources such as tissue derived cells.

### Freshly Isolated Tumor ECs

To date, only a limited number of studies have characterized the gene expression profile of freshly isolated tumor ECs (Madden et al., 2004; Parker et al., 2004; St Croix et al., 2000). For instance, SAGE tag repertoires were generated from ECs isolated from both tumor and normal tissues and compared to identify differentially expressed genes. Notably, an extensive bias towards genes functioning in extracellular matrix remodelling among the Tumor endothelial markers (TEMs) was evident in published SAGE data sets of isolated tumor endothelial cells that were compared to, solely, normal endothelial cells (Parker et al., 2004; St Croix et al., 2000). The same was true for glioma endothelial markers (GEMs) (Madden et al., 2004). Apparently, genes thought to play a role in the initiation of angiogenesis are only rarely identified in gene expression profiling of endothelial cells derived from tumors (Madden et al., 2004; Parker et al., 2004; St Croix et al., 2000).

In these studies using freshly isolated tumor ECs, however, gene expression associated with physiological processes never was taken into account.

### SUMMARY OF THE INVENTION

A most crucial element in designing anti-angiogenic and vascular targeting approaches is the identification of specific target molecules.

Although it appears that some tumor endothelial cell associated markers have been identified, translation to the clinic remains a hurdle to be taken, predominantly since the prior art TAG-like molecules were not evaluated vis a vis physiological angiogenesis (TAG=tumor angiogenesis associated gene).

In the present study, the identification of markers of tumor EC is described that are also overexpressed as compared to physiologically activated placenta EC. Specifically, gene expression profiles of isolated EC from malignant colon carcinoma tissues, non-malignant angiogenic placenta tissues, as well as from non-angiogenic normal resting tissues were evaluated by using suppression subtractive hybridisation (SSH). In addition, these gene expression profiles were compared with an in vitro model of tumor-conditioned EC activation.

A large overlap in the expression of markers of tumor endothelium and physiologically angiogenic endothelium was observed. Hence, the present invention demonstrated that gene expression profiles of tumor derived and placenta derived endothelial cells reflect the later stages of angiogenesis, and though most upregulated genes are representative of physiological angiogenesis, a number of genes contribute specifically to a tumor endothelium specific phenotype. In addition, it was shown that in vitro EC activation is only to a very limited extent representative of tumor angiogenesis.

In the present invention, 17 genes were identified in detail that were specifically overexpressed in tumor endothelium, among which a number of genes coding for surface expressed or secreted protein products, e.g., vimentin, CD59, HMGB1 and IGFBP7. Antibodies targeting these proteins inhibited angiogenesis both in in vitro and in vivo assays. Targeting endothelial proteins in tumor models significantly and dose-dependently inhibited tumor growth and reduced microvessel density, with minimal effects on physiological angiogenesis.

This is the first report to investigate gene expression in endothelial cells, in which a direct distinction is made between pathological and physiological angiogenesis in comparison with quiescent endothelium, using both in vitro and in vivo sources. These findings have crucial impact on the design and improvement of angiogenesis interfering strategies for treatment of human disease.

### DETAILED DESCRIPTION OF THE INVENTION

Active angiogenesis is a hallmark of most malignancies and processes in which tissue growth is essential. Identification of tumor angiogenesis associated genes (TAGs) within the present invention, provides primary targets for the development of molecular imaging approaches and therapeutic modalities for combating cancer. However, the present invention also contemplates the identification of (general and/or specific) angiogenesis associated genes which, in addition, may provide targets for other angiogenesis dependent proliferative diseases. The present invention thus provides a method for identifying tumor angiogenesis associated genes, wherein said tumor is primarily malignant (cancer) but may also be benign.

The term “cancer” within the present specification refers to any disease characterized by uncontrolled cell division leading to a malignant (cancerous) tumor (or neoplasm, abnormal growth of tissue). Malignant tumors can invade other organs, spread to distant locations (metastasize) and become life threatening. The term “proliferative disease” refers to the rapid proliferation of cells which may either lead to a benign (not cancerous) tumor (or neoplasm) that does not spread to other parts of the body or invades other tissues—they are rarely a threat to life, or which may lead to a malignant (cancerous) tumor (or neoplasm).

Since angiogenesis is not limited to pathologies or disease, careful evaluation of putative therapeutic targets is necessary to prevent side effects associated with impaired physiological angiogenesis. In contrast to the prior art, within the present invention, transcriptional profiles of angiogenic endothelial cells isolated from both malignant and non-malignant tissues were compared with resting endothelial cells to identify tumor-specific angiogenesis markers and to distinguish these from general angiogenesis markers. Targeting TAG proteins with antibodies inhibited angiogenesis in vitro and in vivo, confirming their active contribution to the process and confirms therapeutic applications. Accordingly, the present invention relates to a method for identifying tumor angiogenesis associated genes, and the use thereof in diagnosis, therapy, and identification of modulators of angiogenesis.

### A. GENERAL TECHNIQUES

The practice of the present invention will employ, unless otherwise indicated, conventional techniques of organic chemistry, pharmacology, molecular biology (including recombinant techniques), cell biology, biochemistry, and immunology, which are within the skill of the art.

Such techniques are explained fully in the literature, such as, “Molecular Cloning: A Laboratory Manual”, Second Edition (Sambrook et al., 1989); “Oligonucleotide Synthesis” (M. J. Gait, ed., 1984); “Animal Cell Culture” (R. I. Freshney, ed., 1987); the series “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987, and periodicals); “Polymerase Chain Reaction” (Mullis et al., eds., 1994); and “Current Protocols in Immunology” (J. E. Coligan et al., eds., 1991).

### B. DEFINITIONS

As used herein, certain terms may have the following defined meanings. As used in the specification and claims, the singular form “a,” “an” and “the” include plural references unless the context clearly dictates otherwise. For example, the term “a cell” includes a plurality of cells, including mixtures thereof. Similarly, use of “a compound” for treatment or preparation of medicaments as described herein contemplates using one or more compounds of this invention for such treatment or preparation unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like.

“Consisting of” shall mean excluding more than trace elements of other ingredients and substantial method steps for administering the compositions of this invention.

Throughout this disclosure, various publications, patents and published patent specifications are referenced by an identifying citation. The disclosures of these publications, patents and published patent specifications are hereby incorporated by reference into the present disclosure to describe more fully the state of the art to which this invention pertains.

Embodiments defined by each of these transition terms are within the scope of this invention.

### C. METHOD FOR IDENTIFYING TAGs

The present invention relates particularly to a method for identifying specific tumor angiogenesis associated genes (TAGs). In contrast to the prior art, the present invention compared the expression profiles of tumor endothelial cells with resting endothelial cells from normal tissue, endothelial cells from placenta and cultured resting and stimulated endothelial cells.

The term “expression profiles” is well known in the art, and relates to the determination of spatial and temporal expression of genes. In particular, expression profiling may include determining the spatial and temporal amount of mRNAs, relative to metabolic conditions, genotypes, or physiopathological states of analysed tissues, and subsequent bioinformatics analysis, to characterize gene involvement in angiogenesis. Expression profiling may help elucidating what genes show different expression levels in different samples; elucidating the patterns of expression of the genes; elucidating the function of a particular gene; and elucidating the relationship with other information about these genes. The person skilled in the art is knowledgeable about algorithms and tools of bioinformatics used in expression profiling.

Techniques to differentiate between expression in different tissues are well known in the art, and include techniques such as SAGE, Suppression Subtractive Hybridization (SSH), differential display, microarray analysis, and oligonucleotide array analysis (e.g., Affymetrix).

SSH is a subtraction technique, creating a cDNA repertoire of sequences overexpressed in one tester cDNA population compared to the other driver cDNA population.

Compared to SAGE, SSH is much less labour intensive on a technical as well as a logistic level. Furthermore, unlike SAGE, SSH provides cDNA repertoires comprising individual partial cDNAs having a characteristic overexpression in tester compared to driver cDNA. These individual cDNAs may then be used as a starting point for multiple purposes including their use in immobilisation of target molecules in cDNA arrays, use as labelled probes for hybridisation experiments such as e.g., Northern blotting etc, use in the expression of partial proteins in functional studies, and use as template molecule for generating siRNA.

SSH consists of 2 hybridization steps, followed by suppression PCR to reduce the redundancy of overexpressed cDNAs. In a first step, 2 tester cDNA populations—ligated to different adaptor sequences—are hybridized in separate reactions to an excess of driver cDNA to subtract common sequences in tester and driver cDNA populations i.e. the non-differentially expressed genes. The cDNA is amplified, (using for instance Clontech SMART™ cDNA amplification kit) generating sufficient starting material for tester and driver, whereby the original transcript distribution is maintained. In the second hybridization the two primary hybridization samples are mixed and here create the template for the subsequent suppression PCR. During this reaction, inverted terminal repeats prevent amplification of highly abundant molecules and the amplification of differentially expressed genes is favoured. The final cDNA repertoire generated by PCR consists of cDNA fragments that are overexpressed in the tester as compared to the driver population (Clontech protocol # PT1117-1). Amplification of the target genes is dependent upon the template, such as the length, the GC content, and/or presence of inverted repeats. Therefore, the number of amplification cycles in either step is of crucial importance. As such, the method of the present invention limits the number of cycles, and preferably adapts the number of cycles to e.g. 2, 3, 4, 5, 6, 7, 8, 9 or 10 cycles, in the amplification step(s) of the SSH to ensure effective subtraction and suppression. Optimization of the number of amplification cycles ensures proper suppression and reduction of redundancy. The person skilled in the art may use routine trial and error to establish the optimum or near-optimum number of cycles to satisfy the specific needs, e.g. the provision of the original representation of transcripts.

Another main advantage of using SSH is that it is independent on previously cloned genes. Although the DNA microarray technique is considered one of the keys for deciphering the information content of the genome, i.e. measuring the expression levels of single genes or thousands of genes simultaneously, microarray technologies have an extremely important drawback in that only previously known and cloned genes are considered, e.g. commercially available array systems are available with gene sets biased to a particular disease. Depending on the research question and experimental setup this gene set may or may not be relevant to screen for differentially expressed genes. However, since the density of DNA sequences on a given glass slides has limitations, certain genes will not be represented.

Nevertheless, microarrays can be custom-made, and the commercially available array systems aim to cover more and more of the (human) expressed genome. As such, microarrays may find their use in combination with SSH. SSH is biased to genes of interest in the experimental setup because of the subtraction and suppression. Since it will not always be 100% effective and does not give direct information about the extent of overexpression, it may be advisable to perform cDNA array screening of the SSH repertoires.

In a first embodiment, the present invention provides a method for identifying specific tumor angiogenesis associated genes (TAGs), said method comprising:
(a) producing a cDNA library from tumor endothelial cells (TEC), producing a cDNA library from normal endothelial cells (NEC), and producing a cDNA library from active endothelial cells (AEC),
(b) performing suppression subtractive hybridisation (SSH) of TEC subtracted with NEC and TEC subtracted with AEC, and
(c) identifying the cDNAs that are overexpressed in TEC relative to NEC and/or AEC as TAGs.

In a second embodiment, the present invention provides a method for identifying specific tumor angiogenesis associated genes (TAGs), said method comprising:
(a) producing a cDNA library from tumor endothelial cells (TEC), producing a cDNA library from normal endothelial cells (NEC), and producing a cDNA library from active endothelial cells (AEC),
(b) performing suppression subtractive hybridisation (SSH) of NEC subtracted with TEC and AEC subtracted with TEC, and
(c) identifying the cDNAs that are underexpressed in TEC relative to NEC and/or AEC as TAGs.

The term “endothelial cell” is well-known to the person skilled in the art and relates to a thin, flattened cell, of which a layer lines the inside surfaces of e.g., body cavities, blood vessels, and lymph vessels, making up the endothelium. Endothelial cells perform several functions, including acting as a selective barrier to the passage of molecules and cells between the blood and the surrounding bodily tissue; they play an essential role in summoning and capturing white blood cells (leukocytes) to the site of an infection; they regulate coagulation of the blood at the site of a trauma; they regulate the growth of the vascular muscular cells; and they secrete and modify several vascular signaling molecules. A “normal endothelial cell” relates to a resting or quiescent endothelial cell, i.e. an endothelial cell which is not activated to an angiogenic state. As used herein, “active endothelial cell” (AEC) relates to endothelial cells which are activated to an angiogenic state, such as tissues with enhanced angiogenesis but which are not related to malignant tissues, such as, for instance, endothelial cells involved in the female productive processes and revascularization in wound healing (i.e., physiological angiogenesis). Accordingly, AEC includes, but is not limited to “placental endothelial cell” (PLEC). PLEC relates to endothelial cell derived from a placenta. In the present invention, the term “tumor endothelial cell” (TEC) relates to an endothelial cell which is activated to an angiogenic state, and which is related to a malignant tissue. For instance, TECs can be derived from colorectal tumor endothelial cells or tumorigenic endothelial cells induced by malignant gliomas, e.g., glioma-endothelial cells (GECs). For comparison purposes, and evaluating the similarity with endothelial cells derived from tissues, cultured endothelial cells can be used. In this regard, human umbilical vein endothelial cells (HUVEC) are ubiquitously used. HUVECs can be freshly isolated and cultured for one or a few passages. Alternatively, established HUVEC cell lines can be used, such as the EC line EVLC2, which is a cell line derived from human umbilical vein ECs by immortalization with simian virus 40 large T antigen (Leeuwen et al., 2001). Cultured endothelial cells can be activated by agents to differentiate, migrate, etc (HUVEC+). These activated cultured endothelial cells are preferably used to identify differential expression patterns resulting from activation by a particular agent, such as TPA (12-O-tetra-decanoylphorbol-13-acetate). Within the present invention, the term “HUVEC+” refers to activated cultured HUVEC cells. The term “HUVEC−” refers to non-activated, quiescent, cultured or primary HUVEC cells.

The terms “angiogenesis” and “activated to an angiogenic state” are well known in the art and relate to the formation of new branches from pre-existing blood vessels. Angiogenesis occurs, for instance, in the female reproductive tract during the formation of the corpus luteum, during endometrial development and during embryo implantation and placentation. This type of vessel growth also occurs during pathologic conditions, such as retinopathies, arthropathies, wound healing, tumor growth and metastases.

It is believed that in wound healing, hypoxic macrophages release angiogenic substances at the edges or outer surfaces of wounds that initiate revascularization. Solid tumors require their own system of newly formed blood vessels in order to grow larger than about two cubic millimeters. Beyond the critical volume of 2 cubic millimeters, oxygen and nutrients have difficulty diffusing to the cells in the center of the tumor, causing a state of cellular hypoxia that marks the onset of tumoral angiogenesis. In addition, tumors need vasculature to dispose of their metabolic waste products. New blood vessel development is an important process in tumor progression. It favors the transition from hyperplasia to neoplasia i.e. the passage from a state of cellular multiplication to a state of uncontrolled proliferation characteristic of tumor cells. Neovascularization also influences the dissemination of cancer cells throughout the entire body eventually leading to metastasis formation. The vascularization level of a solid tumor is thought to be an indicator of its metastatic potential.

The TAGs identifiable by the method of the invention are over- or under-expressed in tumor endothelial cells relative to normal endothelial cells and/or AEC, such as PLEC.

In order to obtain generally useful TAGs and to rule out individual expression differences, the method according to the invention preferably evades source related differences. As such, the method may incorporate patient matched endothelial cells derived from normal tissue (NEC), and malignant tissue (TEC). The advantage of patient-matched endothelial cells as described above is that artifacts in TEC and NEC are allowed to be leveled out against one another. In order to further evade individual expression differences or MHC (Major Histocompatibility Complex) class differences, the endothelial cells for producing cDNA libraries may be pooled from at least two different patients, and preferably from more patients, such as, for instance, from 3, 4, 5 or even more patients.

The endothelial cells are embedded in other cell types, which obscure endothelial cell specific expression. Therefore, in a further embodiment, the present invention relates to endothelial cells for producing cDNA libraries which are isolated to at least 90% purity, and preferably, to at least 95%, 96%, 97%, 98%, 99% or 100% purity. Methods for purifying cells are known in the art, and are described for instance in the examples section. For instance, endothelial cells may be isolated by using endothelial specific cell markers, e.g. CD31 and/or antibodies directed thereto, e.g. anti-CD31, and cell sorting, e.g. FACS.

The term “purified” as applied herein, refers to a composition wherein the desired component, such as a polypeptide, nucleic acid, antibody, cell, etc comprises at least 50%, 60%, 70%, 80%, 90% and preferably at least 95%, 96%, 97%, 98%, 99% or 100% of the desired component in the composition. The composition may contain other compounds, such as carbohydrates, salts, solvents, lipids, and the like, without affecting the determination of percentage purity as used herein.

### D. ISOLATED TARGETS

The method according to the invention allows the identification of differentially expressed genes by pair-wise comparisons, such as in TEC relative to AEC, or NEC, or cultured endothelial cells (freshly isolated and cultured EC or established EC cell lines). Specifically, the present invention relates to the identification of genes which are over- or under-expressed in TEC relative to NEC; TEC relative to AEC, such as PLEC; AEC such as PLEC relative to NEC; and HUVEC+ relative to HUVEC−. In particular, the present invention relates to the identification of differentially expressed genes in TEC relative to AEC, such as PLEC, and TEC relative to NEC.

### D1. TAGs

Accordingly, the present invention relates to tumor angiogenesis associated genes (TAGs) identifiable by the method of the invention (Table 2). The group of TAGs includes the nucleic acids as depicted in Table 2, i.e. characterized by the GenBank accession numbers: NM_{—}152862.1, NM_{—}000611, NM_{—}004642.2, NM_{—}000088.2, NM_{—}001845.2, NM_{—}002128.3, BC003378, BC041913, NM_{—}014571, NM_{—}017994.1, X02160, NM_{—}001553, NM_{—}002300, CV337080, AJ320486, NM_{—}003118.1, AF077200 and X56134, which are included herein specifically by reference. Particularly, the present invention relates to isolated polynucleotides comprising or consisting of nucleic acids characterized by any of SEQ ID NO:s 1, 3, 5, 7, 9, 11, 13, 15, 17, 19, 21, 23, 25, 27, 29, 31 or 33, or a part thereof, the complement thereof, or a variant of said nucleic acids. It will be appreciated that the present invention also relates to parts and complements of said variants. The connoted parts are preferably unique parts (i.e., non-repetitive sequence parts and/or not present in other genes). Using routine techniques, the person skilled in the art is able to establish the percentage identity. The present invention is also directed to variants of the nucleotide sequence of the nucleic acid disclosed in the invention, and preferably any of SEQ ID NO:s 1, 3, 5, 7, 9, 11, 13, 15, 17, 19, 21, 23, 25, 27, 29, 31 or 33, or the corresponding complementary strand.

The nucleotide and amino acid sequences of the TAG genes of the invention are depicted in FIG. 9A. The nucleotide sequences of SSH identified TAG inserts are depicted in FIG. 9B.

The term “variant” relates to a nucleic acid molecule which is at least 65%, 70%, 75%, 80%, 85%, 90%, 95%, 96%, 97%, 97.5%, 98%, 98.5%, 99% or 99.5% identical to the nucleotide sequences of the invention, and preferably as represented in SEQ ID NO:s 1, 3, 5, 7, 9, 11, 13, 15, 17, 19, 21, 23, 25, 27, 29, 31 or 33, or the corresponding complementary strand, or parts thereof.

By a nucleic acid having a nucleotide sequence of at least, for example, 95% “identity” to a reference nucleotide sequence of the present invention, it is intended that the nucleotide sequence of said nucleic acid is identical to the reference sequence except that the nucleotide sequence may include up to five point mutations per each 100 nucleotides of the reference nucleotide sequence. In other words, to obtain a nucleic acid having a nucleotide sequence of at least 95% identity to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. As a practical matter, whether any particular nucleic acid molecule is at least 65%, 70%, 75%, 80%, 85%, 90%, 95%, 96%, 97%, 97.5%, 98%, 98.5%, 99% or 99.5% identical to a nucleotide sequence of the present invention can be determined using known algorithms. A preferred method for determining the best overall match between a query sequence (a sequence of the present invention) and a subject sequence can be determined using a Blast search (Altschul et al., 1997). It will be appreciated that the terms “nucleic acids” and “nucleotide sequence” are used interchangeably herein.

By using the method of the invention, it may be possible that, inherent to molecular biology techniques, only part of the transcript corresponding to the differentially expressed gene is isolated. Nevertheless, even part of the transcript, and also the corresponding cDNA, allows determining the identity of the gene. For instance, after establishing the sequence of the (partial) transcript or cDNA, the identity of the corresponding gene can be established by a sequence comparison with commonly available sequences, such as present in the GenBank. Alternatively, in case the corresponding gene is not known, the complete sequence of the gene can be revealed by routine molecular biological techniques, such as for instance screening cDNA libraries, preferably derived from endothelial cells, including but not limited to endothelial tumor tissue such as malignant endothelial cell derived tumors e.g. angiomas, and gene-walking. Accordingly, the nucleotide sequences presented in the present invention may be extended starting from a partial nucleotide sequence and employing various methods known in the art to detect the full sequence in case said sequence would only be a part of a coding region as well as upstream sequences such as promoters and regulatory elements.

The identification of the differentially expressed genes by the method according to the invention facilitates the identification of the corresponding amino acid sequence. Accordingly, the present invention relates to isolated polypeptides comprising, or alternatively consisting of, an amino acid sequence according to the invention, and preferably characterized by any of SEQ ID NO:s 2, 4, 6, 8, 10, 12, 14, 16, 18, 20, 22, 24, 26, 28, 30, 32 or 34, or a part thereof, or comprising or consisting of a variant thereof, or an immunologically active and/or functional fragment thereof.

A variant peptide is characterized by an amino acid sequence which is at least 65%, 70%, 75%, 80%, 85%, 90%, 95%, 96%, 97%, 97.5%, 98%, 98.5%, 99%, 99.5% or 100% identical to an amino acid sequence according to the invention, and preferably characterized by any of SEQ ID NO:s 2, 4, 6, 8, 10, 12, 14, 16, 18, 20, 22, 24, 26, 28, 30, 32 or 34, or a part thereof. Using routine techniques, the person skilled in the art is able to establish the percentage identity.

It will be appreciated that the present invention relates to an isolated polypeptide encodable by a nucleic acid according to the invention, or a variant or a derivative thereof, or an immunologically active and/or functional fragment thereof. More preferably, a polypeptide comprising or consisting of an amino acid sequence as given in SEQ ID NO:s 2, 4, 6, 8, 10, 12, 14, 16, 18, 20, 22, 24, 26, 28, 30, 32 or 34, or a variant or a derivative thereof, or an immunologically active and/or functional fragment thereof. Specifically the present invention relates to an isolated nucleic acid comprising a member selected from a group of nucleic acids identifiable as a tumor angiogenesis associated gene (TAG) according to the method of the invention, said group consisting of:
(a) a nucleic acid comprising a DNA sequence as given in SEQ ID NO 1, 3, 5, 7, 9, 11, 13, 15, 17, 19, 21, 23, 25, 27, 29, 31 or 33, or a part thereof, or the complement thereof,
(b) a nucleic acid comprising the RNA sequences corresponding to SEQ ID NO 1, 3, 5, 7, 9, 11, 13, 15, 17, 19, 21, 23, 25, 27, 29, 31 or 33, or a part thereof, or the complement thereof,
(c) a nucleic acid specifically hybridizing to the nucleotide sequence as defined in (a) or (b),
(d) a nucleic acid comprising of a nucleotide sequence, which is at least 65% identical to the sequence defined in (a),
(e) a nucleic acid encoding a protein with an amino acid sequence, which is at least 65% identical to the amino acid sequence as given in SEQ ID NO 2, 4, 6, 8, 10, 12, 14, 16, 18, 20, 22, 24, 26, 28, 30, 32 or 34, or a part thereof,
(f) a nucleic acid encoding a protein comprising the amino acid sequence as given in any of SEQ ID NO 2, 4, 6, 8, 10, 12, 14, 16, 18, 20, 22, 24, 26, 28, 30, 32 or 34, or a part thereof,
(g) a nucleic acid which is degenerated as a result of the genetic code to a nucleotide sequence of a nucleic acid as given in SEQ ID NO 1, 3, 5, 7, 9, 11, 13, 15, 17, 19, 21, 23, 25, 27, 29, 31 or 33, or a part thereof or as defined in (a) to (f),
(h) a nucleic acid which is diverging due to the differences in codon usage between the organisms to a nucleotide sequence encoding a protein as given in SEQ ID NO 2, 4, 6, 8, 10, 12, 14, 16, 18, 20, 22, 24, 26, 28, 30, 32 or 34 or as defined in (a) to (g),
(i) a nucleic acid which is diverging due to the differences between alleles encoding a protein as given in SEQ ID NO 2, 4, 6, 8, 10, 12, 14, 16, 18, 20, 22, 24, 26, 28, 30, 32 or 34, or as defined in (a) to (h),
(j) a nucleic acid encoding an immunologically active and/or functional fragment of a protein encoded by a DNA sequence as given in SEQ ID NO 1, 3, 5, 7, 9, 11, 13, 15, 17, 19, 21, 23, 25, 27, 29, 31 or 33,
(k) a nucleic acid encoding a gene family member of the nucleic acid as given in SEQ ID NO 1, 3, 5, 7, 9, 11, 13, 15, 17, 19, 21, 23, 25, 27, 29, 31 or 33, and,
(l) a nucleic acid encoding a protein as defined in SEQ ID NO 2, 4, 6, 8, 10, 12, 14, 16, 18, 20, 22, 24, 26, 28, 30, 32 or 34, or a nucleic acid as defined in any one of (a) to (k) characterized in that said sequence is DNA, cDNA, genomic DNA or synthetic DNA.

In the present invention, the term “immunologically active” fragment relates to a fragment of the polypeptide according to the invention which comprises an epitope (T-cell and/or B-cell epitope). The minimal length of an epitope will be about 5 amino acids, but is preferably longer, such as, for instance, 6, 7, 8, 9, 10 or even more amino acids.

In the present invention, the term “functional fragment” relates to a fragment of the polypeptide according to the invention, and said functional fragment comprises still at least 60% activity of the protein from which it is derived. The activity of a protein may be determined by functional assays applicable to the particular protein at issue and well known in the art.

### D2. GAGs

In contrast to the prior art, the present invention was able to distinguish between differential expression of genes upregulated and downregulated in TEC compared to the expression of genes involved in NEC and physiological angiogenesis such as female reproductive processes (PLEC) and wound healing, by comparison of the expression patterns of tumor endothelial cells; normal, i.e. resting, endothelial cells; and active but non-malignant endothelial cells. As such, the present invention relates to the identification of differentially expressed genes in physiological angiogenesis of AEC, and preferably PLEC, relative to NEC. Even more preferably, the present invention relates to the identification of differentially expressed genes in AEC, such as PLEC relative to NEC, and TEC relative to NEC (defined as general angiogenesis genes A or GAG/A). Hence, the present invention relates to an isolated nucleic acid comprising a member selected from a group of nucleic acids identifiable as general angiogenesis genes GAG/A according to the method of the invention, or a part thereof, or comprising or consisting of a variant thereof, or an immunologically active and/or functional fragment thereof. The group of GAG/A includes the nucleic acids as depicted in Table 3, i.e. characterized by the GenBank accession numbers: NM_{—}007200, NM_{—}001575, NM_{—}147783.1, NM_{—}005348, NM_{—}001753, BX115183, NM_{—}001921.1, NM_{—}001344, NM_{—}006304, BC047664, NM_{—}007036, AW269823, NM_{—}003107, NM_{—}004280.2, NM_{—}000801, AK056761, BC003394, NM_{—}145058, NM_{—}002211, NM_{—}006479, NM_{—}170705.1, BC011818, NM_{—}033480, NM_{—}032186, NM_{—}002421, NM_{—}002425, NM_{—}001416, BCO25278, NM_{—}014959, M15887, AI793182, BCO32350, NM_{—}002982, NM_{—}002422, NM_{—}021109, BC018163, AA296386, NM_{—}003347, AI422919, NM_{—}004339.2, BCO50637, AY117690.1, NM_{—}015987.2, AK094809.1, NM_{—}000983 and NM_{—}175862, which are included herein specifically by reference.

In order to determine the usefulness of cultured cells in resembling in vivo processes, the expression profiles of cultured endothelial cells, possibly treated with tumor promoting agents and/or agents that activate angiogenesis, may be compared with the expression profiles of AEC, such as PLEC; NEC; and/or TEC. Accordingly, the present invention relates to the identification of differentially expressed genes, such as overexpressed genes, in tumor conditioned HUVEC+ relative to AEC, NEC, and/or TEC. Even more preferably, the present invention relates to the identification of differentially expressed genes in TEC relative to NEC and HUVEC+ relative to HUVEC− (defined as general angiogenesis genes B or GAG/B). As such, the present invention relates to an isolated nucleic acid comprising a member selected from a group of nucleic acids identifiable as general angiogenesis genes B (GAG/B) according to the method of the invention, or a part thereof, or comprising or consisting of a variant thereof, or an immunologically active and/or functional fragment thereof. The group of GAG/B includes the nucleic acids as depicted in Table 3, i.e. characterized by the GenBank accession numbers: NM_{—}001575, NM_{—}005348, BX115183, NM_{—}006304, BC047664, NM_{—}007036, NM_{—}003107, NM_{—}004280.2, BC003394, BC011818, NM_{—}033480, NM_{—}032186, NM_{—}002425, BCO25278, NM_{—}014959, M15887, NM_{—}021109, NM_{—}003347, NM_{—}000442.2 and NM_{—}000982.2, which are included herein specifically by reference.

It will be appreciated that there is an overlap between GAG/A and GAG/B.

### D3. General

The present invention relates also to a nucleic acid molecule of at least 12, or more preferably 13, 14, 15, 16, 17, 18, 19, 20, 25, 30, 40, 50 or even more nucleotides in length specifically hybridizing with a nucleic acid according to the invention. Longer nucleotides are also contemplated, e.g. of about 75, 100, 200 or even more nucleotides. Different types of hybridisation techniques and formats are well known in the art. The said nucleic acid molecule may be labeled, thereby allowing the detection of the hybrid. In this regard, the present invention provides methods for detecting the nucleic acids of the present invention. The term “label” as used in present specification refers to a molecule propagating a signal to aid in detection and quantification. Said signal may be detected either visually (e.g., because it has color, or generates a colored product, or emits fluorescence) or by use of a detector that detects properties of the reporter molecule (e.g., radioactivity, magnetic field, etc.). Labeling systems are well known in the art and include, without limitation, the use of a variety of stains or the incorporation of fluorescent, luminescent, radioactive or otherwise chemically modified nucleotides such as e.g., labeled streptavidin conjugate, digoxigenin, anti-digoxigenin, luciferase, P-galactosidase, antigens, enzymes and enzyme conjugates, (e.g. horseradish peroxidase, alkaline phosphatase and others).

In a further embodiment, the present invention relates to an amplification primer, preferably a nucleic acid molecule of at least 12, or more preferably 13, 14, 15, 16, 17, 18, 19, 20, 25 or even more nucleotides in length specifically amplifying a nucleic acid according to the invention. As such, the nucleic acid is liable to act as a primer for specifically amplifying a nucleic acid of the present invention, or a part thereof.

The primers may be used in any well described amplification technique known in the art such as, for instance, Polymerase Chain Reaction (PCR), TMA (transcripition mediated amplification) or NASBA (nucleic acid sequence based amplification) techniques, thereby allowing the amplification and subsequent detection of the nucleic acid of the present invention. Preferably, said primers may also be used to specifically amplify the nucleic acids of the present invention. As such, the present invention provides methods for detecting the nucleic acids of the present invention.

The primers of the invention provide for specifically amplifying the target sequence. In the present invention, the term “specifically amplifying” relates to the preferred amplification of the target sequence, while non-target sequences are not or less well amplified, because of which the ratio between target sequence versus the non-target sequence is increased. Hybridisation conditions for the primer binding to the target sequence are at least co-decisive for specifically amplifying. In other words, temperature, salt concentration, etc., determine the hybridisation specificity.

Preferably, the present invention provides the amplification primers for TAGs as depicted in Table 4, i.e. SEQ ID NO:s 75-108.

It will be appreciated by the person skilled in the art that the term “specifically” within the context of “specifically hybridising” and “specifically amplifying” relates to the stringent hybridisation of a nucleic acid with a target sequence. It is clear to the skilled person that a specific hybridisation event, in case of an amplification primer, results in a specific amplification.

Nucleic acids which specifically hybridise to any of the strands of the nucleic acid molecules of the present invention, such as characterized by SEQ ID NO:s 1, 3, 5, 7, 9, 11, 13, 15, 17, 19, 21, 23, 25, 27, 29, 31 or 33 under stringent hybridisation conditions or lower stringency conditions are also particularly encompassed by the present invention.

“Stringent hybridisation conditions” are dependent upon the composition of the probe, including length and GC-content, and can be determined by appropriate computer programmes. Hybridisation under high and low stringency conditions are principles which are well understood by the person skilled in the art (see, for instance, Sambrook et al. Molecular Cloning: A laboratory manual. Cold Spring Harbor laboratory press 1989). For instance, in hybridisation experiments, stringent hybridisation conditions refer in general to an overnight incubation at 68° C. in a solution comprising 5×SSC (750 mM NaCl, 75 mM trisodium citrate), 50 mM sodium phosphate (pH 7.6), 5×Denhardt's solution, 10% dextran sulfate and 20 μg/ml denatured sheared salmon sperm DNA, followed by washing the filters in 0.1×SSC at about 65° C. Changes in the stringency of hybridisation are primarily accomplished through the manipulation of the SSC dilution in the washing steps (higher concentration SSC in washing buffer results in lower stringency) and the temperature (lower washing temperature results in lower stringency). For example, lower stringency conditions include washes performed at 1×SSC and at 55-60° C.

### D4. Expression Vectors

Methods which are well known to those skilled in the art may be used to construct expression vectors containing at least a fragment of any of the nucleic acids of the present invention together with appropriate transcriptional and translational control elements. These methods include in vitro recombinant DNA techniques, synthetic techniques, and in vivo genetic recombination. Such techniques are described, for example, in Sambrook et al. Molecular Cloning: A laboratory manual. Cold Spring Harbor laboratory press 1989. Correspondingly, the present invention relates also to vectors comprising a nucleic acid of the present invention, or a fragment thereof. This nucleic acid may be a member selected from a group of nucleic acids identifiable as TAG, GAG/A and/or GAG/B. Preferably, said nucleic acid is a member selected from a group represented by SEQ ID NO:s 1, 3, 5, 7, 9, 11, 13, 15, 17, 19, 21, 23, 25, 27, 29, 31 and 33, including variants, fragments or homologues thereof.

The present invention particularly contemplates recombinant expression vectors, preferably said vectors comprising a vector sequence, an appropriate prokaryotic, eukaryotic or viral or synthetic promoter sequence followed by the nucleic acid of the present invention or a fragment thereof. Preferably, the vector used for expressing the nucleic acid according to the present invention can be a vector for expression in E. coli, a yeast shuttle vector, or a yeast two-hybrid vector, a plant vector, an insect vector, a mammalian expression vector, including but not limited to, a herpes virus vector, a baculovirus vector, a lentivirus vector, a retrovirus vector, an alphavirus vector, an adenoviral vector or any combination thereof. Accordingly, in a preferred embodiment said vector is an expression vector, wherein the nucleotide sequence is operably linked to one or more control sequences allowing the expression of said sequence in prokaryotic and/or eukaryotic host cells.

In a further embodiment, the vectors of the invention are present in a host cell. The host cell is preferably a yeast, bacterial, insect, fungal, plant, fish, avian, reptilian or mammalian cell. It will be appreciated that the host cell may comprise an integrated or episomal copy of a nucleic acid according to the invention or a vector according to the invention.

In addition, the present invention provides a method for producing a polypeptide according to the invention, comprising culturing a host cell as described supra under conditions allowing the expression of the polypeptide.

It will be understood that the present invention relates also to a transgenic non-human animal comprising one or more copies of a nucleic acid of the present invention stably integrated in the genome, or an animal comprising regulatory elements that modulate the expression of a nucleic acid of the present invention.

In addition to transgenic animals, a gene may be knocked out, for instance to study effects thereof. A gene can be knocked-out by various means. Therefore, a preferred embodiment of the present invention pertains to a knock-out non-human animal comprising a deletion of one or two alleles encoding a nucleic acid according to the invention, or a animal comprising a targeted mutation in the genomic region, including regulatory sequences, comprising any of the nucleic acid sequences according to the invention. In general, a knock-out will result in the ablation of the function of the particular gene.

In an even more preferred embodiment, the present invention relates to the use of a transgenic or knock-out non-human animal according to the present invention as a model system for studying angiogenesis, and in particular proliferative diseases.

### E. ANTIBODIES

In a preferred embodiment, the invention provides an antibody specifically recognising the polypeptides of the present invention, or a specific epitope of said polypeptide. The term “epitope” refers to portions of a polypeptide having antigenic or immunogenic activity in an animal, preferably a mammal, and most preferably in a human. Epitope-bearing polypeptides of the present invention may be used to induce antibodies according to methods well known in the art including, but not limited to, in vivo immunisation, in vitro immunisation, phage display methods or ribosome display.

The antibody of the present invention relates to any polyclonal or monoclonal antibody binding to a protein of the present invention. The term “monoclonal antibody” used herein refers to an antibody composition having a homogeneous antibody population. The term is not limiting regarding the species or source of the antibody, nor is it intended to be limited by the manner in which it is made. Hence, the term “antibody” contemplates also antibodies derived from camels (Arabian and Bactrian), or the genus lama. Thus, the term “antibody” also refers to antibodies derived from phage display technology or drug screening programs. In addition, the term “antibody” also refers to humanised antibodies in which at least a portion of the framework regions of an immunoglobulin are derived from human immunoglobulin sequences and single chain antibodies as described in U.S. Pat. No. 4,946,778 and to fragments of antibodies such as F_{ab}, F_{(ab)2}, Fᵥ, and other fragments which retain the antigen binding function and specificity of the parent antibody. The term “antibody” also refers to diabodies, triabodies or multimeric (mono-, bi-, tetra- or polyvalent/mono-, bi- or polyspecific) antibodies, as well as enzybodies, i.e. artificial antibodies with enzyme activity. Combinations of antibodies with any other molecule that increases affinity or specificity, are also contemplated within the term “antibody”. Antibodies also include modified forms (e.g. mPEGylated or polysialylated form (Fernandes & Gregoriadis, 1997) as well as covalently or non-covalently polymer bound forms. In addition, the term “antibody” also pertains to antibody-mimicking compounds of any nature, such as, for example, derived from lipids, carbohydrates, nucleic acids or analogues e.g. PNA, aptamers (see Jayasena, 1999).

In specific embodiments, antibodies of the present invention cross-react with murine, goat, rat and/or rabbit homologues of human proteins and the corresponding epitopes thereof. As such, the present invention provides a method for detecting the polypeptides of the present invention, the method comprising the use of the antibodies in immunoassays for qualitatively or quantitatively measuring levels of the polypeptides of the present invention in biological samples.

In particular, the present invention relates to an antibody specifically recognising a polypeptide encoded by a nucleic acid according to the present invention, or a specific epitope of said polypeptide.

Antibodies of the present invention may act as inhibitors, agonists or antagonists of the polypeptides of the present invention.

Antibodies of the present invention may be used, for example, but not limited to, to purify, detect, target, and/or inhibit the activity of the polypeptides of the present invention, in TEC, but also PLEC, AEC or NEC, including both in vitro and in vivo diagnostic and therapeutic methods, as well as in drug screens.

### F. DIAGNOSIS

As described in the introduction, a large number of diseases including solid tumor formation are caused by a disturbance of the fine-tuned balance between signals regulating angiogenesis. The pathologies caused by disturbances in angiogenic processes include proliferative disorders including malignancies, diabetic retinopathy, rheumatoid arthritis, psoriasis, restenosis, endometriosis, impaired wound healing, and atherosclerosis. Methods which can be used for diagnosis are also further detailed in the examples section. Accordingly, the present invention relates to diagnosing a pathological condition, wherein said pathological condition is chosen from the group consisting of proliferative disorders, including tumors, diabetic retinopathy, rheumatoid arthritis, psoriasis, restenosis, endometriosis, impaired wound healing, and atherosclerosis. Correct diagnosis of a pathological condition would be beneficial for treatment of and medication to a patient suffering from said pathological condition. Furthermore, diagnosis may aid in determining a predisposition or susceptibility to a pathological condition, e.g. before onset of the pathological condition. Correspondingly, the present invention relates to a polynucleotide, polypeptide or antibody according to the invention for diagnosing a pathological condition or a susceptibility to a pathological condition. The present invention also provides the use of a polynucleotide according to the invention, such as TAG, GAG/A and/or GAG/B polynucleotides and preferably characterized by any of SEQ ID NO:s 1, 3, 5, 7, 9, 11, 13, 15, 17, 19, 21, 23, 25, 27, 29, 31 or 33, or a part thereof, for diagnosing angiogenesis, and preferably tumor endothelial cells. In a further embodiment, the present invention provides the use of an antibody specifically directed against a polypeptide according to the invention, such as TAG, GAG/A and/or GAG/B polypeptide, and preferably characterized by any of SEQ ID NO:s 2, 4, 6, 8, 10, 12, 14, 16, 18, 20, 22, 24, 26, 28, 30, 32 or 34, or a part thereof, for diagnosing a pathological condition such as a proliferative disorders and/or impaired angiogenesis.

In a preferred embodiment, the present invention relates to a method of diagnosing a pathological condition or a susceptibility to a pathological condition in a subject comprising the steps of:
(a) determining the over- or under-expression of a polynucleotide or a polypeptide according to the invention in a biological sample relative to the expression in a control sample, and,
(b) diagnosing a pathological condition or a susceptibility to a pathological condition based on the over- or under-expression of said polynucleotide or said polypeptide in said biological sample relative to the expression in a control sample.

The term “biological sample” refers to a sample that is tested for the presence, abundance, quality or an activity of a molecule of interest, such as a polypeptide according to the invention, a polynucleotide encoding a polypeptide according to the invention, or an agent or compound that modifies or modulates the activity of a polypeptide according to the invention. A sample containing a molecule of interest, may be obtained in numerous ways known in the art. Virtually any sample may be analysed using the method according to the present specification including cell lysates, purified genomic DNA, body fluids such as from a human or animal, clinical samples, etc. Thus, a “biological sample” contemplates a sample obtained from an organism or from components (e.g., cells) of an organism. The sample may be of any biological tissue or fluid. Usually, the sample is a biological or a biochemical sample. Frequently the sample will be a “clinical sample” which is a sample derived from a patient. Such samples include, but are not limited to, sputum, cerebrospinal fluid, blood, blood fractions such as serum including foetal serum (e.g., SFC) and plasma, blood cells (e.g., white cells), tissue or fine needle biopsy samples, urine, peritoneal fluid, and pleural fluid, or cells there from. Biological samples may also include sections of tissues such as frozen sections taken for histological purposes. The sample can be, for example, also a physiological sample. The term “tissue” as used herein refers to cellular material from a particular physiological region. The cells in a particular tissue can comprise several different cell types. A non-limiting example of this would be tumor tissue that comprises capillary endothelial cells and blood cells, all contained in a given tissue section or sample. It will be appreciated from the invention that in addition to solid tissues, the term “tissue” is also intended to encompass non-solid tissues, such as blood.

A “control sample” or “standard” relates to a sample of which the expression level, amount and/or abundance of a polynucleotide, nucleic acid, polypeptide and/or activity of a polypeptide is known, or has been determined previously. As such, the control sample may be derived from a “healthy” person, i.e. a person diagnosed previously as not suffering or predisposed from the pathological condition(s) at issue. Alternatively, the control sample may be derived from a “diseased” person, i.e. a person diagnosed previously as suffering or predisposed from the pathological condition(s) at issue. The sample may be spiked with a known amount of molecules. In a further alternative, the control sample may be synthetic, i.e. not derived from a person, but comprising a known amount of molecules.

In a preferred embodiment, the present invention relates to a method of diagnosing a pathological condition or a susceptibility to a pathological condition, said method comprising:
(a) contacting a biological sample with a probe specific for any of the nucleic acids according to the invention, such as TAG, GAG/A and/or GAG/B nucleic acids, and preferably SEQ ID NO:s 1, 3, 5, 7, 9, 11, 13, 15, 17, 19, 21, 23, 25, 27, 29, 31 or 33, or a part thereof;
(b) detecting binding of said probe to said nucleic acids according to the invention, such as TAG, GAG/A and/or GAG/B nucleic acids, and preferably SEQ ID NO:s 1, 3, 5, 7, 9, 11, 13, 15, 17, 19, 21, 23, 25, 27, 29, 31 or 33, or a part thereof present in said biological sample;
(c) comparing the binding detected in step (b) with a standard,
   wherein a difference in binding relative to the standard is diagnostic of a pathological condition or a susceptibility to a pathological condition.

In another embodiment, the present invention relates to a method for targeting a diagnostic agent to tumor-associated vasculature in an animal, preferably a human, having a vascularized tumor, comprising: administering a diagnostic agent to the animal, wherein the diagnostic agent comprises an operatively attached targeting compound, and wherein the targeting compound recognizes and binds to a TAG, said TAG preferably being chosen from the group characterized by any of SEQ ID NO:s 1 to 34.

As used herein, the “diagnostic agent” relates to an agent comprising two functional moieties, i.e. a first moiety enabling detection (detection compound) and a second moiety enabling binding to the molecule to be diagnosed (targeting compound). In a preferred embodiment, the present invention relates to a method as described herein, wherein said targeting compound is an antibody and the detection compound is a paramagnetic, radioactive or fluorogenic molecule that is detectable upon imaging.

In another embodiment, the present invention relates to a method of identifying regions of (neo)angiogenesis in an animal, preferably a human, comprising:
administering to an animal a diagnostic agent comprising an antibody variable region which specifically binds to a polypeptide according to the invention, such as TAG, GAG/A and/or GAG/B polypeptide, or a part thereof, said polypeptide preferably selected from the group consisting of SEQ ID NO:s 2, 4, 6, 8, 10, 12, 14, 16, 18, 20, 22, 24, 26, 28, 30, 32 and 34, including parts thereof;
detecting the diagnostic agent in the patient; and
   thereby identifying regions of (neo)angiogenesis in the patient.

In a further embodiment, the present invention relates to a method of screening for (neo)angiogenesis in a patient, comprising:
(a) contacting a biological sample with a molecule comprising an antibody variable region which specifically binds to a polypeptide according to the invention, such as TAG, GAG/A and/or GAG/B polypeptide, or a part thereof, said polypeptide preferably selected from the group consisting of SEQ ID NO:s 2, 4, 6, 8, 10, 12, 14, 16, 18, 20, 22, 24, 26, 28, 30, 32 and 34, including parts thereof; and
(b) detecting material in the biological sample that is cross-reactive with the molecule, and
   wherein detection of cross-reactive material indicates neo-angiogenesis in the patient.

The invention also provides a method of screening for neo-angiogenesis in a patient, comprising:
(a) detecting an expression product of at least one gene according to the invention, such as TAG, GAG/A and/or GAG/B genes, in a first tissue sample of a patient, wherein said at least one gene is preferably selected from the group consisting of SEQ ID NO:s 1, 3, 5, 7, 9, 11, 13, 15, 17, 19, 21, 23, 25, 27, 29, 31 and 33, including parts thereof; and
(b) comparing expression of the expression product of said at least one gene in the first tissue sample with expression of the expression product of the at least one gene in a second tissue sample which is normal,
   wherein an increased expression of the expression product of the at least one gene in the first tissue sample relative to the second tissue sample identifies the first tissue sample as likely to be neo-angiogenic.

### F.1 Diagnosing Nucleic Acids

Also, the present invention relates to a method for diagnosing a pathological condition or a susceptibility to a pathological condition, comprising the steps of:
(a) detecting an expression product of at least one gene according to the invention in a first biological sample suspected of a pathological condition, wherein said at least one gene characterized by a polynucleotide according to the invention, such as TAG, GAG/A and/or GAG/B polynucleotides, and preferably selected from the group consisting of SEQ ID NO:s 1, 3, 5, 7, 9, 11, 13, 15, 17, 19, 21, 23, 25, 27, 29, 31 and 33, including parts thereof; and
(b) comparing expression of the expression product of at least one gene in the first biological sample with expression of the expression product of the at least one gene in a second biological sample which is normal,
   wherein a difference in expression of the expression product of the at least one gene in the first biological sample relative to the second biological sample identifies the first biological sample as likely to be pathological or susceptible to a pathological condition.

In a preferred embodiment, the present invention relates to a method for diagnosing a biological sample as likely to be neoplastic or vascularized tumors, comprising the steps of:
(a) detecting an expression product of at least one gene in a first biological sample suspected of being neoplastic wherein said expression product of at least one gene is characterized by a polynucleotide according to the invention, such as TAG, GAG/A and/or GAG/B polynucleotides, and preferably selected from the group consisting of SEQ ID NO:s 1, 3, 5, 7, 9, 11, 13, 15, 17, 19, 21, 23, 25, 27, 29, 31 and 33, including parts thereof; and
(b) comparing expression of the at least one gene in the first biological sample with expression of the at least one gene in a second biological sample which is normal,
   wherein increased expression of the at least one gene in the first biological sample relative to the second biological sample identifies the first biological sample as likely to be neoplastic.

In another preferred embodiment, the present invention relates to a method for diagnosing impaired wound healing, comprising the steps of:
(a) detecting an expression product of at least one gene in a first biological sample suspected of having impaired wound healing, wherein said at least one gene is characterized by a polynucleotide according to the invention, such as TAG, GAG/A and/or GAG/B polynucleotides, and preferably selected from the group consisting of GAG/A or GAG/B, SEQ ID NO:s 1, 3, 5, 7, 9, 11, 13, 15, 17, 19, 21, 23, 25, 27, 29, 31 and 33, or a part thereof; and
(b) comparing expression of the at least one gene in the first biological sample with expression of the at least one gene in a second biological sample which is normal,
   wherein differential, e.g. decreased or increased, expression of the at least one gene in the first biological sample relative to the second biological sample identifies the first biological sample as likely to be impaired in wound healing.

Difference in expression levels of genes can be determined by any method known in the art, such as for instance quantitative PCR or hybridisation techniques. The difference in expression qualifying a first biological sample as likely to be pathogenic, e.g. neoplastic or impaired in wound healing is at least 2-fold, relative to the expression level in a second biological sample which is normal. Accordingly, the present invention relates to a method as described herein, wherein the difference in expression, the increased expression or the decreased expression of the at least one gene in the first biological sample relative to the second biological sample is at least 2-fold, and preferably 5-fold or even more, such as 10-fold. Preferably, the expression product for which the expression level is determined, is RNA, e.g. mRNA, preferably encoding for SEQ ID NO 2, 4, 6, 8, 10, 12, 14, 16, 18, 20, 22, 24, 26, 28, 30, 32 or 34, or GAG/A or GAG/B or a part thereof.

In a further preferred embodiment, the present invention relates to the use of a nucleic acid characterized by any of SEQ ID NO 11, or a part thereof, for diagnosing angiogenesis, and preferably tumor endothelial cells.

### F.2 Diagnosing Polypeptides

In a preferred embodiment, the present invention relates to a method of diagnosing a pathological condition or a susceptibility to a pathological condition, said method comprising:
(a) contacting a biological sample with an antibody specific for a polypeptide according to the invention, such as TAG, GAG/A and/or GAG/B polypeptides, and preferably chosen from the group consisting of SEQ ID NO:s 2, 4, 6, 8, 10, 12, 14, 16, 18, 20, 22, 24, 26, 28, 30, 32 and 34, including parts thereof;
(b) detecting binding of said antibody to said polypeptide, or a part thereof, present in said biological sample;
(c) comparing the binding detected in step (b) with a standard,
   wherein a difference in binding relative to the standard is diagnostic of a pathological condition or a susceptibility to a pathological condition. The method of diagnosing a pathological condition according to the invention may comprise FACS analysis, e.g. the detection step is performed by using FACS, or the use of protein or antibody arrays, ELISA, or immunoblotting.

The present invention also relates to a method for diagnosing a pathological condition or a susceptibility to a pathological condition, comprising the steps of:
(a) detecting an expression product of at least one gene in a first tissue sample suspected of pathological, wherein said expression product of at least one gene is selected from the genes according to the invention, such as TAG, GAG/A and/or GAG/B genes, and preferably selected from the group consisting of SEQ ID NO:s 2, 4, 6, 8, 10, 12, 14, 16, 18, 20, 22, 24, 26, 28, 30, 32 and 34, including parts thereof; and
(b) comparing expression of the expression product of at least one gene in the first tissue sample with expression of the expression product of the at least one gene in a second tissue sample which is normal,
   wherein a difference in expression of the expression product of the at least one gene in the first tissue sample relative to the second tissue sample identifies the first tissue sample as likely to be pathological or susceptible to a pathology.

In a further embodiment, the present invention relates to a method for diagnosing vascularized tumors, comprising the steps of:
(a) detecting an expression product of at least one gene in a first biological sample suspected of being neoplastic, wherein said expression product of at least one gene is characterized by a polypeptide according to the invention, such as TAG, GAG/A and/or GAG/B polypeptides, and preferably chosen from the group consisting of SEQ ID NO:s 2, 4, 6, 8, 10, 12, 14, 16, 18, 20, 22, 24, 26, 28, 30, 32 and 34, including a part thereof; and
(b) comparing expression of the at least one gene in the first biological sample with expression of the at least one gene in a second biological sample which is normal,
   wherein increased expression of the at least one gene in the first biological sample relative to the second biological sample identifies the first biological sample as likely to be neoplastic.

In another embodiment, the present invention relates to a method for diagnosing impaired wound healing, comprising the steps of:
(a) detecting an expression product of at least one gene in a first biological sample suspected of having impaired wound healing, wherein said expression product of at least one gene is characterized by a polynucleotide according to the invention, such as TAG, GAG/A and/or GAG/B polynucleotides, and preferably selected from the group consisting of SEQ ID NO:s 2, 4, 6, 8, 10, 12, 14, 16, 18, 20, 22, 24, 26, 28, 30, 32 and 34, including parts thereof; and
(b) comparing expression of the at least one gene in the first biological sample with expression of the at least one gene in a second biological sample which is normal,
   wherein decreased expression of the at least one gene in the first biological sample relative to the second biological sample identifies the first biological sample as likely to be impaired in wound healing.

It will be appreciated that the first and second biological samples are preferably derived from human. Furthermore, the first and second biological samples may be derived from the same human, e.g. the first biological sample is derived from a tissue suspected of being neoplastic, while the second biological sample is derived from another, non-malignant tissue.

In the diagnostic methods of the invention, the step of detecting may be performed by any diagnostic technique, known by the person skilled in the art, and preferably using immunoassays, which may include the use of antibodies, such as Western blot, ELISA, RIA, immuno(histo)chemical assay, and/or hybridisation assays such as Southern/Northern/Virtual Northern blotting techniques and/or oligonucleotide arrays and microarrays, and/or specific amplification techniques, such as PCR, NASBA or TMA technologies, and any combination of the above.

In another preferred embodiment, the present invention relates to the use of an antibody specifically directed against a protein characterized by SEQ ID NO: 12, or a part thereof, for diagnosing proliferative disorders and/or angiogenesis.

### F.3 Detecting Endothelial Cells

The molecules identified in the present invention may support the detection of endothelial cells. Accordingly, the present invention also relates to a method for identifying endothelial cells, comprising:
(a) contacting a population of cells with at least one molecule comprising a variable region which binds specifically to a polypeptide according to the invention, such as TAG, GAG/A and/or GAG/B polypeptides, and preferably selected from the group consisting of SEQ ID NO:s 2, 4, 6, 8, 10, 12, 14, 16, 18, 20, 22, 24, 26, 28, 30, 32 and 34, or to any other polypeptide identified in the present invention as endothelial cell specific, or a part thereof;
(b) detecting cells in the population which have bound to said molecules; and
(c) identifying cells which are bound to said one or more molecules as endothelial cells.

Also, the present invention relates to a method for identifying endothelial cells, comprising:
(a) contacting cDNA or mRNA of a population of cells with one or more nucleic acid hybridization probes which are complementary to a cDNA or mRNA for a gene characterized by a polynucleotide according to the invention, such as TAG, GAG/A and/or GAG/B polynucleotides, and preferably selected from the group consisting of SEQ ID NOs 1, 3, 5, 7, 9, 11, 13, 15, 17, 19, 21, 23, 25, 27, 29, 31 and 33, or GAG/A or GAG/B, including parts thereof,
(b) detecting cDNA or mRNA which have specifically hybridized to said nucleic acid hybridization probes; and
   identifying cells whose nucleic acids specifically hybridized to said nucleic acid hybridization probes as endothelial cells.

### F.4 Selection of Endothelial Cells

The staining or selection of endothelial cells may be accomplished by staining with anti-CD31 and anti-CD34 antibodies; and isolated by positive selection e.g., by using goat anti-mouse IgG coated paramagnetic beads. Hence, in one embodiment, the present invention also provides for the selection of endothelial cells from human tissues for the purpose of gene expression by using the combination of anti-CD31 and anti-CD34 antibodies.

### G. TREATMENT AND MEDICAMENTS

The terms “treatment”, “treating”, and the like, as used herein include amelioration or elimination of a developed disease or condition once it has been established or alleviation of the characteristic symptoms of such disease or condition. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of the compound or composition of the invention to a patient that is not at the time of administration afflicted with the disease or condition. “Preventing” also encompasses preventing the recurrence or relapse-prevention of a disease or condition or of symptoms associated therewith, for instance after a period of improvement.

As used herein, the term “medicament” also encompasses the terms “drug”, “therapeutic”, “potion” or other terms which are used in the field of medicine to indicate a preparation with a therapeutic or prophylactic effect.

To prepare the pharmaceutical compositions, comprising the compounds, described herein, such as nucleic acids, polypeptides, antisense oligonucleotides, siRNA, antibodies and the like, an effective amount of the active ingredients, in acid or base addition salt form or base form, may be combined in admixture with a pharmaceutically acceptable carrier, which can take a wide variety of forms depending on the form of preparation desired for administration. These pharmaceutical compositions are desirably in unitary dosage form suitable, for administration orally, nasal, rectally, percutaneously, transdermally, by parenteral, intramuscular, intravascular injection or intrathecal administration. The pharmaceutical compounds for treatment are intended for parenteral, topical, oral or local administration and generally comprise a pharmaceutically acceptable carrier and an amount of the active ingredient sufficient to reverse or prevent the adverse effects of pathological conditions connected with impaired angiogenesis or proliferative diseases. The carrier may be any of those conventionally used and is limited only by chemico-physical considerations, such as solubility and lack of reactivity with the compound, and by the route of administration.

Hence, the present invention relates to the use of a nucleic acid, polypeptide, antibody, siRNA, or antisense oligonucleotide according to the invention for the preparation of a medicament for treating a pathological condition, e.g. preventing, treating and/or alleviating proliferative disorders, or for stimulating angiogenesis. In addition, the present invention relates to a method for the production of a composition comprising the steps of admixing a nucleic acid, polypeptide, antibody, siRNA, or antisense oligonucleotide according to the invention with a pharmaceutically acceptable carrier. The present invention relates specifically to the use of an inhibitor of HMGB1 for the preparation of a medicament for preventing, treating and/or alleviating proliferative disorders. In particular, the present invention relates to the use as described above, wherein said inhibitor is an anti-HMGB1antibody. In an alternative embodiment, the present invention relates to the use as described herein, wherein said inhibitor is siRNA duplex, said siRNA duplex complexes with a nucleic acid comprising a nucleotide sequence which is at least 90% identical to SEQ ID NO: 11 or a part thereof.

### G.1 Treating Proliferative Diseases

### Antibodies

The present invention demonstrated that the selected TAG markers are related to the process of angiogenesis. In vitro as well as in vivo bioassays proved that therapeutic agents directed against the TAG markers showed inhibitory effects. In particular, antibodies inhibited endothelial tube formation in an in vitro collagen-gel-based sprout-formation assay. Also, antibodies directed against the polypeptides of the invention specifically inhibited the developing chorioallantoic membrane (CAM) of the chick embryo. Furthermore, antibodies inhibited tumor growth in a mouse model.

Accordingly, the present invention relates to an antibody specifically recognizing a polypeptide of the invention for use as a medicament. The present invention also contemplates a method as described herein, wherein the therapeutic agent is an antibody directed specifically against any of the polypeptides according to the invention, such as TAG, GAG/A and/or GAG/B polypeptides, and preferably selected from the group consisting of SEQ ID NO:s 2, 4, 6, 8, 10, 12, 14, 16, 18, 20, 22, 24, 26, 28, 30, 32 and 34, including parts thereof.

As used in the present invention, a “therapeutic agent” is a compound which is able to interfere with the expression, whether up or down, of a gene according to the invention. The term “therapeutic agent” also contemplates a compound which is able to interfere with the activity an expression product from a gene according to the invention. The therapeutic agent according to the invention may comprise an anticellular moiety capable of killing or suppressing the growth or cell division of targeted endothelial cells. The anti-cellular agent moiety may be chosen from the group consisting of a chemotherapeutic agent, a radioisotope, a cytotoxin, a steroid, an antimetabolite, an anthracycline, a vinca alkaloid, an antibiotic, an alkylating agent, or an epipodo-phyllotoxin, or a plant-, fungus- or bacteria-derived toxin. The therapeutic agent may be antibodies directed against the polypeptides according to the invention, or parts thereof, and said antibodies are coupled to anti-cellular agents. A therapeutic agent is intended to treat or alleviate a pathological condition, such as proliferative diseases or disorders, including cancer, arthritis, diabetes, psoriasis and endometriosis or ischemia, heart failure, infertility, ulcer formation and impaired wound healing.

The term “expression” according to the present invention comprises the activity of gene and its gene product, including transcription into mRNA and/or translation of the mRNA into protein. It will be appreciated that an “expression product” of a gene encompasses the mRNA but also the protein derived therefrom, as well as the activity, function and mode of action of said protein.

In a preferred embodiment, the present invention relates to a method for inhibiting a pathological condition, such as proliferative diseases or disorders in a subject comprising such a pathological condition, e.g. a proliferative disease or disorder, comprising: administering to the subject an effective amount of a composition comprising an antibody which specifically binds to an epitope of any of the polypeptides according to invention, such as TAG, GAG/A and/or GAG/B polypeptides, and preferably comprising or consisting of SEQ ID NO:s 2, 4, 6, 8, 10, 12, 14, 16, 18, 20, 22, 24, 26, 28, 30, 32 or 34, or a part thereof.

In a further preferred embodiment, the present invention relates to a method for inducing an immune response to a polypeptide according to the invention in a mammal, such as TAG, GAG/A and/or GAG/B polypeptides, comprising: administering to a subject who has or is at risk of developing a proliferative disease or disorder a protein according to the invention, or a nucleic acid encoding a protein according to the invention, wherein said protein is preferably selected from the group consisting of TAG, GAG/A and/or GAG/B polypeptides, and preferably comprising or consisting of SEQ ID NO:s 2, 4, 6, 8, 10, 12, 14, 16, 18, 20, 22, 24, 26, 28, 30, 32 and 34, including parts thereof; whereby a humoral or cellular immune response to the protein according to the invention is raised in the human subject. The therapeutic methods according to the invention may further comprise administering to the subject an immune adjuvant to augment the immune response.

Preferably, the present invention relates to therapeutic methods according to the invention, wherein the proliferative disorders are vascularized tumors possibly comprising enhanced angiogenesis and/or tumor endothelial cells. As such, the present invention relates to a method of treating a vascularized tumor, comprising the step of: contacting cells of the vascularized tumor with an antibody, wherein the antibody specifically binds to an extracellular epitope of a polypeptide according to invention, such as TAG, GAG/A and/or GAG/B polypeptides, and said polypeptide preferably comprising or consisting of any of SEQ ID NO:s 2, 4, 6, 8, 10, 12, 14, 16, 18, 20, 22, 24, 26, 28, 30, 32 or 34, or a part thereof; whereby immune destruction of cells of the vascularized tumor is triggered.

Also, the present invention relates to a method for targeting a therapeutic agent to tumor-associated vasculature in an animal having a vascularized tumor, comprising: administering a therapeutic agent to the animal, wherein the therapeutic agent compound comprises a targeting compound, and wherein the targeting compound recognizes and binds to a polypeptide or polynucleotide according to the invention, preferably a TAG according to the invention, such as a TAG being chosen from the group characterized by any of SEQ ID NO:s 1-34. Preferably, the targeting compound is an antibody. Said antibody may recognize and bind to a TAG which is present on the surface of the tumor-associated endothelial cell, preferably at a higher concentration than on the surface of normal, non-tumor associated endothelial cells.

Antisense—Ribozyme—siRNA Technology

In a further preferred embodiment, the present invention relates to a method for treating or alleviating proliferative diseases or disorders, comprising the use of a therapeutic agent which allows interfering with the expression of a nucleic acid or a polypeptide according to the invention, in a patient.

Antisense technology can be used to control gene expression, for example for inhibition of gene expression, i.e. transcription, as described in the art. As such, antisense nucleic acids can be used as antagonist compounds, and may be employed to regulate the effects of the polypeptides of the present invention on the modulation of angiogenesis, and in particular the onset of angiogenesis in malignancies, both in vitro and in vivo.

Thus, in a further embodiment, the present invention provides an antisense nucleic acid directed against the nucleic acid according to the present invention, or a part thereof. Such antisense nucleic acids can be constructed by recombinant DNA technology methods standard in the art. In a preferred embodiment, the present invention provides a vector comprising a polynucleotide sequence as described herein encoding an antisense nucleic acid. In a more preferred embodiment, said vector is an expression vector wherein the antisense polynucleotide sequence is operably linked to one or more control sequences allowing the expression, i.e. transcription, of said sequence in prokaryotic and/or eukaryotic host cells.

Potential antagonists according to the invention also include catalytic RNA, or a ribozyme. Ribozymes cleave mRNA at site-specific recognition sequences and can be used to destroy mRNAs corresponding to the nucleic acids of the present invention. The construction and production of ribozymes is well known in the art. As in the antisense approach, ribozymes of the invention can be used as antagonist compounds, and can be delivered to cells to, for example, inhibit in vitro or in vivo angiogenesis or stimulate the induction of endothelial activation effects of the polypeptides of the present invention. Similarly, the nucleic acids of the present invention, the RNA molecules derived thereof, functional equivalent parts or fragments thereof can contain enzymatic activity or can squelch RNA binding polypeptides or can exert effects as antisense agents by binding the endogenous sense strand of mRNA, all of which can modulate angiogenesis, preferably the down regulation of TEC specific genes.

The invention further provides the nucleic acid sequences for controlling gene expression using RNA interference (i.e. siRNA, formerly known as double stranded RNA or dsRNA). It has been described in the art (WO 99/32169) that providing siRNA to a target cell can result in the down regulation of the translation/expression of any desired RNA sequence that may be present in said cell. As such, the nucleic acids of the present invention can be used as antagonistic or agonistic compounds, and may be employed to regulate the effects of the polypeptides of the present invention on the modulation of angiogenesis and in particular the down regulation of TEC over-expressed genes (i.e., overexpressed in TEC relative to NEC and PLEC), both in vitro and in vivo. Moreover, the present invention relates to siRNA for use as a medicament, characterised that said siRNA agonises or antagonises angiogenesis by said polynucleotide sequences. Accordingly, the present invention relates to a cell, in which the polynucleotide sequences comprising the nucleic acids sequences as described herein have been introduced.

The present invention also contemplates a method as described herein, wherein the therapeutic agent is an antisense molecule, a ribozyme or an siRNA directed specifically against a polynucleotide according to the invention, such as TAG, GAG/A and/or GAG/B polynucleotides, and preferably any of SEQ ID NO:s 1, 3, 5, 7, 9, 11, 13, 15, 17, 19, 21, 23, 25, 27, 29, 31 or 33, or a part thereof.

G.2 Method for Treating Disorders Connected with Insufficient Angiogenesis

The methods of the invention identified genes and gene products involved in disproportionate angiogenesis, but also clarified the role of various genes and gene products in normal physiological angiogenic processes, e.g. active angiogenesis in wound healing. This in turn elucidated the role of these genes and gene products in cases of insufficient angiogenesis. Therefore, the present invention also relates to the therapeutic agents to stimulate angiogenesis e.g. vascular proliferation. This may be beneficial to patients having wounds, impaired wound healing, ischemia, heart failure, infertility, or ulcer formation. Accordingly, the present invention encompasses nucleic acids or polypeptides according to the invention for use as a medicament. The present invention further encompasses a method for treating or alleviating a pathological condition resulting or connected with insufficient angiogenesis, such as impaired wound healing, ischemia, heart failure, infertility, ulcer formation, comprising the use of a therapeutic agent which allows to interfere with, preferably increase the expression of a nucleic acid or a polypeptide according to the invention, in a patient. Hence, the present invention relates to a method for stimulating vascular proliferation comprising: administering to a subject with a insufficient angiogenesis a protein according to the invention, such as TAG, GAG/A and/or GAG/B protein, or a polynucleotide or nucleic acid encoding a protein according to the invention, or a functional fragment thereof, wherein said protein according to the invention is characterized by a TAG, GAG/A and/or GAG/B polypeptide according to the invention, and preferably chosen from the group consisting of SEQ ID NO:s 2, 4, 6, 8, 10, 12, 14, 16, 18, 20, 22, 24, 26, 28, 30, 32 and 34, including parts thereof; whereby vascular proliferation is promoted.

The present invention also contemplates the use of the polynucleotides or polypeptides of the present inventions in persons having wounds or scar tissue in order to stimulate vascular proliferation. As such, the present invention relates to a method for stimulating vascular proliferation comprising: administering to a subject with a wound or scar tissue a protein according to the invention or nucleic acid encoding a protein according to the invention, wherein the protein according to the invention is preferably characterized by a TAG, GAG/A and/or GAG/B protein, and preferably chosen from the group consisting of SEQ ID NO:s 2, 4, 6, 8, 10, 12, 14, 16, 18, 20, 22, 24, 26, 28, 30, 32 and 34, including parts thereof; whereby wound healing and the break down of scar tissue is promoted.

In another embodiment, the present invention contemplates a method for regulating or modulating angiogenesis, and in particular inducing angiogenesis comprising:
(a) introducing a nucleic acid or an expression vector comprising a nucleic acid according to the present invention in a desired target cell, in vitro or in vivo,
(b) expressing said nucleic acid, and,
(c) regulating angiogenesis by the products expressed by said nucleic acid or the product of said expression vector.

In a preferred embodiment, the invention provides polypeptides, including protein fusions, or fragments thereof, for regulating angiogenesis, and in particular induction of endothelial cell activity, in vitro or in vivo. For example, the induction of endothelial cell activity may occur as a direct result of administering polypeptides to mammalian, preferably human, cells. Delivering compositions containing the polypeptide of the invention to target cells, may occur via association via heterologous polypeptides, heterologous nucleic acids, toxins, or pro-drugs via hydrophobic, hydrophilic, ionic and/or covalent interactions.

In another preferred embodiment the present invention provides a gene therapy method for treating, alleviating or preventing disorders and diseases involving pathological disturbance of angiogenesis. The gene therapy methods relate to the introduction of nucleic acid sequences into an animal to achieve expression of a polypeptide of the present invention. This method requires a nucleic acid, which codes for a polypeptide of the invention that is operatively linked to a promoter or any other genetic element necessary for the expression of the polypeptide in the target tissue. Such gene therapy and delivery techniques are known in the art, see, for example, EP-A-0 707 071.

In a further embodiment, the nucleic acid of the invention is delivered as a naked polynucleotide. The term naked nucleic acid refers to sequences that are free from any delivery vehicle that acts to assist, promote or facilitate entry into a cell, including viral sequences, viral particles, liposome formulations, lipofectin or precipitating agents and the like. The naked nucleic acids can be delivered by any method known in the art, including, but not limited to, direct needle injection at the delivery site, intravenous injection, topical administration, catheter infusion, and so-called “gene guns”.

In another embodiment, the nucleic acids of the present invention may be delivered with delivery vehicles such as viral sequences, viral particles, liposome formulations, lipofectin or precipitating agents and the like. Viral vectors that can be used for gene therapy applications include, but are not limited to, a herpes virus vector, a baculovirus vector, a lentivirus vector, a retrovirus vector, an alphavirus vector, an adeno-associated virus vector or an adenoviral vector or any combination thereof.

Delivery of the nucleic acids into a subject may be either direct, in which case the subject is directly exposed to the nucleic acid or nucleic acid-carrying vectors, or indirect, in which case cells are first transformed with the nucleic acids in vitro, and then transplanted into the subject. These two approaches are known, respectively, as in vivo or ex vivo gene therapy and are well described. In addition, the polypeptides according to the invention can be used to produce a biopharmaceutical. The term “biopharmaceutical” relates to a recombinantly or synthetically produced polypeptide or protein. Means to recombinantly or synthetically produce polypeptides or proteins are well known in art, such as for example described in Sambrook et al. (1989). Said biopharmaceutical can be applied in vivo, such as for example intravenously or subcutaneously. Alternatively, said biopharmaceutical can be applied in vivo, such as for example by isolating cells of patient, after which the cells are treated with said biopharmaceutical. Subsequently, said treated cells are re-introduced into said patient.

In a more preferred embodiment, the present invention provides a gene therapy method for stimulating vascular proliferation comprising the use of vectors as described herein.

Cells into which nucleic acids or polypeptides of the present invention can be introduced, for example for therapeutic purposes, encompass any desired available cell type, including but not limited to endothelial cells, progenitors of endothelial cells, and various stem cells, in particular endothelial stem cells.

In a preferred embodiment, the invention provides a method for treating, alleviating or preventing disorders involving pathological disturbance of angiogenesis comprising the use of a molecule, which allows interfering with the expression of a polynucleotide and/or expression and/or functional activity of a polypeptide of the present invention in a patient in need of such a treatment. The invention also provides a method for regulating cell proliferation, said method comprising introduction of a nucleic acid or an expression vector according to the invention in a desired target cell.

Accordingly, the present invention relates to a cell, in which the polynucleotide sequences comprising the nucleic acids sequences as described herein have been introduced. It will be understood that said cell could be used as a medicament, in that said cell could be introduced in a patient suffering from pathologies related to the disturbance of angiogenesis. Repopulating with said cells will be beneficial to the patient.

Hence, the present invention relates to the use of a polynucleotide encoding a polypeptide comprising an amino acid sequence which is at least 65% identical to any of the polypeptides according to the invention, such as TAG, GAG/A and/or GAG/B polypeptides, and preferably any of SEQ ID NO:s 2, 4, 6, 8, 10, 12, 14, 16, 18, 20, 22, 24, 26, 28, 30, 32 or 34, for stimulating angiogenesis.

In addition, the present invention provides the use of a polypeptide comprising an amino acid sequence which is at least 65% identical to any of the polypeptides according to the invention, such as TAG, GAG/A and/or GAG/B polypeptides, and preferably any of SEQ ID NO:s 2, 4, 6, 8, 10, 12, 14, 16, 18, 20, 22, 24, 26, 28, 30, 32 or 34, for stimulating angiogenesis.

### H. METHODS FOR IDENTIFYING MODULATORS OF ANGIOGENESIS

The present invention characterized for the first time various molecules that are involved in angiogenesis, including normal angiogenic processes, e.g. GAG/A molecules, as well as pathological angiogenesis, e.g. TAG molecules. It was furthermore shown in the present invention that inhibition of these TAG molecules inhibited or impaired angiogenesis. Hence, the present invention enables the further identification of therapeutic agents able to modulate angiogenesis.

As used herein, a “modulator” and “agent that modulates”, which are used interchangeably herein, refer to any compound that “modulates”, i.e. modulate, change, or interfere with angiogenesis, including excessive angiogenesis as well as insufficient angiogenesis, such as an agent that increases or decreases the expression of a gene of the invention, increases or decreases the activity of a gene product of the invention, or any compound that increases or decreases the intracellular response initiated by an active form of the gene product of the invention, or any compound that increases or decreases angiogenesis. A modulator includes an agonist, antagonist, inhibitor or inverse agonist of angiogenesis. The modulator according to the invention may aid in preventing, treating or alleviating a pathological condition. A modulator can be a protein, a nucleic acid, an antibody or fragment thereof, such as an antigen-binding fragment, a protein, a polypeptide, a peptide, a lipid, a carbohydrate, a small inorganic or organic molecule, etc. Candidate modulators can be natural or synthetic compounds, including, for example, small molecules, compounds contained in extracts of animal, plant, bacterial or fungal cells, as well as conditioned medium from such cells. In this respect, it will be understood that either the nucleic acid itself or the product encoded by said nucleic acid, e.g. the mRNA or the polypeptide, can interfere with the mechanisms involved in angiogenesis. Methods to be used in screening for modulators are further detailed in the examples section. Preferably, the candidate modulator inhibits the expression or activity of any of said genes or proteins according to the invention, such as TAG, GAG/A and/or GAG/B genes or proteins, and preferably characterized by SEQ ID NO:s 1 to 34. The modulators of angiogenesis may be used as drugs to treat pathological conditions linked with perturbed angiogenesis, e.g. impaired or excessive angiogenesis.

Accordingly, the present invention provides a method of identifying an agent that modulates a pathological condition, such as proliferative diseases or disorders, said method comprising:
(a) contacting a cell line expressing, and preferably over-expressing, a polynucleotide comprising any of the polynucleotides according to the invention, such as TAG, GAG/A and/or GAG/B polynucleotides, and preferably any of the polynucleotides characterized by SEQ ID NO:s 1, 3, 5, 7, 9, 11, 13, 15, 17, 19, 21, 23, 25, 27, 29, 31 or 33 in the presence and absence of a candidate modulator under conditions permitting the interaction of said candidate modulator with said cell; and,
(b) measuring the expression of said polynucleotide,
   wherein a modulation in expression of said polynucleotide, in the presence of said candidate modulator, relative to the expression in the absence of said candidate modulator identifies said candidate modulator as an agent that modulates a pathological condition such as proliferative diseases or disorders.

In a further embodiment, the present invention provides a method of identifying an agent that modulates a pathological condition, such as proliferative diseases or disorders, said method comprising:
(a) contacting a cell line expressing, and preferably over-expressing any of the polypeptides according to the invention, such as TAG, GAG/A and/or GAG/B polypeptides, and preferably any of the polypeptides comprising the amino acid sequence characterized by any of SEQ ID NO:s 2, 4, 6, 8, 10, 12, 14, 16, 18, 20, 22, 24, 26, 28, 30, 32 or 34 in the presence and absence of a candidate modulator under conditions permitting the interaction of said candidate modulator with said cell; and
(b) measuring the expression of said polypeptide,
   wherein a modulation in expression of said polypeptide, in the presence of said candidate modulator, relative to the expression in the absence of said candidate modulator identifies said candidate modulator as an agent that modulates pathological condition, such as proliferative diseases or disorders.

In an even further embodiment, the present invention provides a method for screening agents for preventing, treating or alleviating pathological condition, such as proliferative diseases or disorders comprising the steps of:
(a) contacting the agent to be screened with a polynucleotide or a polypeptide according to the invention, and,
(b) determining whether said agent affects the expression activity of said polynucleotide or said polypeptide.

In a preferred embodiment, the present invention provides a method for screening agents that interact with the polypeptide according to the invention, or a variant or a derivative thereof, or an immunologically active and/or functional fragment thereof, comprising:
(a) combining the polypeptide according to the invention, or a variant or a derivative thereof, or an immunologically active and/or functional fragment thereof, with an agent, to form a complex, and,
(b) detecting the formation of a complex,
   wherein the ability of the agent to interact with said polypeptide, or a variant or a derivative thereof, or an immunologically active and/or functional fragment thereof, is indicated by the presence of the agent in the complex.

In a further preferred embodiment, the present invention provides a method to identify candidate drugs for treating a pathological condition or a susceptibility to a pathological condition, such as tumors or wounds, comprising: contacting a test agent with cells which express one or more genes of the invention, characterized by the polynucleotides of the invention, such as TAG, GAG/A and/or GAG/B polynucleotides, and preferably selected from the group consisting of SEQ ID NO:s 1, 3, 5, 7, 9, 11, 13, 15, 17, 19, 21, 23, 25, 27, 29, 31 and 33, including parts thereof, determining the amount of expression of said one or more genes by hybridization of mRNA of said cells or cDNA or cRNA copied from said mRNA to a nucleic acid probe which is complementary to an mRNA of said one or more genes; identifying a test agent as a candidate drug for treating a pathological condition or a susceptibility to a pathological condition if it modulates the expression of said one or more genes. Preferably, the present invention relates to a method as described supra, wherein said a pathological condition or a susceptibility to a pathological condition is a tumor, and wherein said test agent is identified as a candidate drug for treating said tumor if it decreases expression of said one or more genes.

In an another embodiment, the present invention relates to a method as described above, wherein said a pathological condition or a susceptibility to a pathological condition is impaired wound healing, and wherein said test agent is identified as a candidate drug for treating said impaired wound healing if it increases expression of said one or more genes.

In an even further preferred embodiment, the present invention provides a method to identify candidate drugs for treating a pathological condition or a susceptibility to a pathological condition, such as tumors or wounds, comprising: contacting a test agent with cells which express one or more polypeptides according to the invention, such as TAG, GAG/A and/or GAG/B polypeptides, and preferably selected from the group consisting of SEQ ID NO:s 2, 4, 6, 8, 10, 12, 14, 16, 18, 20, 22, 24, 26, 28, 30, 32 and 34, including parts thereof; determining the amount of said one or more of said polypeptides in said cells; identifying a test agent as a candidate drug for treating a pathological condition or a susceptibility to a pathological condition if it modulates the amount of one or more of said polypeptides in said cells. Preferably, the present invention provides a method described above, wherein said pathological condition or a susceptibility to a pathological condition is a tumor, and wherein said test agent is identified as a candidate drug for treating said tumor if it decreases the amount of one or more of said proteins in said cells. The present invention also contemplates a method as described supra, wherein said pathological condition or a susceptibility to a pathological condition is impaired wound healing, and wherein said test agent is identified as a candidate drug for treating said impaired wound healing if it increases the amount of one or more of said proteins in said cells.

Also, the present invention provides a method for identifying candidate drugs for treating a pathological condition or a susceptibility to a pathological condition, such as tumors or wounds, comprising: contacting a test agent with cells which express one or more polypeptides according to the invention, such as TAG, GAG/A and/or GAG/B polypeptides, and preferably selected from the group consisting of SEQ ID NO:s 2, 4, 6, 8, 10, 12, 14, 16, 18, 20, 22, 24, 26, 28, 30, 32 and 34, including parts thereof; determining activity of said one or more polypeptides in said cells; identifying a test agent as a candidate drug for treating a pathological condition or a susceptibility to a pathological condition if it modulates the activity of one or more of said polypeptides in said cells. As such, the present invention particularly relates to a method as described herein, wherein said pathological condition or a susceptibility to a pathological condition is a tumor, and wherein said test agent is identified as a candidate drug for treating said tumor if it decreases the activity of one or more of said proteins in said cells.

According to a preferred embodiment, the present invention relates to a method as described herein, wherein said pathological condition or a susceptibility to a pathological condition is impaired wound healing, and wherein said test agent is identified as a candidate drug for treating said impaired wound healing if it increases the activity of one or more of said proteins in said cells.

In another embodiment, the present invention provides a method to identify candidate drugs for treating patients having pathological conditions or a susceptibility to a pathological condition, such as bearing tumors or for treating wounds, comprising: contacting a test agent with recombinant host cells which are transfected with an expression construct which encodes one or more polypeptides according to the invention, such as TAG, GAG/A and/or GAG/B polypeptides, and preferably selected from the group consisting of SEQ ID NO:s 2, 4, 6, 8, 10, 12, 14, 16, 18, 20, 22, 24, 26, 28, 30, 32 and 34, including parts thereof; determining the amount of proliferation of said cells; identifying a test agent as a candidate drug for treating patients having a pathological condition or a susceptibility to a pathological condition if it modulates proliferation of said cells. Accordingly, the present invention relates to a method of above, wherein said pathological condition or a susceptibility to a pathological condition is a tumor, and wherein said test agent is identified as a candidate drug for treating said tumor if it inhibits proliferation of said cells. Accordingly, the present invention relates to a method as described above, wherein said pathological condition or a susceptibility to a pathological condition is impaired wound healing, and wherein said test agent is identified as a candidate drug for treating said impaired wound healing if it stimulates proliferation of said cells.

It will be appreciated by the person skilled in the art, that the present invention also relates to any of the methods described herein, wherein said pathological condition or a susceptibility to a pathological condition is chosen from the group consisting of proliferative disorders, including tumors, diabetic retinopathy, rheumatoid arthritis, psoriasis, restenosis, endometriosis, impaired wound healing, and atherosclerosis. In addition, the present invention also relates to any of the methods described herein, wherein said pathological condition relates to enhancing wound healing.

The present invention also provides a method to identify a ligand involved in endothelial cell regulation, comprising: contacting an isolated and purified human polypeptide according to the invention, such as TAG, GAG/A and/or GAG/B polypeptides, and preferably selected from the group consisting of SEQ ID NO:s 2, 4, 6, 8, 10, 12, 14, 16, 18, 20, 22, 24, 26, 28, 30, 32 and 34, including parts thereof, and preferably a transmembrane polypeptide with a test compound and a molecule comprising an antibody variable region which specifically binds to said polypeptide, preferably to an extracellular domain of said transmembrane polypeptide, or a part thereof; determining the amount of binding of the molecule comprising an antibody variable region to the polypeptide, preferably a human transmembrane polypeptide; whereby a test compound which diminishes the binding of the molecule comprising an antibody variable region to said polypeptide, such as a human transmembrane polypeptide is identified as a ligand involved in endothelial cell regulation. Preferably, the method further comprises contacting the test compound with endothelial cells and determining if the test compound inhibits growth of said cells.

In the methods according to the invention the used cells may be any mammalian cell, including cultured cells, cell lines, or primary cultures such as HUVEC. Preferably, the cells are endothelial cells, including resting and activated cells. The cells may be recombinant host cells which are transfected with an expression construct encoding one or more of the polypeptides according to the invention, or the cells may be in a mammal.

It will be appreciated that the present invention relates also to the agent identified by the method as described herein, as well as a method for the production of a composition comprising the steps of admixing an agent identifiable by a method according to the invention with a pharmaceutically acceptable carrier.

### I. KITS AND COMPOSITIONS

The present invention provides kits for the diagnosis of a pathological condition related to aberrant angiogenesis in a patient, such as impaired or excessive angiogenesis.

Accordingly, in an embodiment, the present invention provides a kit for the diagnosis of a pathological condition in a patient comprising a nucleic acid or an antibody according to the invention, and possibly a manual for use. Preferably, the pathological condition to be diagnosed is a proliferative disease or disorder or impaired wound healing. As such, the present invention also pertains to the use of a nucleic acid, polypeptide or antibody, according to the invention for the preparation of a diagnostic kit, which may include a manual, for detecting a pathological condition, such as a proliferative disease or disorder and/or impaired wound healing.

It is another object of the present invention to provide a composition comprising an therapeutic agent that binds to a marker which is expressed, accessible or localized on intratumoral blood vessels of a vascularized tumor, possibly comprising an anti-cellular moiety, wherein said marker is chosen from the group consisting of a polypeptide according to the invention, such as TAG, GAG/A and/or GAG/B polypeptides, and preferably chosen from any of SEQ ID NO:s 2, 4, 6, 8, 10, 12, 14, 16, 18, 20, 22, 24, 26, 28, 30, 32 or 34, or a part thereof.

It will be understood that the following figures and examples are meant to illustrate the embodiments of the present invention and are in no way to be construed as limiting the present invention. To the contrary, the teachings of the specific examples are intended to be generalized for substantiating the embodiments. As such, the present invention may be practiced other than as particularly described and still be within the scope of the accompanying claims.

### SHORT DESCRIPTION OF THE FIGURES AND TABLES

FIG. 1: Endothelial cell selection for gene expression profiling by SSH

A) Immunohistochemical staining of normal colon, colon tumor and placenta tissues for CD31, CD34 and CD146 to determine which antigens are most specific for endothelial cell selection. Anti-CD31 and anti-CD34 antibodies specifically stain endothelial cells in all three tissues.

B) Endothelial cells selected from fresh tissues using antibodies and magnetic beads (black spots) were stained for von Willebrand factor (vWF; green) to determine purity of the selected cell population. Nuclei were counterstained with DAPI (blue).

C) RNA isolated from the selected endothelial cells shows very good integrity.

D) Section of the SSH filters hybridized with TEC, NEC and PLEC, showing several spots with overexpression in TEC.

FIG. 2: Endothelial gene expression and tissue environment.

A) Interrelationship between endothelial cells of different sources and their gene expression signatures. EC from a malignant and pro-angiogenic environment (TEC) are compared with EC from organ matched and patient matched non-malignant sources (NEC), and with non-malignant pro-angiogenic microenvironment derived EC (PLEC) to identify the subset of genes that show expression induced by the tumor microenvironment specifically (tumor EC ‘signature’ markers').

B) Venn diagram representation of upregulated genes in different types of endothelial cells. Four pair-wise comparisons were performed by cDNA array screening of SSH repertoires: tumor conditioned (HUVEC+) vs quiescent HUVEC (HUVEC−), colorectal tumor endothelial cells vs normal colon endothelial cells (TEC vs NEC), colorectal tumor endothelial cells vs placenta endothelial cells (TEC vs PLEC) and placenta endothelial cells vs normal colon endothelial cells (PLEC vs NEC). Included are spots that showed at least a 2-fold difference in expression.

C) TAG markers classified as being overexpressed in TEC vs NEC and in TEC vs PLEC (tumor EC ‘signature’ markers) are strongly biased towards genes associated with extracellular matrix remodeling.

D) GAG/A markers, classified as overexpressed in both TEC and PLEC vs NEC (angiogenesis markers) show a diverse functional profile.

E) GAG/B markers, classified as overexpressed in TEC and in activated HUVEC, are biased to protein turnover and transcriptional activity.

FIG. 3: Expression validation of TAGs.

A) Transcriptional validation of TAG markers. Shown are expression ratios in TEC vs NEC (black bars) and TEC vs PLEC (grey bars), normalized for cyclophilin A, by quantitative real-time PCR.

B) Immunohistochemical staining of different TAGs in colorectal tumor and normal colon tissue sections.

C) Relative protein expression levels of HMGB1, IGFBP7 and vimentin on tumor endothelial cells compared to normal endothelial cells, assessed by flow-cytometry.

FIG. 4: Inhibition of in vitro and in vivo angiogenesis by antibody-mediated targeting of TAG proteins.

A) Sprout formation of bovine capillary endothelial cells (BCEs) in collagen gel is inhibited by the addition of antibodies directed against different cell surface and secreted TAGs.

B) Angiogenesis in the chick chorioallantoic membrane is inhibited by treatment with antibodies directed at the indicated TAGs.

C) Capillary sprouting of tumor vessels embedded in collagen gel is inhibited by antibodies directed at the indicated TAGs.

FIG. 5: Modulation of TAG expression affects endothelial cell function

A-C) EVLC2 cells were transfected with expression constructs containing HMGB10RF in the sense (HMGB1-S) or in the antisense (HMGB1-AS) orientation, to study the influence of over-expression and down-regulation of HMGB1 on endothelial cell biology. A) HMGB1-S cells have an increased ability to migrate into a wounded area as compared to controls and HMGB1-AS cells. B) RTQ PCR analysis indicates MMP9 expression is increased in HMGB1-S cells as compared to HMGB1-AS cells. C) HMGB1-AS cells have an impaired ability to respond to growth factor activation,

D-F) Effects of siRNA mediated down-regulation of vimentin on endothelial cell biology. Down-regulation of vimentin by siRNA duplexes significantly inhibits endothelial cell migration (D) and sprouting (E), where only high concentrations of siRNA duplex inhibit cell proliferation (F).

FIG. 6: Inhibition of tumor angiogenesis on CAM by targeting TAGs

LS174T tumor cell spheroids were transplanted onto the CAM and treated with antibodies directed against HMGB1 (A) and vimentin (B). Transplantation of tumor cell spheroids induces increased vascular density and aberrant vascular morphology in the CAMs (a) as compared to normal CAMs (b). Tumor-induced vasculature was reduced by treatment with antibodies (c, f). Chicken endothelial cell reactivity of the antibodies was confirmed by immunohistochemistry using the treatment antibodies (e) and non-relevant control antibodies (d).

FIG. 7: Inhibition of tumor growth and tumor angiogenesis antibody-mediated targeting of TAGs

A) Tumor growth curves of LS174T xenografts in nude mice, treated with vehicle, isotype control antibody (10 mg/kg) or anti-vimentin antibody (10 mg/kg and 1 mg/kg). Antibodies were administered every 3 days i.p. for a period of 12 days. A dose-dependent inhibition of tumor growth is evident in mice treated with anti-vimentin antibody 1 mg/kg, **p<0.0001; 10 mg/kg, **p<0.0001; Two-way ANOVA), whereas treatment with the isotype control antibody did not show inhibition of tumor growth (IIB5 10 mg/kg, p=0.661).

B) Immunohistochemical staining of LS174T tumor xenografts in mice with CD31 (a) and anti-vimentin antibody (b) show that vimentin expression is restricted to the endothelium. Microvessel density of treated LS174T tumors was assessed by the number of pixels representative of immunoreactivity for CD31 in control mice (c), isotype control antibody treated mice (10 mg/kg/treatment) (d), anti vimentin antibody (1 mg/kg/treatment) (e) and anti vimentin antibody (1 mg/kg/treatment) treated mice (f).

C) Quantification of microvessel density (**p<0.001, Student's T-test).

D) Body weight of mice during treatment as an indicator of possible toxicity.

E) Detection of treatment antibodies targeted to the tumor endothelium. Mouse antibodies were detected (green fluorescence) in mice treated with saline (a), isotype control antibody (b), anti-vimentin antibody (1 mg/kg/treatment) (c), and anti-vimentin antibody (10 mg/kg/treatment). Endothelial cells are stained with PE-labeled anti-CD31 antibody (red fluorescence). Localization of injected antibody to the tumor vasculature is indicated by yellow fluorescence.

FIG. 8: Diversity of expression patterns in TEC, NEC and PLEC.

Hierarchical clustering analysis of expression ratios of the entire SSH repertoire. The dendrogram represents the results of hierarchical clustering analysis based on similarities in gene expression patterns of the different comparisons indicated to the right of the clustered image maps. Expression ratios are color-coded as indicated on the far right and shown for the indicated comparisons. Bars at the bottom indicate clustered regions containing the genes that confer a tumor ‘signature’ to EC (TAGs). The dendrogram at the left is an indicator of overall correlation between the comparisons shown in the rows. Note that differential gene expression during physiological angiogesis (PLEC vs NEC) is most closely related to differential gene expression in activated vs quiescent HUVEC (H+ vs H−).

FIG. 9: TAG sequences

A) Nucleotide and amino acid sequences of TAG genes.

B) Nucleotide sequences of SSH identified TAG inserts

Table 1: Characteristics of EC gene expression identified by differential screening of SSH repertoires.

Table 2: Tumor angiogenesis genes (TAGs).

Table 3: General angiogenesis genes (GAG/A and GAG/B).

Table 4: Amplification primers for TAGs.

### EXAMPLES

### Example 1

### Experimental Procedures

1.1 Isolation of Endothelial Cells from Fresh Tissues

Fresh colorectal tumors (Dukes C) (n=5) and distant normal colon tissues of the same patient (n=5) were obtained from excision surgery at the department of Pathology (University Hospital Maastricht). Fresh placenta tissues (n=5) were obtained from the department of Obstetrics (University Hospital Maastricht). Endothelial cells were isolated as previously described (St Croix et al., 2000), with minor modifications. Tissues were minced with surgical blades, digested for 30 minutes with 1 mg/ml collagenase (Life Technologies, Breda, The Netherlands) and 2.5 U/ml dispase (Life Technologies) at 37° C. with continuous agitation. DNAse I (Sigma, Zwijndrecht, The Netherlands) was added to a final concentration of 100 μg/ml and the cell suspension was incubated for another 30 minutes prior to Ficoll Paque gradient density centrifugation (Amersham Biosciences, Uppsala, Sweden).

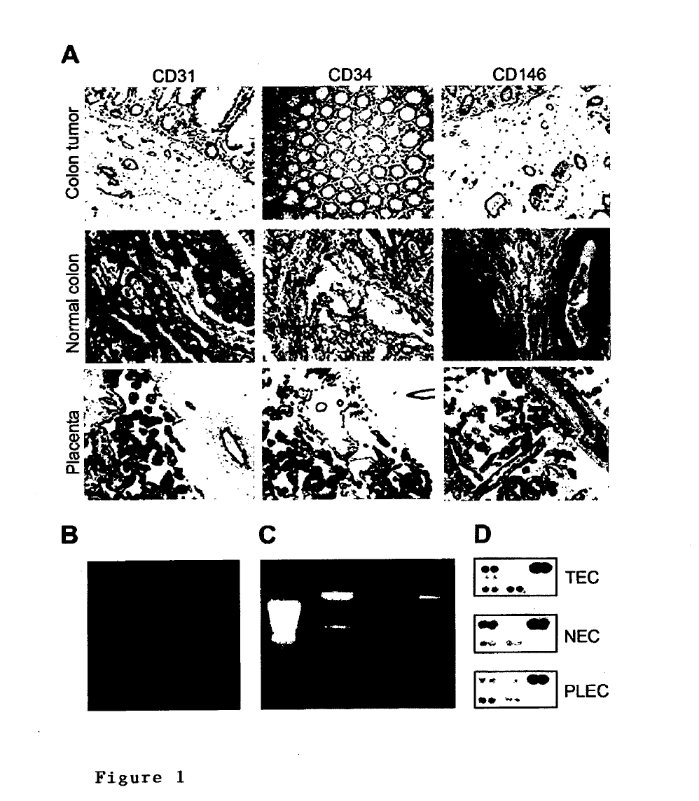

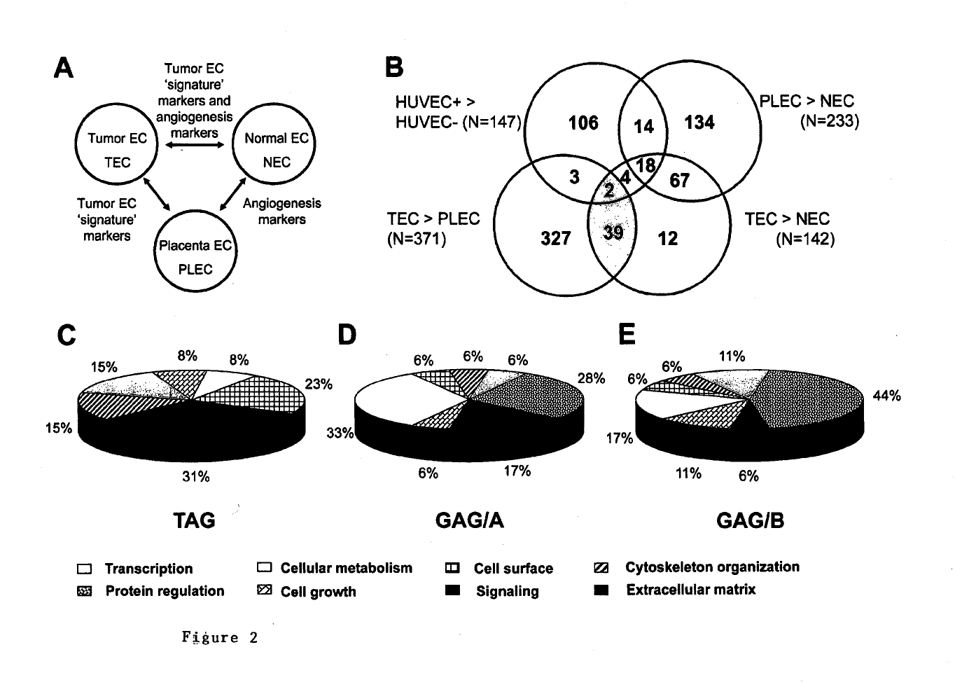

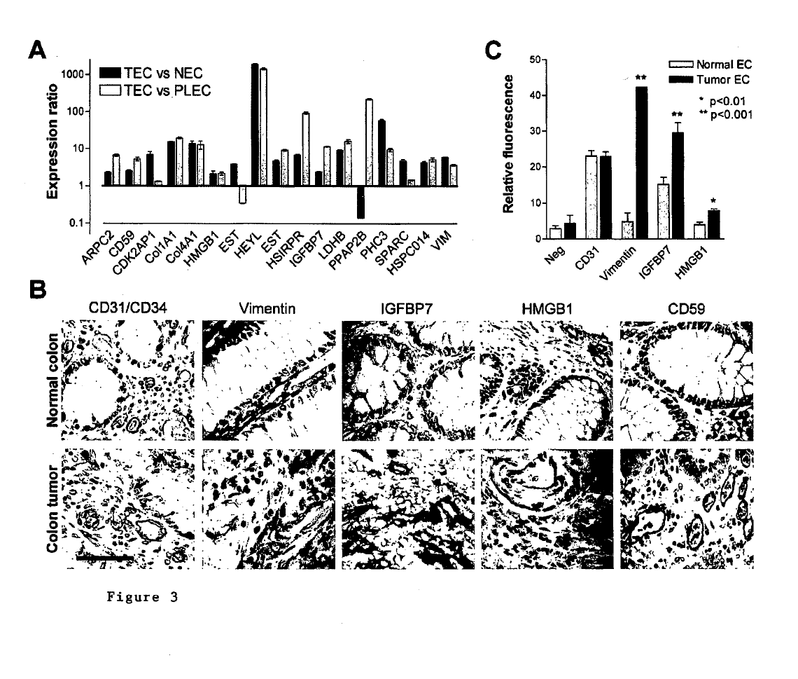

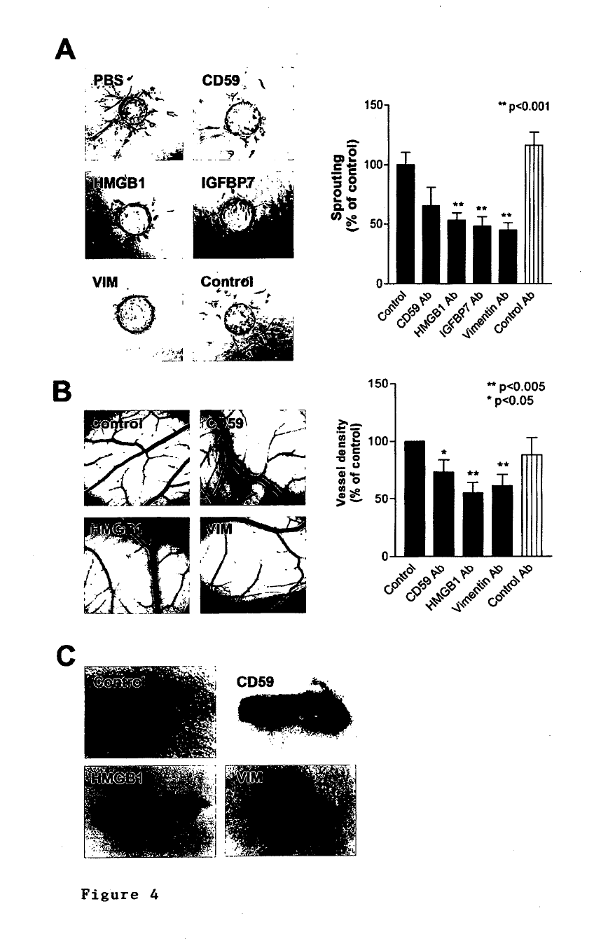

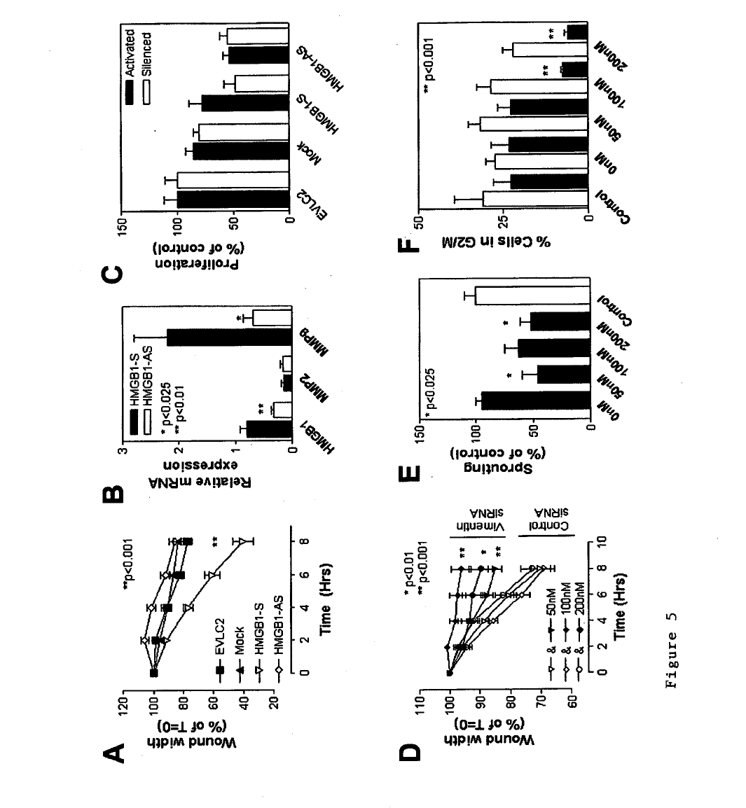

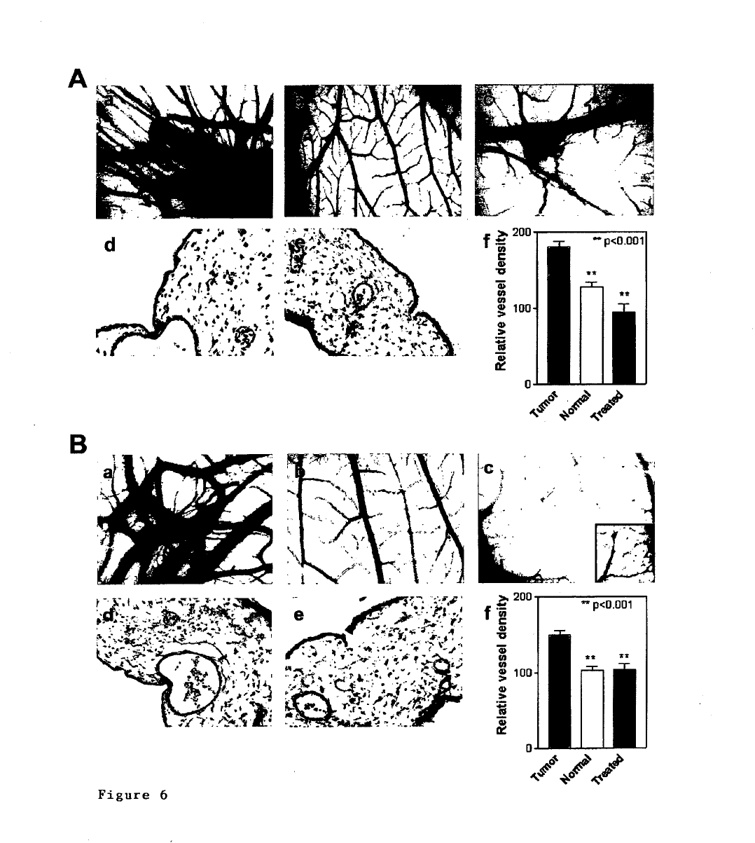

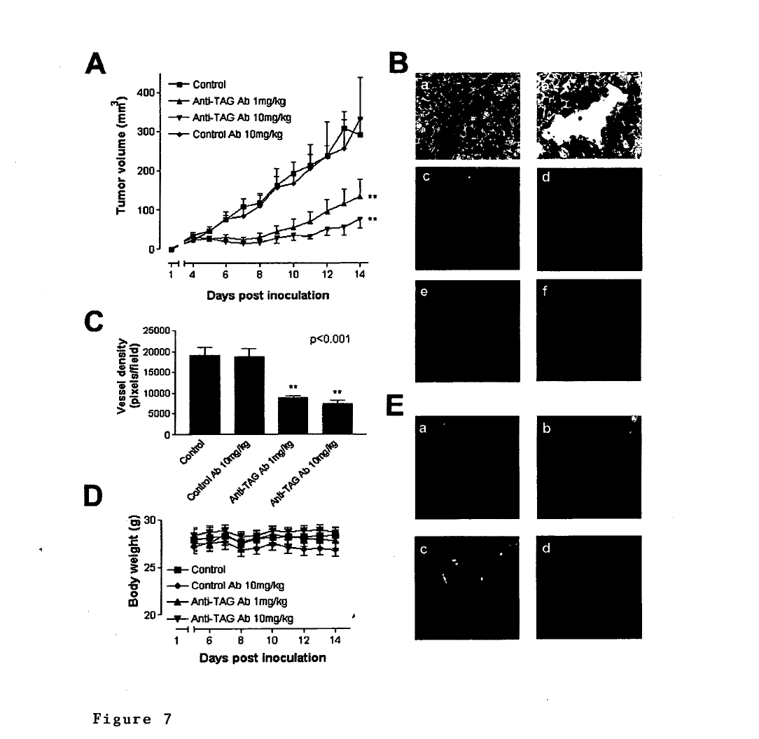

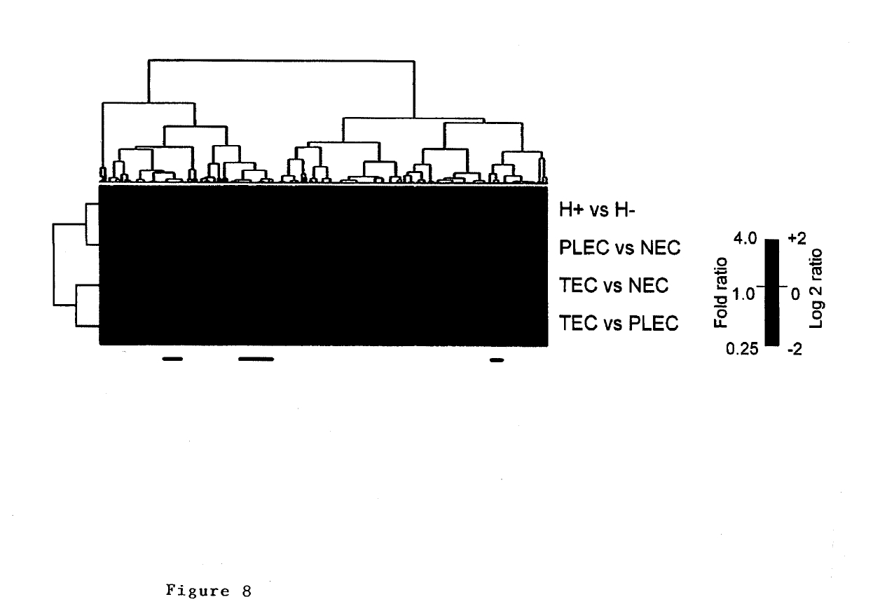

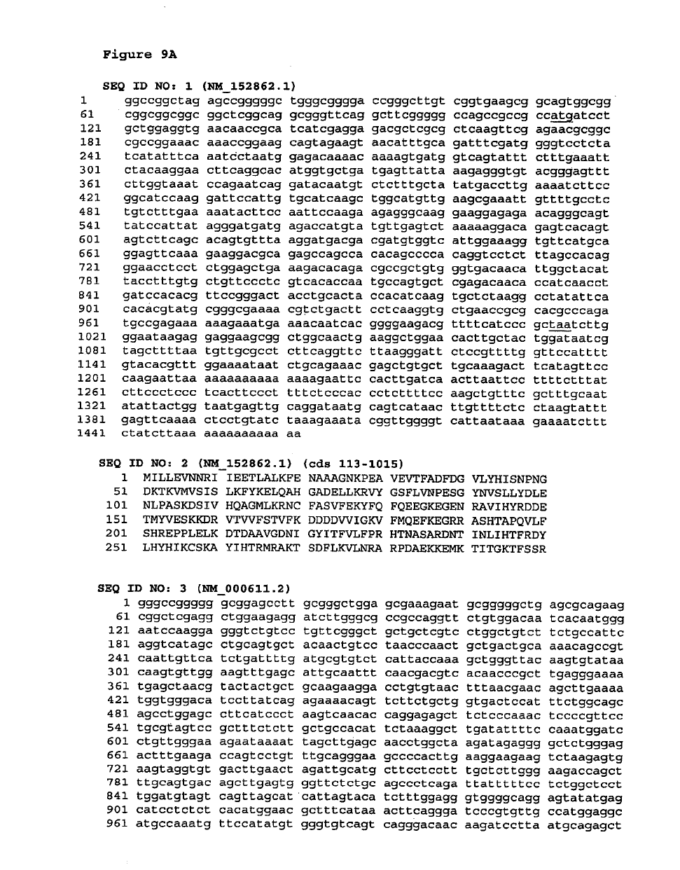

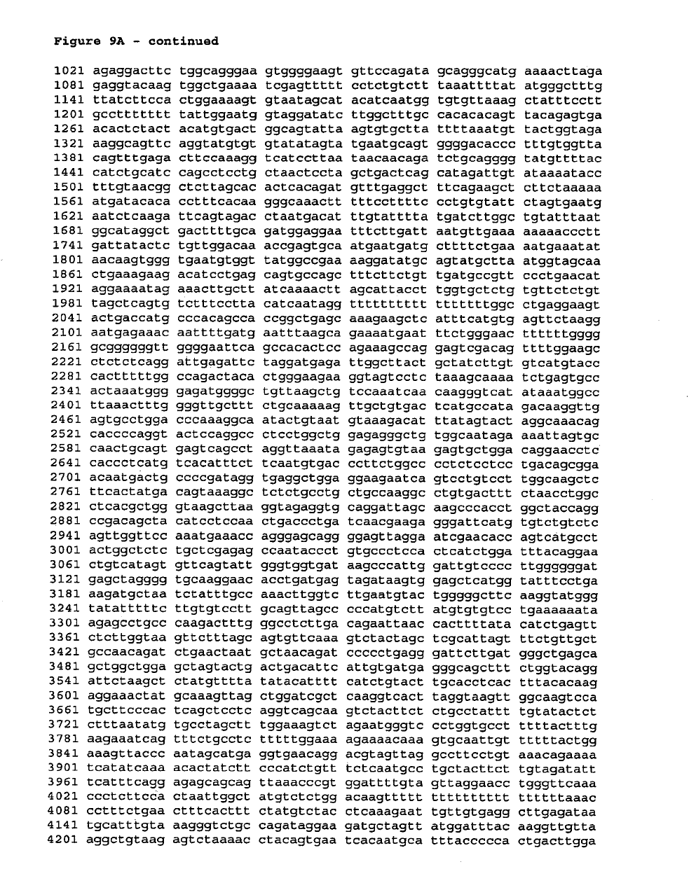

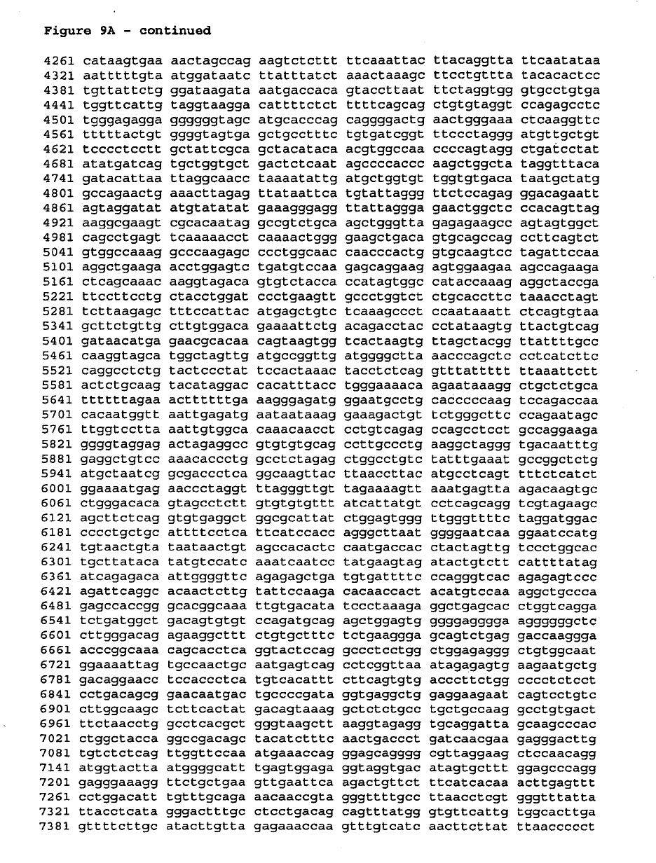

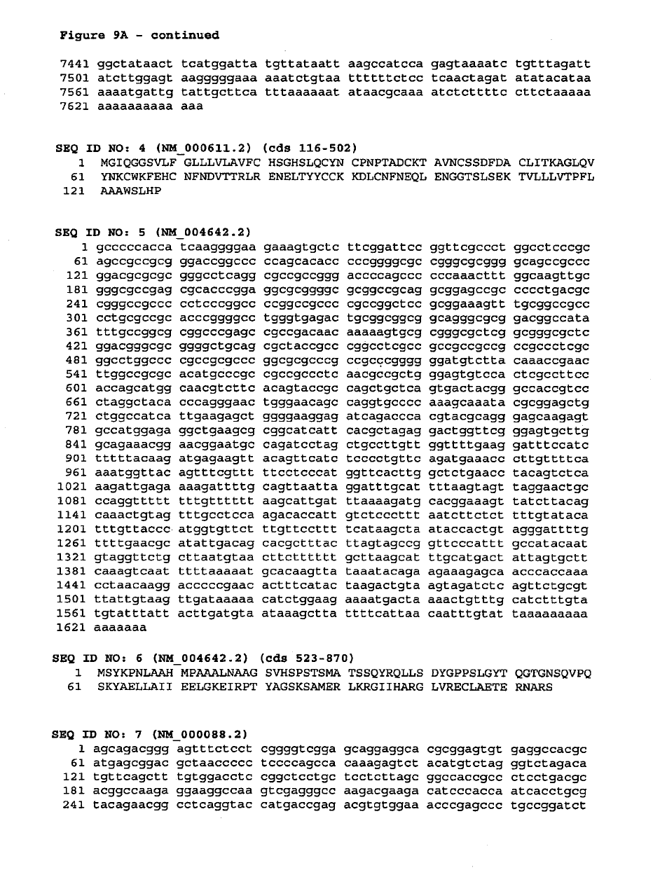

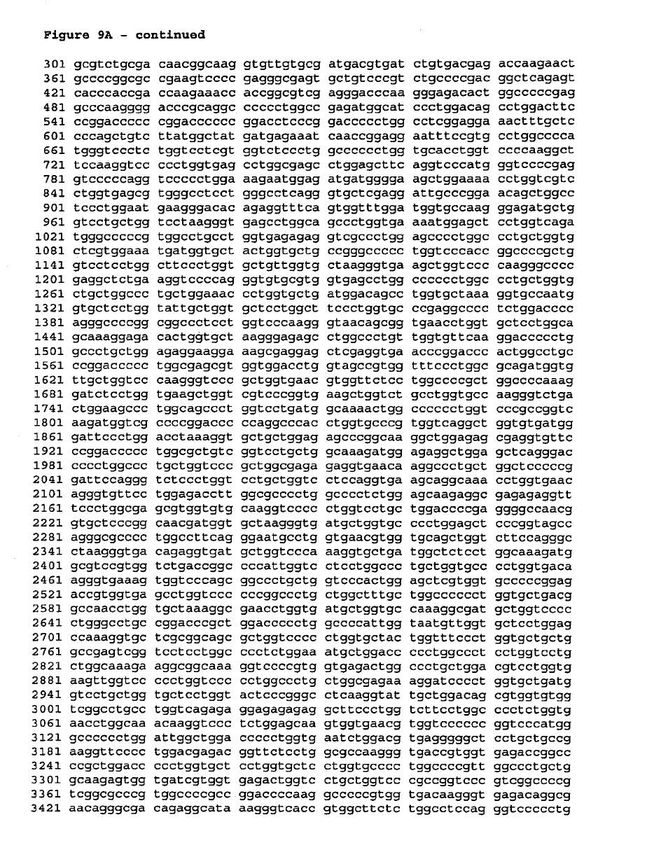

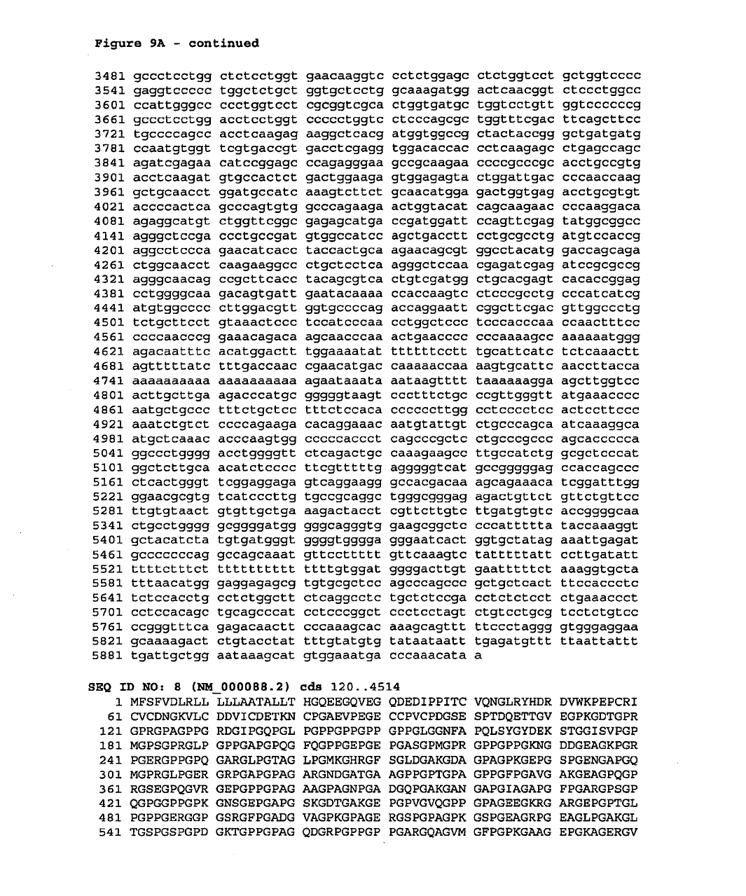

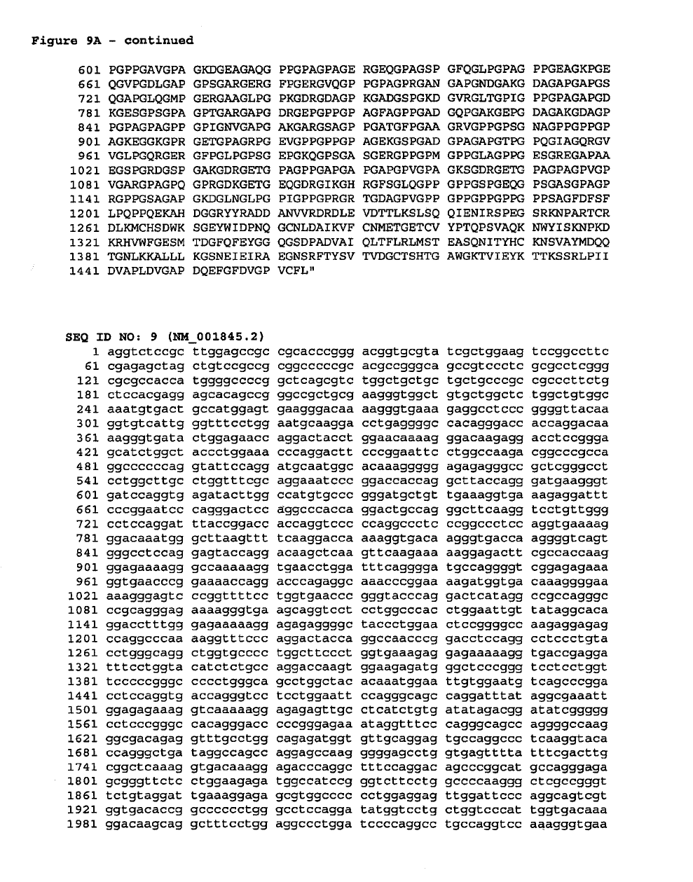

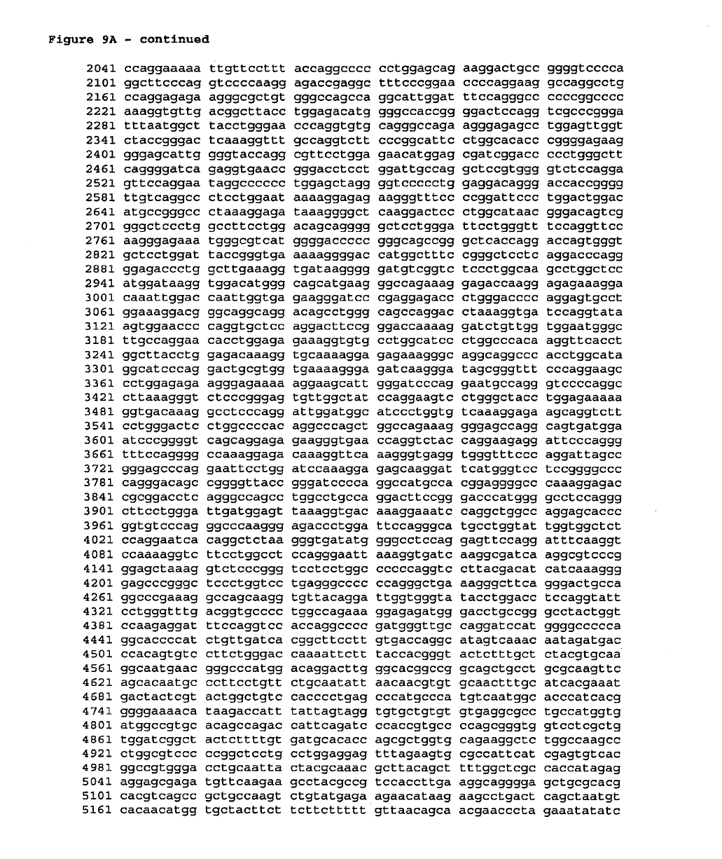

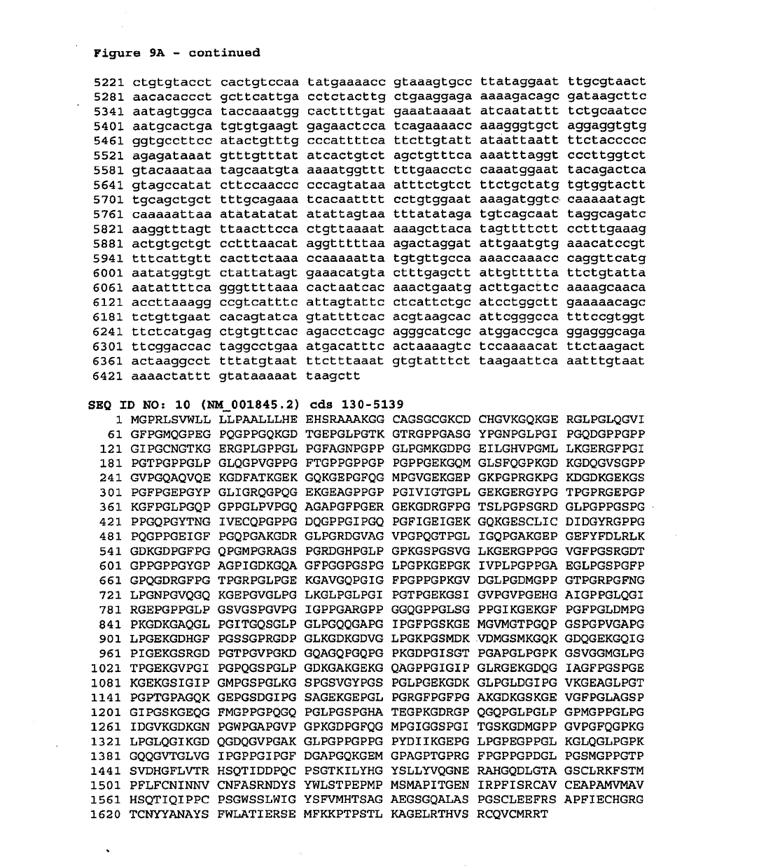

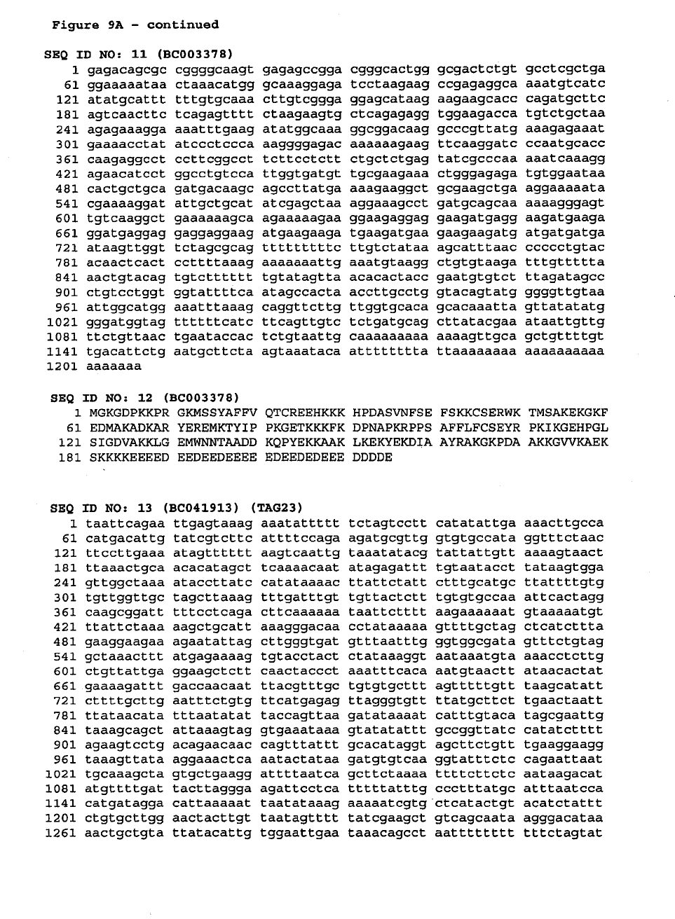

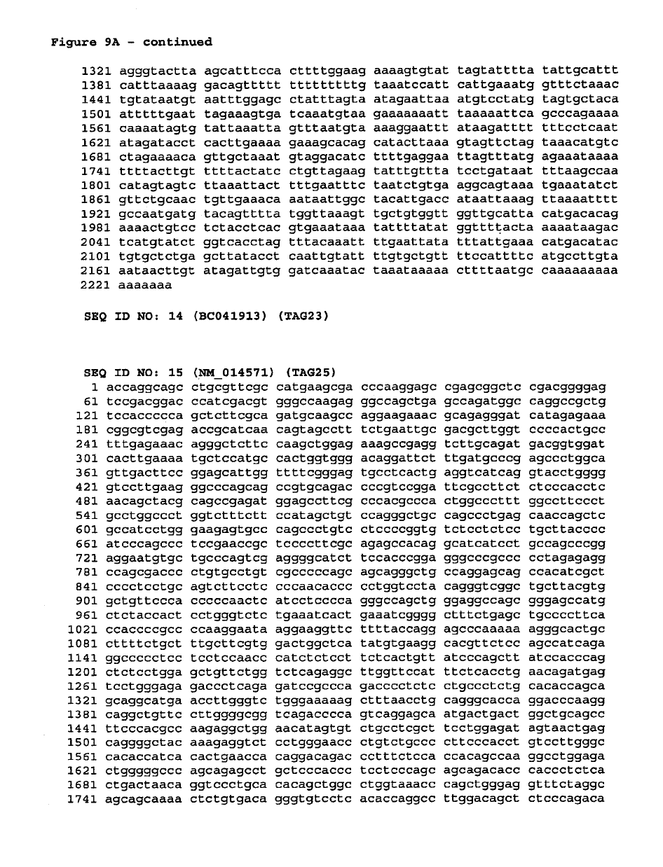

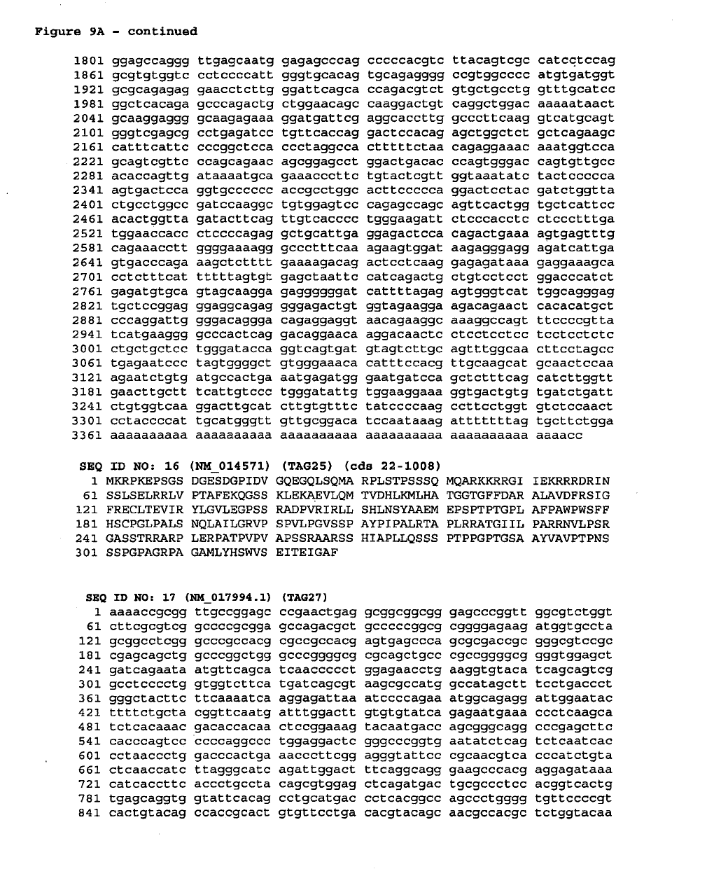

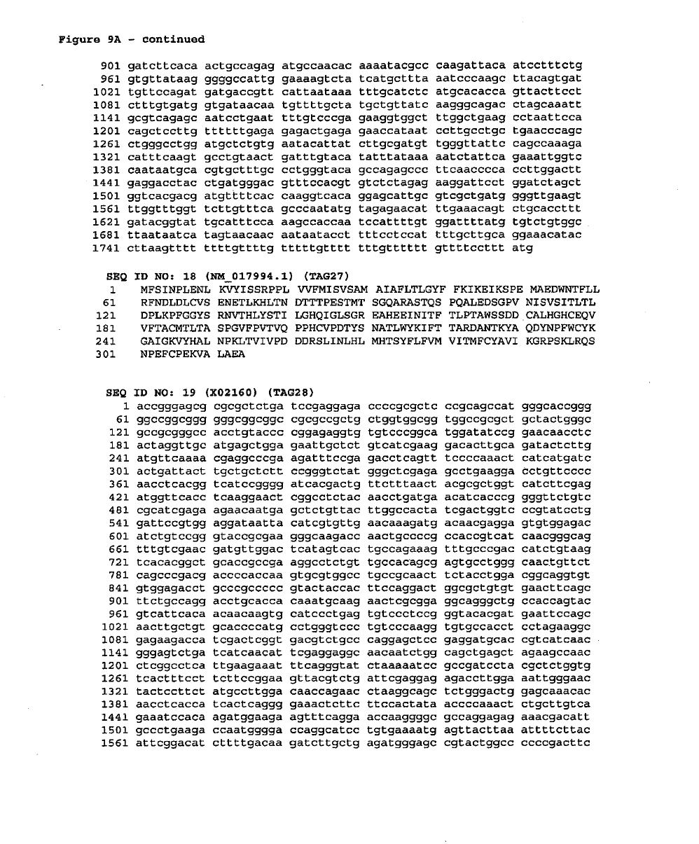

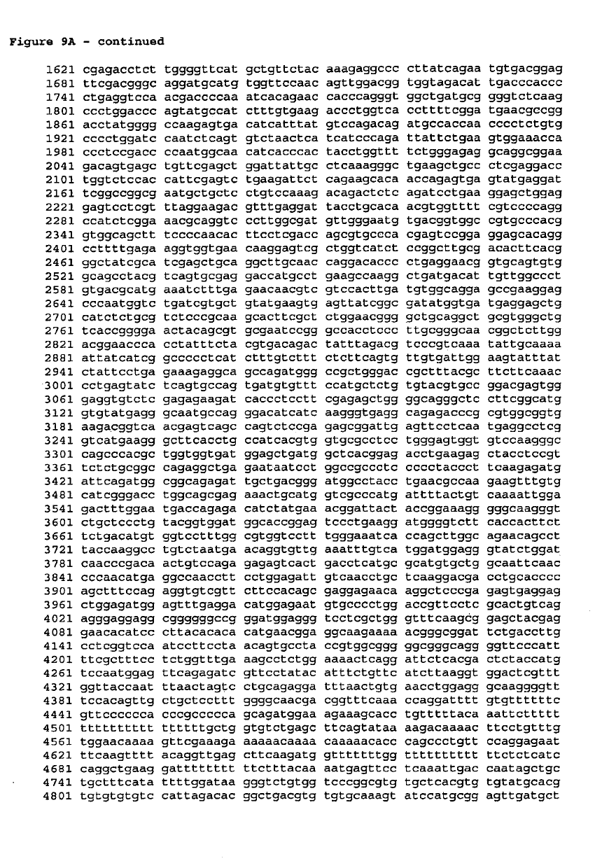

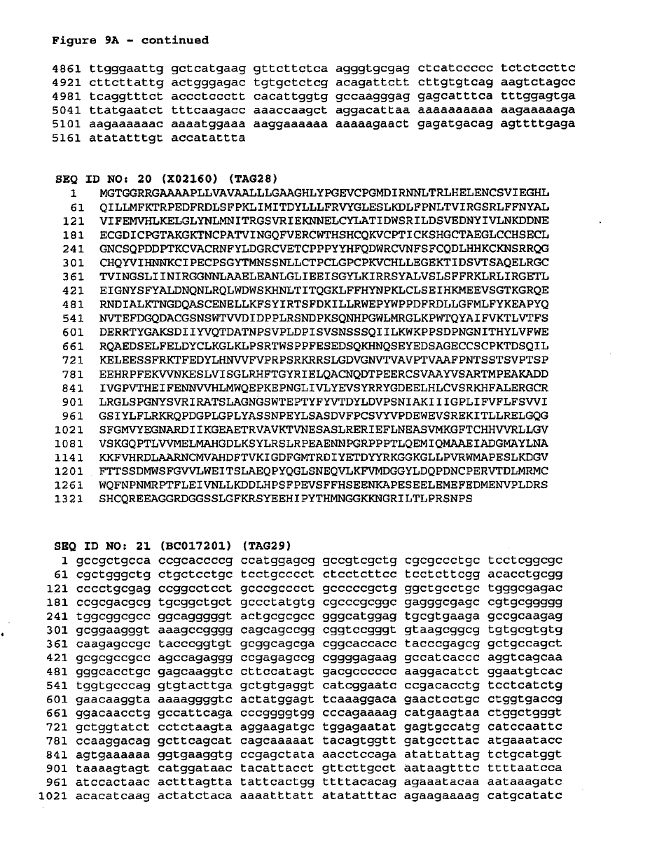

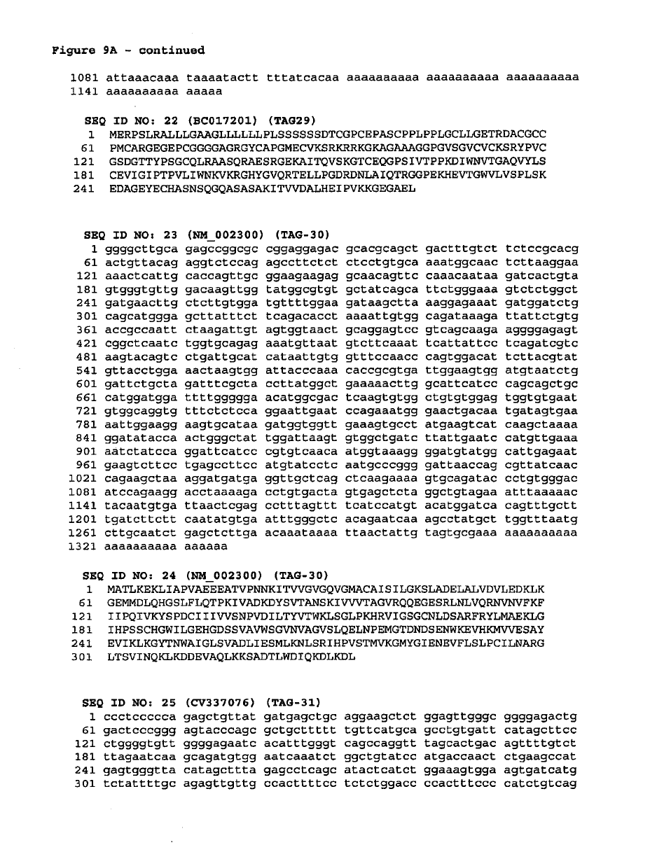

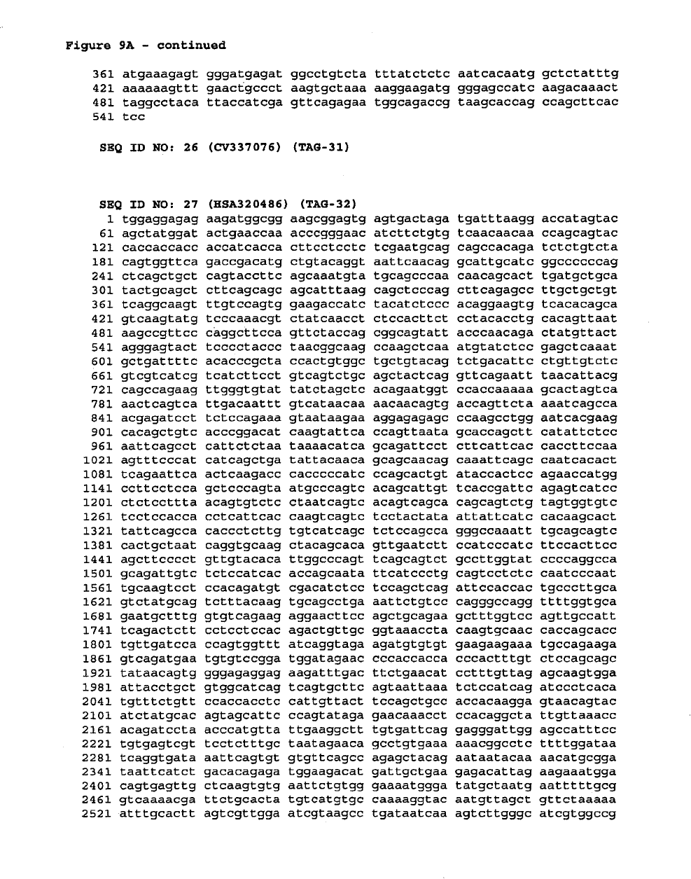

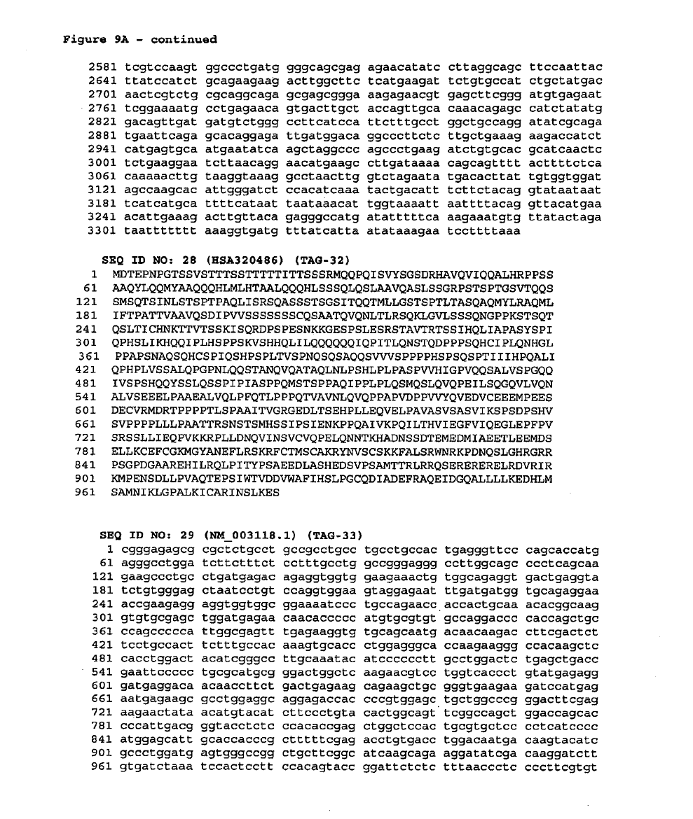

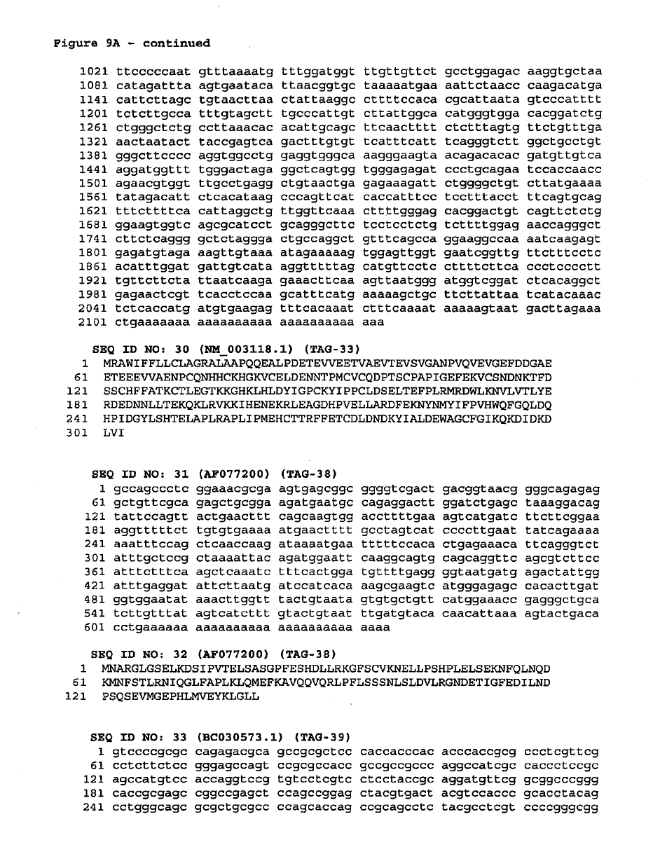

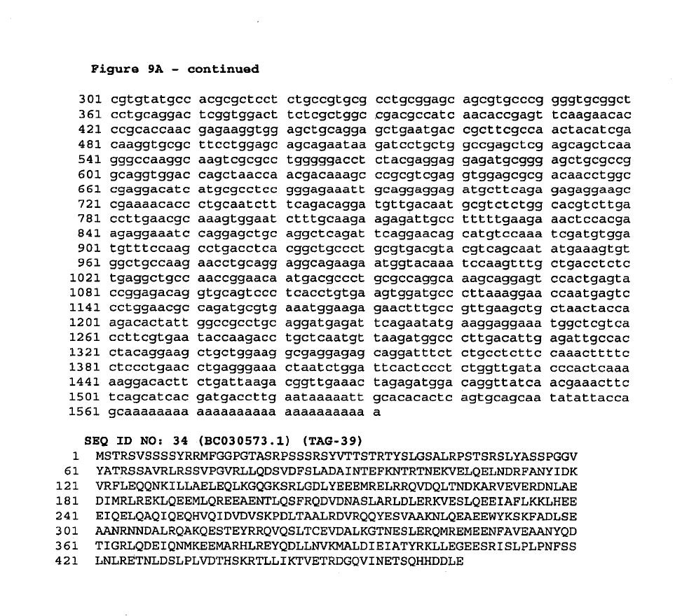

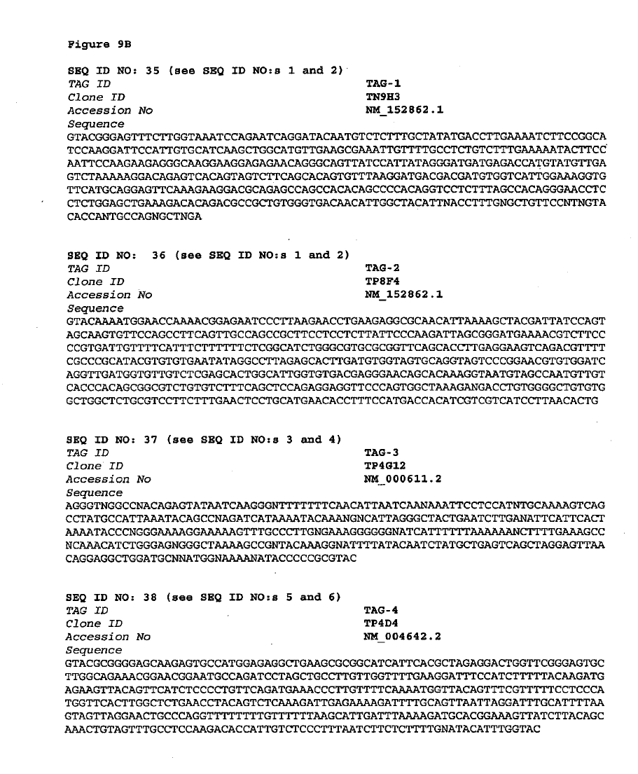

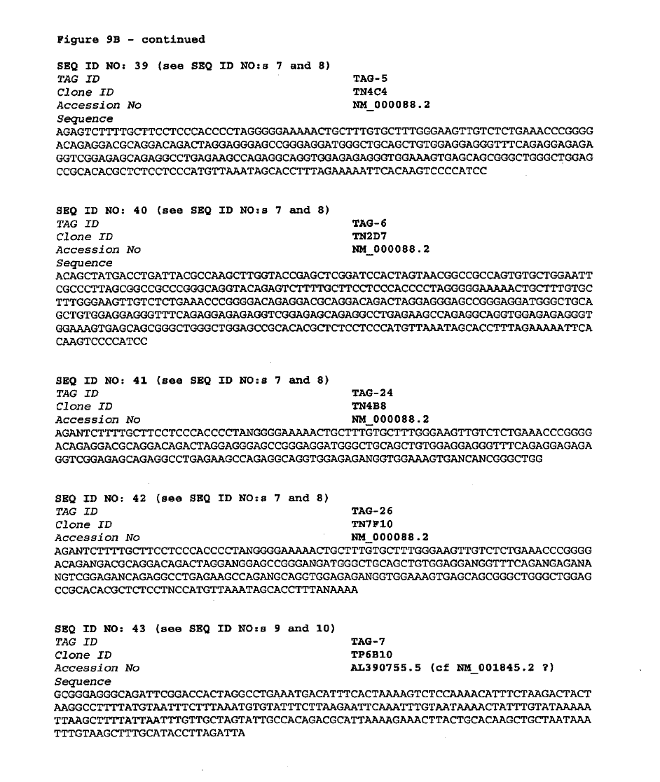

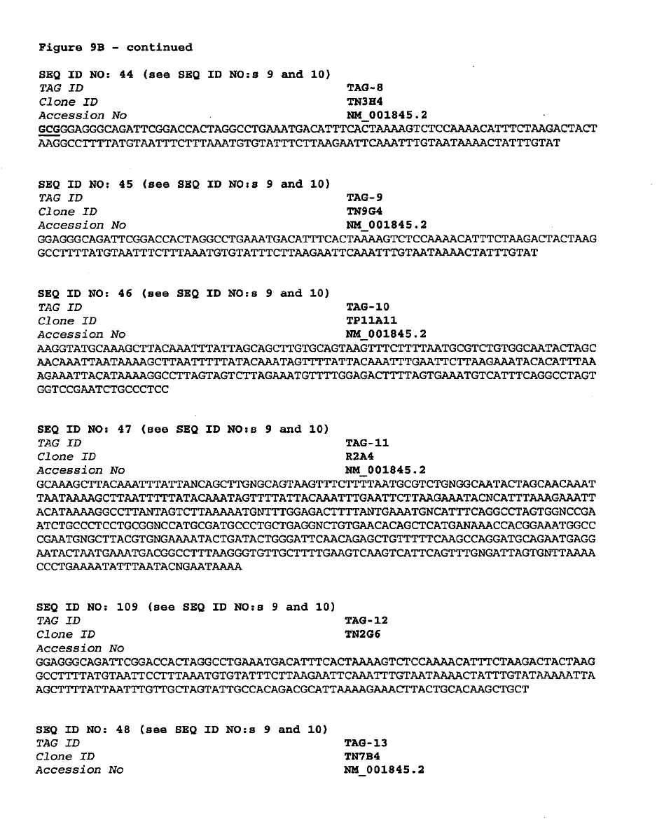

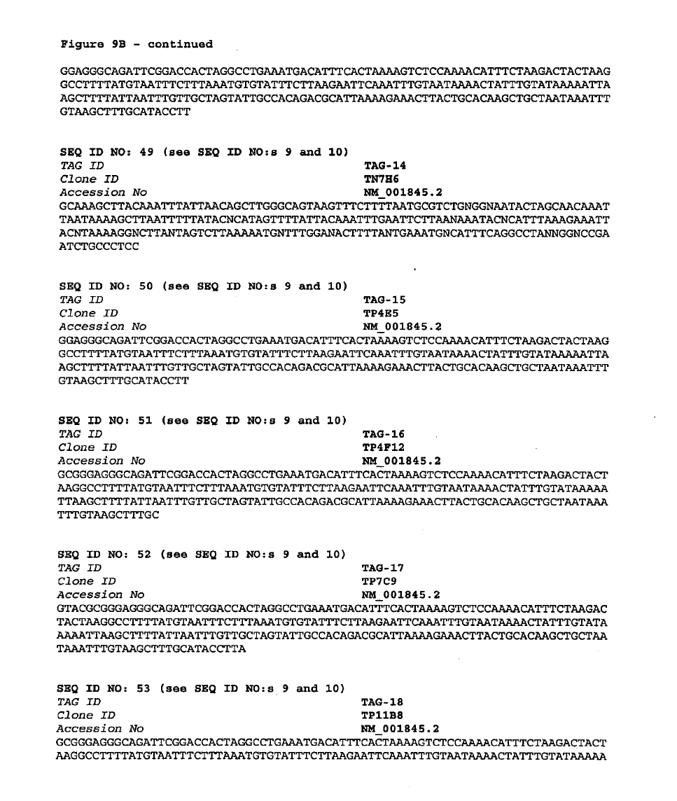

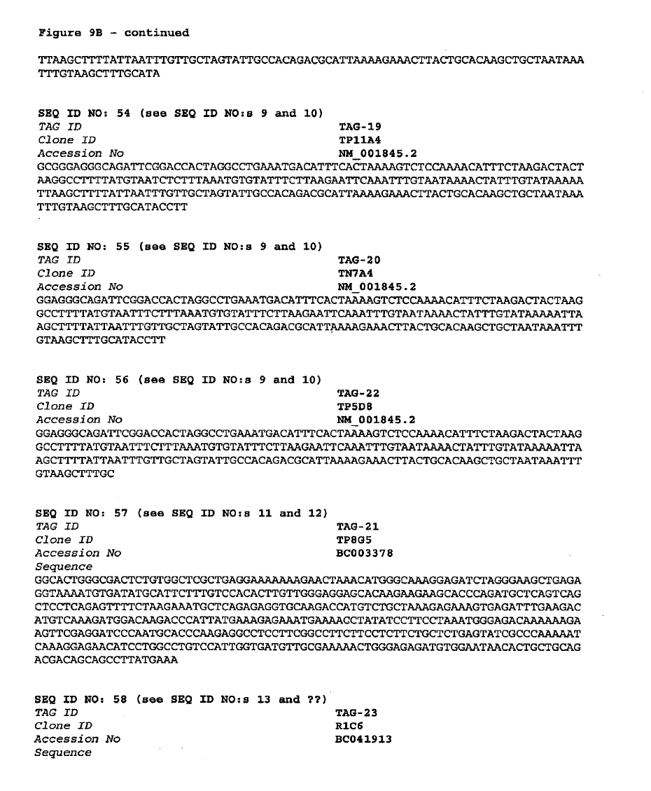

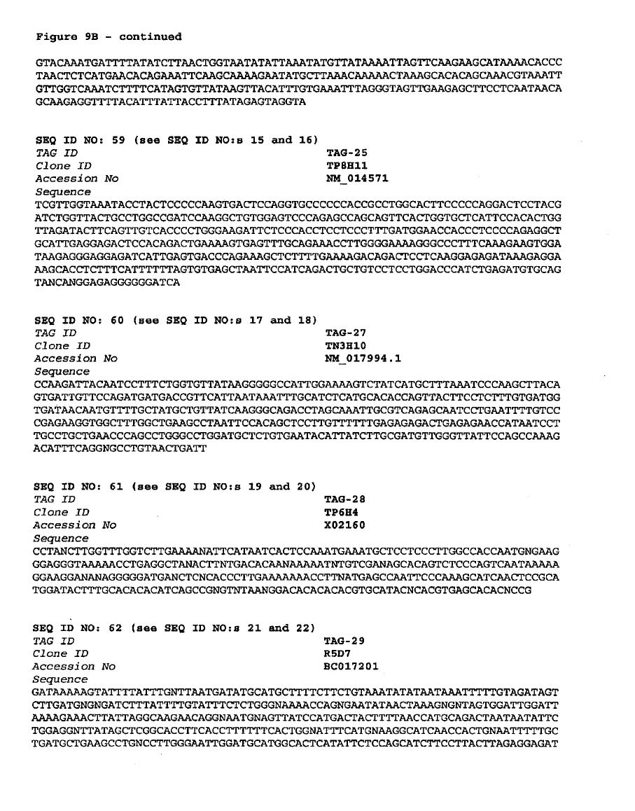

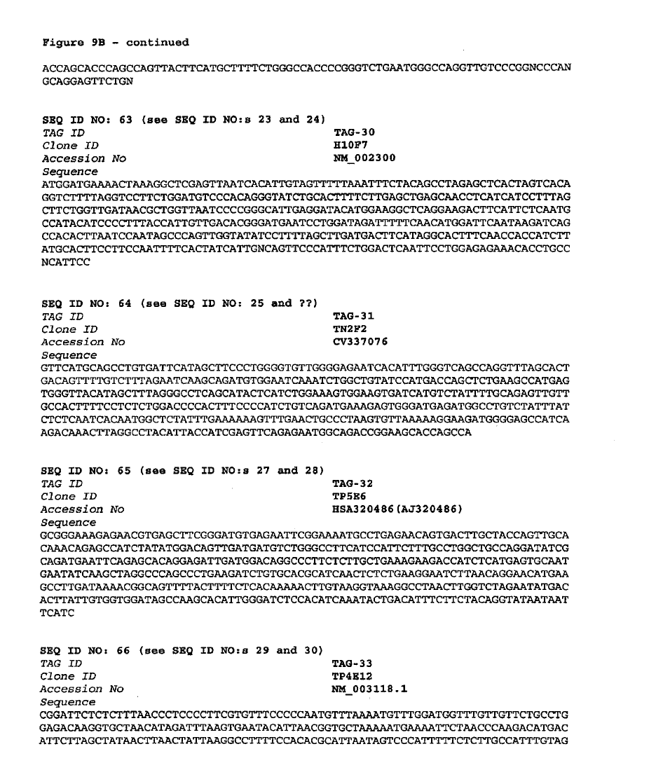

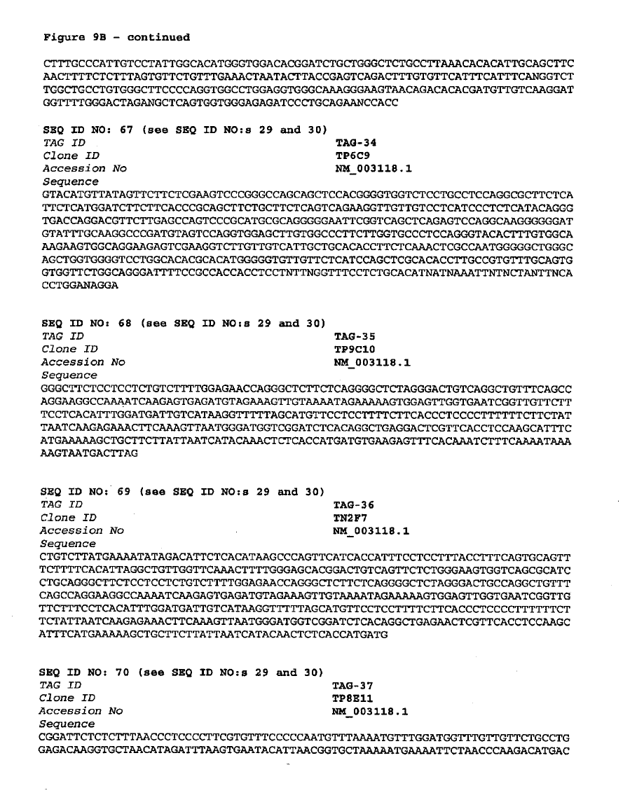

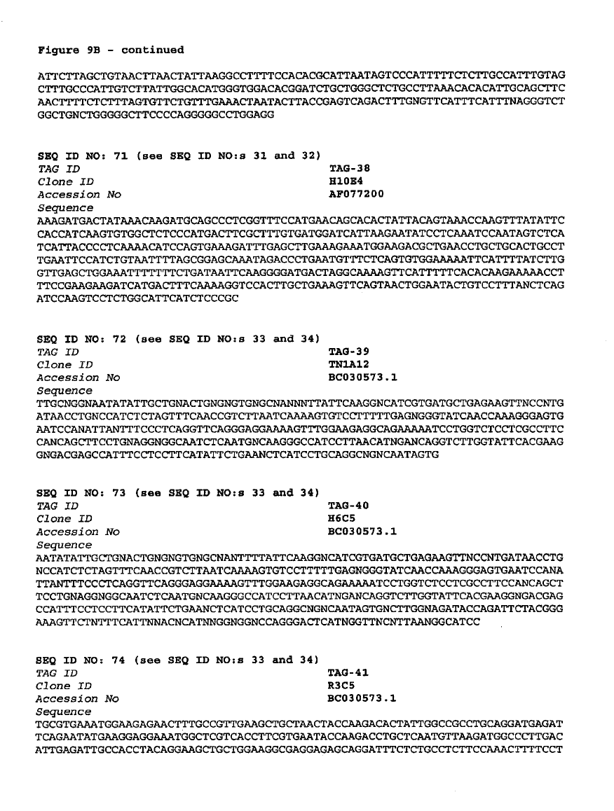

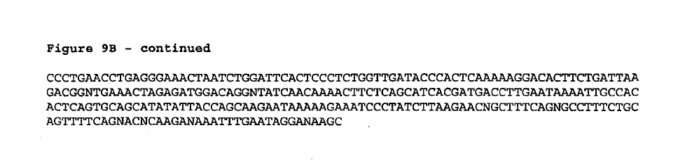

Endothelial cells were stained with anti-CD31 (clones JC/70A, DAKO, Glostrup, Sweden; and EN4, Monosan, Uden, The Netherlands) and anti-CD34 antibodies (clone Qbend10, Novocastra, Newcastle upon Tyne, United Kingdom) and isolated by positive selection using goat anti-mouse IgG coated paramagnetic beads (Dynal, Oslo, Norway). Hence, the present invention also provides for the selection of endothelial cells from human tissues for the purpose of gene expression by using the combination of CD31 and CD34. The purity of the isolated endothelial cell fraction was assessed by immunofluorescence staining for the endothelium specific von Willebrand Factor (vWF) (DAKO), and was estimated to be over 97% (FIG. 1).

### 1.2 Cell Culture

Human umbilical vein endothelial cells (HUVEC) were isolated and cultured as previously described (van der Schaft et al., 2000). HUVEC between passages 1 and 3 were used for all experiments. For experiments that required ‘tumor-activated’ endothelial cells, HUVEC were seeded in 75 cm² tissue flasks coated with 1 mg/ml fibronectin at a density of 1*10⁵ cells per flask. The cells were cultured in RPMI 1640 (Life Technologies) supplemented with 20% human serum (HS), 10% filter-sterile conditioned medium from LS174T colorectal tumor cell line, 10% filter-sterile conditioned medium from Caco-2 colorectal tumor cell line, 2 mM L-glutamine (Life Technologies), 50 ng/ml streptomycin (MP Biomedicals, Amsterdam, The Netherlands), 50 U/ml penicillin (MP Biomedicals), 1 ng/ml bFGF (Reliatech, Braunschweig, Germany) and 10 ng/ml VEGF (Reliatech) until 80% confluence was reached. ‘Quiescent’ endothelial cells were obtained by growing HUVEC for 72 hrs in fibronectin coated 75 cm² tissue flasks seeded at a density of 7*10⁵ cells per flask in RPMI 1640 supplemented with 2% HS, 2 mM L-glutamine, 50 ng/ml streptomycin, and 50 U/ml penicillin.

Alternatively, established HUVEC cell lines were used, such as the EC line EVLC2, which is a cell line derived from human umbilical vein ECs by immortalization with simian virus 40 large T antigen (Leeuwen at al., 2001).

1.3 RNA Isolation and cDNA Synthesis

RNA was isolated using RNeasy Mini reagents (Qiagen, Venlo, The Netherlands) according to the manufacturer's instructions. RNA samples were pooled for 5 colorectal tumor endothelial cell fractions (TEC), 5 normal colon endothelial cell fractions (NEC) and 5 placenta endothelial cell fractions (PLEC) and SMART™ cDNA (BD Biosciences, Alphen aan den Rijn, The Netherlands) was synthesized from the RNA and amplified to be used for SSH. The number of PCR cycles performed was optimised to maintain the original representation of transcripts in each sample. Input RNA in the cDNA synthesis reactions varied from 100 ng (isolated endothelial cells) to 1 μg (HUVEC) (FIG. 1).

### 1.4 Suppression Subtractive Hybridisation (SSH)

SSH was performed with the PCR-Select™ cDNA subtraction kit (BD Biosciences) according to the manufacturers' instructions. Subtractions were performed to create cDNA repertoires enriched for genes overexpressed in TEC and for genes differentially expressed in activated and quiescent HUVEC. Subtracted cDNA repertoires were T/A cloned in pCR2.1 (Invitrogen, La Jolla, Calif.) and introduced in TOP10 cells, according to the manufacturers' instructions. Individual colonies were picked and grown overnight at 37° C. in 2×TY bacterial medium (BD Biosciences) supplemented with 10 μg/ml ampicilin (Roche Applied Science, Almere, The Netherlands) and subsequently stored at −80° C. in 15% glycerol.

### 1.5 Differential Screening

Inserts were amplified using the adaptor specific primers Nested 1 and Nested 2R (BD Biosciences) using HotGoldstar Taq polymerase (Eurogentec, Liege, Belgium). PCR products were spotted in duplicate onto nylon membranes (Eurogentec) and hybridised to radioactively labelled cDNA probes derived from TEC, NEC, PLEC, activated and quiescent HUVEC. Approximately 100 ng of SMART™ cDNA was labeled using High Prime labelling mix (Roche) in the presence of 25 μCi ³³P-dCTP (Amersham). Membranes were pretreated with 0.6M NaCl/0.4M NaOH and subsequently prehybridised for at least 3 hours at 65° C. in 5×SSPE, 10×Denhardts solution 0.5% SDS (Roche) and 100 μg/ml salmon testes DNA (Sigma). Labelled probe was added to the hybridisation solution to an activity of 2-5*10⁶ cpm/ml and hybridised overnight at 65° C. in a roller bottle hybridization oven (Techne; Jepsons Bolton, Watford Herts, UK). Membranes were washed with increasing stringency in SSPE/SDS solutions, wrapped in saran wrap and exposed to phosphor screens (Kodak, Rochester, N.Y.) for 16-40 hours. Images were acquired using the Personal FX phosphorimager (Bio-Rad, Veenendaal, The Netherlands) at a resolution of 50 μm and analysed as Tiff files using Quantity One software (Bio-Rad). All experiments were performed two times.

Data was processed in MS Excel to identify differentially expressed transcripts. Pair-wise comparisons were performed between duplicate filters hybridised with different probes. Duplicate spots showed excellent concordance (R²>0.99, data not shown) and were averaged. Average spot intensities were included in the analysis when expression was at least 2.5 times background in any experiment. Spot intensities were normalized for total intensity of the filters under comparison. Gene expression ratios were calculated using the average normalized intensities for each spotted insert cDNA. Hierarchical clustering analysis was performed with Cluster 3.0 (de Hoon et al., 2004) and visualized using TreeView (Michael Eisen, University of California at Berkeley, Calif.).

### 1.6 Sequencing and Database Searching

Plasmid DNA was isolated using the GenElute Plasmid Miniprep kit (Sigma-Aldrich, St Louis, Mo.) and used as template for cycle sequencing. Reactions were performed using 300 ng plasmid DNA in BigDye™ Terminator Cycle Sequencing mix (Applied Biosystems, Foster City, Calif.) using M13 universal primers (Sigma Genosys, The Woodlands, Tex.) and analysed on a 3100 Genetic Analyzer (Applied Biosystems; Genome Center Maastricht, Maastricht University, The Netherlands). Homology searches were performed using NCBI nucleotide-nucleotide Blast (blastn) algorithm on the combined GenBank/EMBUDDBJ non-redundant (nr) and expressed sequence tags (est) databases (http://www.ncbi.nlm.nih.gov/blast/).

### 1.7 Real-Time Quantitative PCR(RTQ-PCR)

SYBR green assays were performed using 10 ng cDNA template per reaction, consisting of 1×SYBR Green Master Mix (Applied Biosystems) and 200 μM of each primer (Sigma Genosys) (Supplementary Table 1). Reactions were run and analysed on the ABI7700 (Applied Biosystems) using the following cycling conditions: 50° C. for 2 minutes, 95° C. for 10 minutes and 40 cycles of 95° C. for 15 seconds and 60° C. for 1 minute. All reactions were performed in triplicate, analysed using SDS software (Applied Biosystems) and further processed in MS Excel. All experiments were normalized for cyclophilin A transcript expression to account for variations in template input.

### 1.8 FACS Analysis

Single cell suspensions of fresh colorectal tumor and normal colon tissues were obtained as described above and fixed in 1% paraformaldehyde (Merck). Endothelial cells were stained with a PE-labelled anti-CD31 antibody (DAKO) and separated from other cells by cell sorting (BD FACSAria, BD Biosciences). CD31 positive cells were subsequently stained using the following antibodies, diluted in PBS, 0.5% BSA: rabbit anti-vimentin, rabbit anti-IGFBPrP1 and rabbit anti-HMGB1 followed by biotinylated swine anti-rabbit IgG (DAKO) and streptavidin-FITC (DAKO).

### 1.9 Immunohistochemistry

Formalin-fixed, paraffin embedded or snap frozen colorectal tumor and normal colon tissues were obtained from the department of Pathology (University Hospital Maastricht) and 5 μm sections were mounted onto microscope slides (Knittel, Braunschweig, Germany). Sections were deparaffinized and rehydrated in a series of xylol and ethanol where applicable and fixed with 1% paraformaldehyde (Merck, Darmstadt, Germany). Endogenous peroxidase activity was blocked using 0.3% H₂O₂ (Merck) in PBS and non-specific binding was blocked with 1% BSA (Sigma) in PBS. Antibodies were diluted in 0.5% BSA in PBS.

Colon tumor and normal colon tissue sections were stained with the following antibodies: mouse anti-human CD31 (clone JC70/A, DAKO), mouse anti-human vimentin (clone V9, DAKO), mouse anti-human CD59 (clone MEM-43, Chemicon, Temecula, Calif.), rabbit anti-human HMGB1 (kind gift of Dr. R. G. Roeder, The Rockefeller University, New York, N.Y.), and rabbit anti-human IGFBP1-rP1/IGFBP7 (kind gift of Dr. R. Rosenfeld, Oregon Health and Sciences University, Portland, Oreg.). Primary antibodies were detected with peroxidase conjugated rabbit-anti-mouse IgG (DAKO) or goat-anti-rabbit IgG (DAKO). Color was developed using DAB according to standard protocols.

### 1.10 In Vitro Sprouting

Sprouting and tube formation of ECs were studied with the use of cytodex-3 beads overgrown with ECs in a 3-dimensional gel, as described previously (van der Schaft et al., 2000). Antibodies dialyzed to PBS were added to the collagen gel and overlay medium in the described concentrations. Cells were incubated 48 hrs, after which photographs were taken of the beads. Five concentric rings were projected over the photographs, and the number of intersections of rings and sprouting endothelial cells was determined and used as a measure of in vitro tube formation.

Alternatively, tumor blood vessels from fresh colon tumors were prepared free from the surrounding tissue and sections of 1-2 mm in length were embedded in the collagen gel. Sprouting was allowed to proceed for 5-7 days.

### 1.11 Proliferation Assay

5*10³ cells were seeded in 96-well cell culture plates coated with 0.2% gelatin (Merck) and allowed to adhere for 2 hours. Antibodies, dialysed to PBS to remove traces of azide, were added to the culture medium in the indicated concentrations. Cells were cultured for 72 hours. During the last 6 hours of the assay, the culture was pulsed with 0.5 μCi [methyl-³H]-thymidine (Amersham) per well. Activity was measured using liquid scintillation counting (Wallac LSC; PerkinElmer, Boston, Mass.).

### 1.12 Endothelial Cell Migration

5*10³ cells were seeded in individual wells of gelatin coated 96-well cell culture plates and grown to confluence. Using a blunt pipette tip, a cross-shaped wound of approximately 750 μm wide was made in the well. Cells were washed with PBS, and fresh medium was added. Where appropriate, dialysed antibody was added to the medium in the indicated concentrations. Wound width was measured in 4 predefined locations per well at T=0, 2, 4, 6 and 8 hours.

### 1.13 Cell Cycle Analysis

Cells, including floating cells, were harvested and fixed in 70% EtOH. Cells were resuspended in DNA extraction buffer (45 mM Na₂HPO₄.2H₂O, 2.5 mM citric acid, 0.1% Triton-X100, pH7.4) (Merck) and incubated at 37° C. for 20 minutes. Propidium iodide (Merck) was then added to a final concentration of 2 μg/ml. Based on DNA content and scatter, cells were classified as dividing (G2/M phase), resting (G1/G0), apoptotic or necrotic.

### 1.14 RNA Interference

SiRNA duplexes were obtained from Eurogentec (Liege, Belgium), targeting the TAG at issue as well as a negative control. Cells were transfected using JetSi-ENDO (Eurogentec) according to the manufacturers' instructions. Briefly, 2500 HUVEC were seeded in a gelatin coated 96-well cell culture plate and allowed to adhere overnight. Medium was replaced with DMEM (Life Technologies) containing L-glutamine (Life Technologies). SiRNA-JetSi-ENDO complexes were made by first combining 0.2 μl JetSi-ENDO with 10 μl RPMI 1640 (Life Technologies) per well; this was incubated 20 minutes at room temperature to generate mix A. SiRNA duplexes were added to 10 μl RPMI-1640 to form mix B. Mix A was added to mix B and incubated at room temperature for 30 minutes. Complex AB (20 μl) was added drop-wise to the cells and incubated 4 hours. Transfection medium was then replaced with normal medium and cells were grown for 72 hours prior to assaying. BCE were first grown on the cytodex beads as described above, treated with siRNA duplexes and grown for 48 hours prior to being embedded in the collagen gel.

### 1.15 Transfection of Endothelial Cells

EVLC2 cells were transfected using Nucleofector technology (Amaxa, Cologne, Germany). Briefly, 5×10⁵ cells were harvested and resuspended in 100 μl Nucleofector solution R. The cell suspension was mixed with 1 μg DNA and transferred to an electroporation cuvette. Following transfection using program T20, 500 μl filtered HUVEC medium was added and cells were transferred to 2 wells of a gelatin coated 24-wells cell culture plate. Successfully transfected cells were selected based on hygromycin resistance using 25 μg/ml hygromycin (Life Technologies).

### 1.16 Chick Chorioallantoic Membrane (CAM) Assay

Fertilized white leghorn chicken eggs were used to monitor vessel development in the CAM as described previously (van der Schaft et al., 2000). Antibodies were dialysed to 0.9% NaCl and administered in the indicated concentrations in a volume of 65 μl for four consecutive days. On day 14, the CAMs were photographed. Five concentric rings were projected on the image. The number of intersections of rings and blood vessels was determined and used as a measure of vessel density. In some experiments, LS174T tumor tissues were placed on the chorioallantoic membrane (CAM) within the silicone ring. LS174T were seeded in non-adherent cell culture plates at a density of 10⁵ cells/ml for 10 days to allow spheroids to form. A small surface area (approximately 3×3 mm) in the silicone ring placed on the CAM was denudated using lens paper and uniformly looking spheroids of 0.5-1 mm in diameter were applied on the CAM. Antibodies were dialysed to 0.9% NaCl and administered in a volume of 65 μl for four consecutive days. At day 14, the CAMs were photographed.

### 1.17 Mouse Tumor Models

Female athymic nude mice were used and randomly split in four groups. All experiments were approved by the University of Minnesota Research Animal Resources ethical committee. Mice (n=6 per group) were inoculated with 1*10⁶ LS174T colorectal carcinoma cells in 100 RPMI subcutaneously in the right flank. Four days post-inoculation treatment was started. Mice were treated by i.p. injections every third day with a commercially available anti-TAG antibody, a commercially available isotype control antibody or saline. Tumor volume was determined daily by measuring the diameters of tumors using callipers and calculated as follows: width²×length×0.52.

Cryosections (5 μm) of the tumors were stained for CD31 and microvessel density was evaluated as described previously (Dings et al., 2003).

To assess the extent of total cell apoptosis, tissue sections are stained by using the TUNEL (terminal deoxyribo-nucleotidyl transferase-mediated dUTP-nick-end labelling) assay, which is performed according to the manufacturer's instructions (in situ cell death detection kit, fluorescein; TUNEL, Roche Applied Science).

### Example 2

### Identification of Tumor Endothelial Markers by SSH

A suppression subtractive hybridization (SSH) was performed in combination with cDNA array screening to identify novel tumor specific endothelial markers in an unbiased manner. Tumor endothelial cells (TEC) were successfully isolated from colon tumors (n=5) and patient-matched normal endothelial cells (NEC) from normal colon tissue samples (n=5), as well as from placenta tissues (PLEC, n=5) (FIG. 1). RNA was isolated (FIG. 1) and used to create subtraction repertoires of genes overexpressed in TEC. In addition, HUVEC were stimulated in vitro with tumor cell conditioned medium and used to create additional subtraction repertoires. A total of 2746 inserts, 1781 derived from the TEC subtractions and 965 derived from the HUVEC subtractions were amplified and spotted onto duplicate arrays that were probed with ³³P-dCTP labeled cDNA derived from TEC, NEC, PLEC and HUVEC. Phospho-imaging and pair-wise comparisons of spot intensities were performed to identify differentially expressed spots (FIG. 1). Insert identity was determined by sequencing analysis.

Transcripts showing overexpression in TEC vs NEC were further subdivided based on their expression in the other EC populations. By comparing expression profiles of TEC with NEC, PLEC, and HUVEC, it was possible to distinguish between genes associated with angiogenesis in general (general angiogenesis genes, GAGs) and genes specific for tumor endothelium (tumor angiogenesis genes, TAGs) (FIG. 2A, Table 1). Forty-one transcripts classified as TAGs (Table 1, FIGS. 2B & C, Table 1) and showed overexpression in both TEC compared to NEC and in TEC compared to PLEC. Eighty-five transcripts were found to be upregulated in TEC compared to NEC as well as in PLEC compared to NEC (GAG/A) (FIGS. 2B & D, Table 1). Finally, the 24 upregulated transcripts in activated HUVEC vs quiescent HUVEC as well as in TEC vs NEC are named GAG/B (FIGS. 2B & E, Table 1).

Sequence analysis revealed that the 41 TAG transcripts represented 17 different genes (Table 2). Five of these have previously been described to be overexpressed on tumor endothelium, validating our approach. The identification of the highly abundant collagens 4A1 and 1A1 in different tumor types points towards the possible existence of pan-tumor endothelium specific transcripts (Madden et al., 2004; Parker et al., 2004; St Croix et al., 2000). SPARC and IGFBP7 have also previously been associated with angiogenesis (Akaogi et al., 1996; Porter of al., 1995) and were classified as pan-endothelial markers (St Croix et al., 2000). HEYL, a basic helix-loop-helix transcription factor has recently been associated with breast tumor vasculature (Parker et al., 2004). PPAP2B has been described as a gene that is upregulated during in vitro tube formation of endothelial cells under the influence of VEGF (Humtsoe et al., 2003). Very recently, the cytokine HMGB1 was recognized for its role in promoting angiogenesis in vitro (Schlueter et al., 2005). The 10 remaining TAG markers have no reported functional contribution to tumors and/or angiogenesis.

### Example 3

### Gene Expression of Tumor Endothelial Cells is Closely Related to Gene Expression During Physiological Angiogenesis

It emerged that the majority of TEC overexpressed transcripts (85/142=60%) are also associated with angiogenesis under physiological conditions in vivo, and are therefore not specific for tumor angiogenesis in vivo (FIG. 2B). These 85 GAG/A transcripts represent 46 different genes, including genes that have been associated with angiogenesis such as matrix metalloproteinases (MMPs) (Pepper, 2001), integrin β1 (Senger et al., 2002) and endothelial cell specific molecule-1 (Aitkenhead et al., 2002) (Table 3).

### Example 4

### In Vitro Endothelial Cell Activation is a Limited Substitute for Studying Tumor Angiogenesis

From the gene expression analysis, it is obvious that only a limited number of genes upregulated in TEC vs NEC overlapped with genes overexpressed in tumor-conditioned HUVEC in vitro (GAG/B, FIG. 2B, Table 1). This suggested that this in vitro model may be of only limited value for studying tumor angiogenesis. Most of the GAG/B markers overlap with genes associated with angiogenesis in general (GAG/A) (FIG. 2B, Table 3). Hierarchical clustering analysis suggested that the expression pattern in the HUVEC model relates most to that emerging from physiological angiogenesis (i.e. the comparison between PLEC and NEC; FIG. 8).

### Example 5

### TAG Markers are Functionally Classified as Associated in Late Events of Angiogenesis

A functional annotation was assigned to every gene. The distribution of genes was analyzed into different functional classes. TAG markers are predominantly biased towards genes associated with cytoskeletal and extracellular matrix remodelling, indicative of late events in the process of tumor angiogenesis, whereas protein turnover and transcription associated genes are underrepresented within TAG (FIG. 2C). The GAG/A class represents genes associated to cell and protein turnover (FIG. 2D). Hallmarks of GAG/B molecules are active transcription and protein turnover (FIG. 2E). Functional clustering indicates that both GAG/A and GAG/B represent genes important in early events in the angiogenesis process.

Indeed, genes that showed overlapping expression profiles in activated HUVEC and tumor endothelium, GAG/B markers, were also highly biased towards protein turnover and transcription (Table 3). The fact that culture conditions highly influence gene expression was exemplified by the expression profile of HUVEC co-cultured with glioma cells (Khodarev et al., 2003). Encouragingly, genes related to ECM remodelling and cytoskeletal functions, suggestive of advanced stages of angiogenesis, were significantly upregulated in HUVEC co-cultured with tumor cells compared to monoculture of HUVEC. The direct physical contact of endothelial cells with the tumor cells seems necessary to direct this induction of gene expression.

### Example 6

### Validation of TAGs

Overexpression of TAGs was confirmed using real-time quantitative PCR(RTQ-PCR) as a second independent technique. For 16 different genes (94%) overexpression in TEC vs NEC was confirmed, also for 16 genes (94%) overexpression in TEC vs PLEC was confirmed (FIG. 3A). Taken together, 15 out of 17 (88%) genes validated by RTQ-PCR were positively confirmed TAG markers.

Of the 10 TAGs with no previous association with angiogenesis, 4 have no known function as yet.

For subsequent studies of TAG markers at the protein level, 4 different membrane associated or secreted molecules were selected: (i) CD59, a GPI membrane-anchored inhibitor of complement activation (Gelderman et al., 2004), (ii) insulin-like growth factor binding protein-7 (IGFBP7), a secreted molecule with growth factor modulating function (Akaogi et al., 1996), (iii) HMGB1, a secreted cytokine as well as a non-histone DNA binding protein (Goodwin et al., 1973; Treutiger et al., 2003), and (iv) vimentin, an intermediate filament protein that was recently demonstrated to be actively secreted (Mor-Vaknin et al., 2003; Xu et al., 2004). Immunohistochemical analysis in colorectal carcinoma and normal colon epithelium indicated that all 4 proteins were overexpressed on the tumor vasculature. While vimentin, IGFBP7 and CD59 were predominantly expressed in the endothelial compartment, HMGB1 was found to be expressed in stromal and epithelial cells as well (FIG. 3C). Vimentin expression was detected in endothelial cells of both tumor and normal colon tissue, though heavily overexpressed on tumor endothelium. IGFBP7 expression in normal colon tissue is hardly detected, whereas tumor blood vessels show abundant expression of IGFBP7. CD59 expression was mainly localized to vasculature, in particular to the luminal cell membrane (FIG. 3C). This is in line with its reported expression as membrane protein with a role in protecting endothelial cells from complement-mediated lysis by binding complement proteins C8 and C9 to prevent the formation of the membrane attack complex (Gelderman et al., 2004). HMGB1 staining was detected in endothelial cells, as cytoplasmic protein, but also in epithelial cells, where the localization was predominantly nuclear. In addition, diffuse stromal staining was observed. Protein expression was much more abundant in colorectal tumor tissue compared to normal colon tissue, predominantly in the stromal compartment, consistent with a secretion product (FIG. 3C) (Huttunen and Rauvala, 2004).

Since immunohistochemical analysis is a qualitative rather than a quantitative technique, the expression of our TAGs on freshly isolated endothelial cells of tumor and normal tissues was determined by flow cytometry. The overexpression of vimentin (TAG-39), IGFBP7 (TAG-29) and HMGB1 (TAG-21) protein on colon tumor endothelium compared to normal colon endothelium was quantitatively confirmed. In addition, the expression of CD31 did not differ between TEC and NEC (FIG. 3B). These observations further support the value of these proteins as tumor EC signature markers.

### Example 7

### Interference with TAG Proteins Inhibits Angiogenesis In Vitro and In Vivo

7.1 Interference with TAG Proteins Inhibits Angiogenesis in In Vitro Sprout-Formation Assay

To investigate whether the overexpression of the selected TAG markers is causally related to the process of angiogenesis, in vitro bioassays were performed. Antibodies directed against CD59, IGFBP7, vimentin and HMGB1 were tested for their effect on endothelial tube formation in an in vitro collagen-gel-based sprout-formation assay. Antibodies directed against the latter three showed inhibitory effects on BCE sprout formation in vitro, whereas CD59 and a control antibody were less effective (FIG. 4A). Capillary sprouting from isolated tumor vessels in an ex vivo set-up was inhibited by antibodies targeting HMGB1 and vimentin, and to a lesser extent CD59 (FIG. 4C).

These observations suggest that the targeted proteins are actively involved in the process of capillary tube formation.

7.2 Interference with TAG Proteins Inhibits Angiogenesis in In Vivo CAM Assay

To investigate whether these TAGs are involved in angiogenesis in vivo, the antibodies were tested in the developing chorioallantoic membrane (CAM) of the chick embryo. A similar result as in the sprouting assay was found for angiogenesis inhibition in the CAM in vivo (FIG. 4B). Antibodies against CD59, HMGB1 and vimentin inhibited angiogenesis by 27%, 45% and 40%, respectively, while a control antibody did not show any activity (as compared to CAMs treated with saline alone).

These results strongly suggest a role for these molecules in the process of angiogenesis and together with the overexpression on tumor endothelium support their potential for use in targeting of tumor vasculature as therapy against cancer.

### 7.3 Overexpression of TAG Proteins Promotes Angiogenesis in In Vivo Wound Assay

To further investigate the contribution of HMGB1 to tumor angiogenesis, we used expression constructs encoding HMGB1 in both the sense and the antisense orientation to induce or repress HMGB1 expression, respectively. Overexpression of HMGB1 clearly increased the migration speed of the endothelial cells in a wounding assay (FIG. 5A). Also, MMP9 expression was induced in HMGB1 sense expressing cells. In addition, response to growth factor activation was impaired in HMGB1 antisense expressing cells (FIG. 5C).

### 7.4 Downregulation of TAG Proteins by RNA Interference Inhibits Angiogenesis

RNA interference was employed to investigate the effect of downregulated TAG levels in endothelial cells. Different concentrations siRNA duplex specific for vimentin were capable of inhibiting migration of the cells (FIG. 5D), as well as sprouting (FIG. 5E). Only at higher concentrations of siRNA duplex, the proliferation of the cells is impaired, suggesting toxicity is non-existing at 50 nM siRNA duplex (FIG. 5E). Similar results are obtained by downregulation of HMGB1 by different concentrations siRNA duplex specific for HMGB1.

### 7.5 Use of TAGs

Although HMGB1/amphoterin was originally identified as a non-histone DNA binding molecule (Goodwin et al., 1973), more recently focus has shifted to its role as a secreted cytokine. As an extracellular protein, it has been involved in the regulation of cell migration (Fages et al., 2000), tumorigenesis (Taguchi et al., 2000), cell activation (Treutiger et al., 2003) and inflammation (Fiuza et al., 2003). It can act as a paracrine or autocrine factor creating feedback loops for the secretion of TNF-α and IL-1β in monocytes and macrophages. It also acts on endothelial cells to upregulate ICAM-1, VCAM-1 and TNF-α expression (Fiuza et al., 2003; Treutiger et al., 2003) and stimulates sprouting (Schlueter et al., 2005). We have demonstrated that an antibody directed at HMGB1 was effective at inhibiting endothelial cell sprouting in vitro as well as angiogenesis in vivo. This finding can be exploited for therapeutic modulation of angiogenesis, e.g. inhibiting tumor angiogenesis as shown in FIG. 4, or stimulation of angiogenesis in ischemic diseases.

Vimentin is an extensively studied intermediate filament protein (reviewed by Hendrix et al., 1996) which has also been described as a target gene of HIF-1α, a major inducer of VEGF (Krishnamachary et al., 2003). We have shown quantitative data on the overexpression of vimentin on endothelial cells in colon tumor samples compared to normal colon samples, both at the transcriptional level (FIG. 3A) and the protein level (FIG. 3B). This result suggests a contribution of this protein to the tumor endothelial phenotype. We present evidence that targeting of vimentin by means of antibodies clearly inhibited angiogenesis both in vitro and in vivo.

CD59 is a GPI anchored membrane protein and an inhibitor of complement activation (Gelderman et al., 2004). CD59 function is dependent upon complement activation.

Complement activation does not apply in vitro, which explains our result that antibodies directed against CD59 were not readily effective in our in vitro assays. Targeting of CD59 in vivo is more successful. Indeed, in vivo in the CAM assay, a significant effect of anti-CD59 antibodies on vessel formation was demonstrated.

IGFBP7 is a secreted protein that accumulates in the basement membrane (Ahmed et al., 2003; Akaogi et al., 1996), where it can bind collagens type 2, 4 and 5, heparan sulfates and different cytokines (Akaogi et al., 1996; Nagakubo et al., 2003). By binding collagens it supports the organization of endothelial cells into tube-like structures (Akaogi et al., 1996). In summary, it is known that IGFBP7 functions in blood vessels. The present study demonstrated that overexpression of IGFBP7 in tumor endothelium was evident both at the transcriptional level and at the protein level. In addition, targeting IGFBP7 with an antibody clearly inhibited endothelial sprouting in vitro, possibly caused by inhibition of the interaction between IGFBP7 and collagens present in the three-dimensional culture matrix.

From the series of 17 TAGs, several different genes encoded membrane-bound or secreted proteins. Four of these were selected to investigate for a role in angiogenesis and to serve as tumor endothelial target for therapeutic applications. The present invention demonstrated that all four genes (i) are necessary in the process of angiogenesis and (ii) can be used for intervention in angiogenesis using antibodies as a treatment opportunity.

### Example 8

### Interference with TAG Proteins Inhibits Tumor Associated Angiogenesis

To further investigate the relation between the selected TAG markers and the process of angiogenesis, the following experiments were conducted.

### 8.1 Tumor Angiogenesis is Inhibited in CAM Assay

An experimental model of tumor angiogenesis was set up that employs the growth of LS174T colon carcinoma cell spheroids transplanted onto the CAM. Growth of these spheroids induces the growth of vasculature and induced aberrant morphology in the chick vasculature (FIG. 6). Treatment of the CAMs with commercially available antibodies against HMGB1 (FIG. 6A) and vimentin (FIG. 6B) shows a reduction in vessel density on the CAM as compared to untreated tumors.

To provide proof of principle that targeting of TAGs inhibits tumor angiogenesis and tumor growth, antibodies against CD59, HMGB1 and vimentin were tested in the model of a transplanted tumor onto the CAM. In this model a lump of 1 mm³ LS174T human colon tumor tissue is put on a 10-day CAM. Antibody treatment, performed as described above, resulted in significant inhibition of vessel growth and in reduction of tumor growth (FIG. 7).

### 8.2 Tumor Angiogenesis is Inhibited in Nude Mice Assay

To provide an absolute proof of the feasibility of the invention to identify endothelial targets for cancer treatment, LS174T colon carcinoma model in nude mice is used. The nude mice are treated with the mouse-reactive anti-HMGB-1 antibody (clone HAP46.5). Treatment of tumor-bearing mice with HAP46.5 shows a dose-dependent inhibition of tumor growth with no apparent toxic effects. Microvessel density of the HAP46.5 treated tumors is markedly reduced, whereas tumors treated with the control antibody show no inhibition of tumor growth or inhibition of microvessel density. Furthermore, there is no apparent toxicity associated with the treatment as the body weight of the mice does not differ between the treatment groups (FIG. 7D), suggesting no or only limited effects of the TAG antibody on normal body physiology.

The above proof of the feasibility of the invention to identify endothelial targets for cancer treatment was corroborated by experiments with anti-vimentin antibodies. In particular, nude mice bearing LS174T tumors are treated with the mouse-reactive anti-vimentin antibody. It is verified that the tumor cells do not express vimentin (FIG. 7B, panels a and b) to ascertain that effects of the treatment would be the result of targeting the vasculature.

Treatment of tumor-bearing mice with antibody shows a dose-dependent inhibition of tumor growth with no apparent toxic effects. Microvessel density of the antibody treated tumors is markedly reduced, whereas tumors treated with the control antibody shows no inhibition of tumor growth or inhibition of microvessel density. Furthermore, there is no apparent toxicity associated with the treatment as the body weight of the mice did not differ between the treatment groups, suggesting no or only limited effects of the vimentin antibody on normal body physiology (Van Beijnum et al. Blood, in press, June 2006, incorporated herein explicitly by reference).

Further substantiation of the feasibility of the invention identifying endothelial targets for cancer treatment comes from the LS174T colon carcinoma model in nude mice as follows.

The nude mice are treated with commercially available antibodies against ARPC2, CDK2AP1, Col1A1, HEYL, LDHB, PPAP2B, SPARC. Treatment of tumor-bearing mice with these antibodies shows a dose-dependent inhibition of tumor growth with no apparent toxic effects. Microvessel density of the treated tumors is markedly reduced, whereas tumors treated with the control antibody show no inhibition of tumor growth or inhibition of microvessel density. Furthermore, there is no apparent toxicity associated with the treatment as the body weight of the mice does not differ between the treatment groups, suggesting no effects of the antibodies on normal body physiology.

For Col4A1, TAG-23, TAG-27, HSIRPR, PHC3 and HSPC014 antibodies are produced and tested similarly in mouse tumor models as described above for the other TAGs.

### Example 9

### Targeting TAG Proteins does not Cause Side Effects Associated with Impaired Physiological Angiogenesis

TAG proteins are selected on their differential expression pattern in endothelial cells, i.e. higher expression in angiogenic tumor endothelial cells compared to normal resting or normal angiogenic endothelial cells. Therefore, targeting TAGs as a means of therapeutic inhibition of endothelial cells results in an expression-dependent inhibition of angiogenesis. Targeting is most effective in endothelial cells with the highest expression of the TAG. This creates a certain degree of specificity of tumor directed anti-angiogenic therapy and reduces side effects associated with inhibition of physiological angiogenesis.

To determine the magnitude of effects of targeting TAG proteins on physiological angiogenesis, wound healing in mice is studied. Circular wounds of 6 mm diameter are made through the skin at the back of the mouse, according to the method described by Eckes et al. (Eckes et al., J Cell Sci 113, 2000). Mice are treated with vehicle alone (Control group A), treated with different concentrations of a TAG-specific antibody (Experimental group B), and treated with a non-relevant antibody (Control group C). The size of the wound is measured daily and closure of the wound is a representative measure of physiological angiogenesis. No significant differences in wound healing are observed between the different treatment groups.

It is concluded that treatment of tumors using TAG-specific antibodies causes no side effects associated with impaired physiological angiogenesis.

TABLE 1
Characteristics of EC gene expression identified by differential
screening of SSH repertoires

| Classification of tumor endothelial markers^{d} | | Spots^{e} | Gene IDs^{f} |
|---|---|---|---|
| TAG^{a} | TEC > NEC and TEC > PLEC | 41 | 17 |
| GAG/A^{b} | TEC > NEC and PLEC > NEC | 85 | 46 |
| GAG/B^{c} | TEC > NEC and HUVEC+ > HUVEC− | 24 | 22 |

^{a}Tumor specific EC markers
^{b}Common angiogenesis markers
^{c}In vitro and in vivo markers
^{d}TEC: Tumor endothelial cell; NEC: Normal endothelial cell; PLEC: Placenta endothelial cell; HUVEC+: tumor-conditioned HUVEC; HUVEC−: quiescent HUVEC
^{e}Number of spots that showed at least 2-fold difference in expression in the indicated comparisons
^{f}Number of different genes representing differentially expressed spots

TABLE 2
Tumor angiogenesis genes (TAGs)

| | | | Accession | | | No of | Library origin^{f} | |
|---|---|---|---|---|---|---|---|---|
| TAG^{a} | Gene ID^{b} | | No^{c} | SEQ ID NO: s | Function^{d} | spots^{e} | HUVEC | TEC |
| TAG-1 | Actin related protein 2/3 complex | ARPC2 | NM_152862.1 | 1, 2, 35, 36 | Cytoskeleton | 2 | | x |
| TAG-3 | CD59 antigen p18-20 | CD59 | NM_000611 | 3, 4, 27 | Cell surface | 1 | | x |
| TAG-4 | CDK2-associated protein 1 | CDK2AP1 | NM_004642.2 | 5, 6, 38 | Apoptosis, Cell cycle | 1 | | x |
| TAG-5 | Collagen 1A1 | Col1A1 | NM_000088.2 | 7, 8, 39, 40, 41, 42 | Extracellular matrix | 4 | | x |
| TAG-7 | Collagen 4A1 | Col4A1 | NM_001845.2 | 9, 10, 43-56 | Extracellular matrix | 16 | x | x |
| TAG- | High mobility group protein-1 | HMGB1 | NM_002128.3 | 11, 12, 57 | Extracellular matrix | 1 | | x |
| 21 | | | | | | | | |
| TAG- | IMAGE 5299642 | EST | BC041913 | 13, —, 58 | Unknown | 1 | x | |
| 23 | | | | | | | | |
| TAG- | Hairy/enhancer of split with YRPW | HEYL | NM_014571 | 15, 16, 59 | Transcription | 1 | | x |
| 25 | motif | | | | | | | |
| TAG- | IMAGE 4332094 | EST | NM_017994.1 | 17, 18, 60 | Unknown | 1 | | x |
| 27 | | | | | | | | |
| TAG- | Insulin receptor precursor | HSIRPR | X02160 | 19, 20, 61 | Cell surface | 1 | | x |
| 28 | | | | | | | | |
| TAG- | Insulin-like growth factor binding | IGFBP7 | NM_001553 | 21, 22, 62 | Extracellular matrix | 1 | x | |
| 29 | protein 7 | | | | | | | |
| TAG- | Lactate dehydrogenase B | LDHB | NM_002300 | 23, 24, 63 | Metabolism, cell | 1 | x | |
| 30 | | | | | maintenance | | | |
| TAG- | Phosphatidic acid phosphatase type | PPAP2B | CV337080 | 25, —, 64 | Metabolism, cell | 1 | | x |
| 31 | 2B | | | | maintenance | | | |
| TAG- | Polyhomeotic like 3 | PHC3 | AJ320486 | 27, 28, 65 | Unknown | 1 | | x |
| 32 | | | | | | | | |
| TAG- | Secreted protein acidic, rich in | SPARC | NM_003118.1 | 29, 30, 66-70 | Extracellular matrix | 5 | | x |
| 33 | cysteine | | | | | | | |
| TAG- | Voltage gated K channel beta subunit | HSPC014 | AF077200 | 31, 32, 71 | Unknown | 1 | x | |
| 38 | 4.1 | | | | | | | |
| TAG- | Vimentin | VIM | X56134 | 33, 34, 72-74 | Cytoskeleton | 3 | x | x |
| 39 | | | | | | | | |

^{a}TAG: Tumor angiogenesis gene
^{b}Sequence identity
^{c}GenBank accession number
^{d}Functional classification of the reported TAG
^{e}Number of spots that represented the TAG
^{f}SSH repertoire origin of the spots

TABLE 3
General angiogenesis genes (GAG/A and GAG/B)

| | | Accession | | | No | Library origin^{f} | |
|---|---|---|---|---|---|---|---|
| Gene ID^{a} | | No^{b} | Class^{c} | Function^{d} | of spots^{e} | HUVEC | TEC |
| A kinase (PRKA) anchor protein 13 (AKAP13) | AKAP13 | NM_007200 | A | Signaling | 1 | x | |
| ATP synthase H+ transporting complex, subunit c | ATP5G1 | NM_001575 | A, B | Metabolism, cell | 1 | x | |
| | | | | maintenance | | | |
| Cathepsin B | CTSB | NM_147783.1 | A | Extracellular matrix | 12 | | x |
| HSP90 alpha | HSPCA | NM_005348 | A, B | Metabolism, cell | 1 | x | |
| | | | | maintenance | | | |
| Caveolin 1, caveolae protein, 22 kDa | CAV1 | NM_001753 | A | Receptor | 2 | x | |
| IMAGE 757234 | EST | BX115183 | A, B | Unknown | 1 | x | |
| dCMP deaminase | DCTD | NM_001921.1 | A | Metabolism, cell | 1 | | x |
| | | | | maintenance | | | |
| Defender against cell death 1 | DAD1 | NM_001344 | A | Apoptosis, Cell cycle | 1 | x | |
| Split hand/foot malformation (ectrodactyly) type 1 | SHFM1 | NM_006304 | A, B | Apoptosis, Cell cycle | 1 | x | |
| Ectonucleoside triphosphate diphosphohydrolase 1 | ENTPD1 | BC047664 | A, B | Receptor | 1 | | x |
| Endothelial cell-specific molecule 1 | ESM1 | NM_007036 | A, B | Receptor | 1 | x | |
| IMAGE 2816112 | EST | AW269823 | A | Unknown | 1 | x | |
| SRY (sex-determining region Y)-box 4 | SOX4 | NM_003107 | A, B | Transcription | 2 | x | |
| Eukaryotic translation elongation factor 1 epsilon 1 | EEF1E1 | NM_004280.2 | A, B | Protein turnover | 2 | x | |
| FK506 binding protein 1A, 12 kDa | FKBP1A | NM_000801 | A | Signaling | 1 | x | |
| cDNA FLJ32199 clone PLACE6002710 | EST | AK056761 | A | Unknown | 1 | x | |
| Heterogeneous nuclear ribonucleoprotein C C1/C2) | HNRPC | BC003394 | A, B | Protein turnover | 1 | x | |
| Hypothetical protein MGC 7036 | MGC7036 | NM_145058 | A | Unknown | 1 | | x |
| Integrin beta 1 | ITGB1 | NM_002211 | A | Receptor | 1 | x | |
| Rad51 associated protein | RAD51AP1 | NM_006479 | A | Metabolism, cell | 1 | x | |
| | | | | maintenance | | | |
| Isoprenylcysteine carboxyl methyltransferase | ICMT | NM_170705.1 | A | Metabolism, cell | 1 | | x |
| | | | | maintenance | | | |
| Mitochondrial ribosomal protein S27 | MRPS27 | BC011818 | A, B | Protein turnover | 1 | x | |
| F-box protein 9 | FBXO9 | NM_033480 | A, B | Protein turnover | 1 | | x |
| Zinc finger motif enhancer binding protein 2 | ZNF644 | NM_032186 | A, B | Transcription | 1 | x | |
| Matrix metalloproteinase 1, interstitial collagenase | MMP1 | NM_002421 | A | Extracellular matrix | 1 | x | |
| Matrix metalloproteinase 10, stromelysin 2 | MMP10 | NM_002425 | A, B | Extracellular matrix | 2 | x | |
| Eukaryotic translation initiation factor 4A, isoform 1 | EIF4A1 | NM_001416 | A | Protein turnover | 1 | x | |
| Androgen-induced 1 | AIG-1 | BC025278 | A, B | Unknown | 1 | x | |
| Caspase recruitment domain family, member 8 | CARD8 | NM_014959 | A, B | Apoptosis, Cell cycle | 1 | x | |
| Diazepam binding inhibitor | DBI | M15887 | A, B | Signaling | 1 | x | |
| IMAGE 2028956 | EST | AI793182 | A | Unknown | 1 | x | |
| Major histocompatibility complex, class II, DR alpha | HLA-DRA | BC032350 | A | Surface antigen | 23 | | x |
| Chemokine (C-C motif) ligand 2 | CCL2 | NM_002982 | A | Signaling | 1 | x | |
| Matrix metalloproteinase 3, stromelysin 1 | MMP3 | NM_002422 | A | Extracellular matrix | 1 | | x |
| Thymosin, beta 4, X-linked | TMSB4X | NM_021109 | A, B | Cytoskeleton | 1 | x | |
| v-ral simian leukemia viral oncogene homolog B | RALB | BC018163 | A | Signaling | 1 | | x |
| EST10870 HUVEC | EST | AA296386 | A | Unknown | 2 | x | |
| Ubiquitin conjugating enzyme E2L3, | UBE2L3 | NM_003347 | A, B | Protein turnover | 1 | x | |
| IMAGE 2096486 | EST | AI422919 | A | Unknown | 1 | | x |
| Pituitary tumor-transforming 1 interacting protein | PTTG1IP | NM_004339.2 | A | Signaling | 1 | | x |
| Tubulin, alpha 3 | TUBA3 | BC050637 | A | Cytoskeleton | 1 | x | |
| Lung cancer oncogene 5 | HLC5 | AY117690.1 | A | Unknown | 1 | x | |
| Heme binding protein 1 | HEBP1 | NM_015987.2 | A | Metabolism, cell | 1 | | x |
| | | | | maintenance | | | |
| FLJ37490 | EST | AK094809.1 | A | Unknown | 2 | | x |
| Ribosomal protein L22 | RPL22 | NM_000983 | A | Protein turnover | 1 | x | |
| CD86 antigen | CD86 | NM_175862 | A | Surface antigen | 1 | | x |
| Platelet/endothelial cell adhesion molecule 1 | PECAM1 | NM_000442.2 | B | Surface antigen | 1 | x | |
| Ribosomal protein L21 | RPL21 | NM_000982.2 | B | Protein turnover | 2 | x | |

^{a}Sequence identity
^{b}GenBank accession number
^{c}GAG class
^{d}Functional classification of the reported GAG
^{e}Number of spots that represented the GAG
^{f}SSH repertoire origin of the spots

TABLE 4
Amplification primers for TAGs

| | | | | SEQ | | SEQ | | | |
|---|---|---|---|---|---|---|---|---|---|
| | | Accession | | ID | | ID | | | |
| TAG | Gene ID | No | Forward 5′-3′ | NO: | Reverse 5′-3′ | NO: | Start | Stop | Length |
| TAG-1 | ARPC2 | NM_152862.1 | GCAACTGAAGGCTGGAACA | 75 | TGAAGAGGCGCAACATTAAA | 76 | 1044 | 1105 | 62 |
| TAG-3 | CD59 | NM_203330 | AGTGGGTGAATGTGGTTAT | 77 | CTGGCACTGCTCAGGATGTC | 78 | 1739 | 1823 | 85 |
| | | | GGCC | | TTC | | | | |
| TAG-4 | CDK2AP1 | NM_004642.2 | GCTGGCCATCATTGAAGAGCT | 79 | TCTCCATGGCACTCTTGCT | 80 | 720 | 790 | 71 |
| | | | | | CC | | | | |
| TAG-5 | Col1A1 | NM_000088.2 | GAGATCGAGATCCGCGC | 81 | TGCAGCCATCGACAGTGAC | 82 | 4302 | 4365 | 64 |
| TAG-7 | Col4A1 | NM_001845.2 | GGCACCCCATCTGTTGATCAC | 83 | GGTAAAGAATTTTGGTCCC | 84 | 4441 | 4534 | 94 |
| | | | | | AGAA | | | | |
| TAG-21 | HMGB1 | NM_002128 | TCTAAGAAGTGCTCAGAG | 85 | TTCATTTCTCTTTCATAAC | 86 | 219 | 322 | 104 |
| | | | AGGTG | | GGG | | | | |
| TAG-23 | EST | BC041913 | CAAATTCACTAGGCAAGCGGA | 87 | GGTTGTCCCTTTAATGCAG | 88 | 348 | 452 | 105 |
| | | | | | CTT | | | | |
| TAG-25 | HEYL | NM_014571 | TCTGAGCTGCCCCTTCACCAC | 89 | ACGTGCCTTCACATATGA | 90 | 1004 | 1125 | 122 |
| | | | | | GCCAG | | | | |
| TAG-27 | EST | NM_017994 | ATTTTGTCCCGAGAAGGTGGC | 91 | AGCAGGCAAGGATTATGG | 92 | 1142 | 1234 | 93 |
| | | | | | TTCTC | | | | |
| TAG-28 | HSIRPR | X02160 | TTCTCAAGGGTGCGAGCTC | 93 | TCCTCCCTTGGCCACCAATG | 94 | 4885 | 5022 | 138 |
| | | | ATC | | | | | | |
| TAG-29 | IGFBP7 | NM_001553 | AAGGGGTCACTATGGAG | 95 | GGCACTCATATTCTCCAG | 96 | 613 | 766 | 153 |
| | | | TTCAAA | | CATCT | | | | |
| TAG-30 | LDHB | NM_002300 | TGGGCTATTGGATTAAGT | 97 | TTGACACGGGATGAATCCT | 98 | 853 | 928 | 76 |
| | | | GTGGC | | GG | | | | |
| TAG-31 | PPAP2B | CV337080 | TGGGGAGAATCACATTTG | 99 | ATGGCTTCAGAGCTGGTC | 100 | 130 | 240 | 111 |
| | | | GGTC | | ATGG | | | | |
| TAG-32 | PHC3 | AJ320486 | TCGCAGATGAATTCAGAG | 101 | TTGATGCGTGCACAGATC | 102 | 2874 | 2997 | 123 |
| | | | CACAG | | TTCAG | | | | |
| TAG-33 | SPARC | NM_003118 | GCACCACCCGCTTTTTC | 103 | GATCCTTGTCGATATCCT | 104 | 851 | 958 | 108 |
| | | | | | TCTG | | | | |
| TAG-38 | HSPC014 | AF077200 | CATTCAGGGTCTATTTGC | 105 | GAAGACGCTGAACCTGCTGC | 106 | 289 | 359 | 71 |
| | | | TCCG | | | | | | |
| TAG-39 | VIM | X56134 | ACACACTCAGTGCAGCAAT | 107 | GGAGTGTCGGTTGTTAAGA | 108 | 913 | 1064 | 152 |
| | | | ATAT | | ACTA | | | | |

### REFERENCES

Abe, M., and Sato, Y. (2001). cDNA microarray analysis of the gene expression profile of VEGF-activated human umbilical vein endothelial cells. Angiogenesis 4, 289-298.
Ahmed, S., Yamamoto, K., Sato, Y., Ogawa, T., Herrmann, A., Higashi, S., and Miyazaki, K. (2003). Proteolytic processing of IGFBP-related protein-1 (TAF/angiomodulin/mac25) modulates its biological activity. Biochem Biophys Res Commun 310, 612-618.
Aitkenhead, M., Wang, S. J., Nakatsu, M. N., Mestas, J., Heard, C., and Hughes, C. C. (2002). Identification of endothelial cell genes expressed in an in vitro model of angiogenesis: induction of ESM-1, (beta)ig-h3, and NrCAM. Microvasc Res 63, 159-171.
Akaogi, K., Okabe, Y., Sato, J., Nagashima, Y., Yasumitsu, H., Sugahara, K., and Miyazaki, K. (1996). Specific accumulation of tumor-derived adhesion factor in tumor blood vessels and in capillary tube-like structures of cultured vascular endothelial cells. Proc Natl Acad Sci USA 93, 8384-8389.
Altschul et al. (1997) Gapped BLAST and PSI-BLAST: a new generation of protein database search programs. Nucleic Acids Res. 1997 Sep. 1; 25(17):3389-402.
de Hoon, M. J., Imoto, S., Nolan, J., and Miyano, S. (2004). Open source clustering software. Bioinformatics 20, 1453-1454.
Dell'Era, P., Coco, L., Ronca, R., Sennino, B., and Presta, M. (2002). Gene expression profile in fibroblast growth factor 2-transformed endothelial cells. Oncogene 21, 2433-2440.
Dings et al. (2003) β-Sheet is the bioactive conformation of the anti-angiogenic anginex peptide. Biochem. J. 373: 281-288.
Fages, C., Nolo, R., Huttunen, H. J., Eskelinen, E., and Rauvala, H. (2000). Regulation of cell migration by amphoterin. J Cell Sci 113 (Pt 4), 611-620.
Fernandes and Gregoriadis, 1997 Polysialylated asparaginase: preparation, activity and pharmacokinetics. Biochim Biophys Acta. 1997 Aug. 15; 1341(1):26-34.
Fiuza, C., Bustin, M., Talwar, S., Tropea, M., Gerstenberger, E., Shelhamer, J. H., and Suffredini, A. F. (2003). Inflammation-promoting activity of HMGB1 on human microvascular endothelial cells. Blood 101, 2652-2660.
Gelderman, K. A., Tomlinson, S., Ross, G. D., and Goiter, A. (2004). Complement function in mAb-mediated cancer immunotherapy. Trends Immunol 25, 158-164.
Gerritsen, M. E., Tomlinson, J. E., Zlot, C., Ziman, M., and Hwang, S. (2003b). Using gene expression profiling to identify the molecular basis of the synergistic actions of hepatocyte growth factor and vascular endothelial growth factor in human endothelial cells. Br J Pharmacol 140, 595-610.
Goodwin, G. H., Sanders, C., and Johns, E. W. (1973). A new group of chromatin-associated proteins with a high content of acidic and basic amino acids. Eur J Biochem 38, 14-19.
Hendrix, M. J., Seftor, E. A., Chu, Y. W., Trevor, K. T., and Seftor, R. E. (1996). Role of intermediate filaments in migration, invasion and metastasis. Cancer Metastasis Rev 15, 507-525.
Humtsoe, J. O., Feng, S., Thakker, G. D., Yang, J., Hong, J., and Wary, K. K. (2003). Regulation of cell-cell interactions by phosphatidic acid phosphatase 2b/VCIP. Embo J 22, 1539-1554.
Huttunen, H. J., and Rauvala, H. (2004). Amphoterin as an extracellular regulator of cell motility: from discovery to disease. J Intern Med 255, 351-366.
Jayasena (1999) Aptamers: an emerging class of molecules that rival antibodies in diagnostics. Clin Chem. 1999 September; 45(9):1628-50.
Khodarev, N. N., Yu, J., Labay, E., Darga, T., Brown, C. K., Mauceri, H. J., Yassari, R., Gupta, N., and Weichselbaum, R. R. (2003). Tumor-endothelium interactions in co-culture: coordinated changes of gene expression profiles and phenotypic properties of endothelial cells. J Cell Sci 116, 1013-1022.
Krishnamachary, B., Berg-Dixon, S., Kelly, B., Agani, F., Feldser, D., Ferreira, G., Iyer, N., LaRusch, J., Pak, B., Taghavi, P., and Semenza, G. L. (2003). Regulation of colon carcinoma cell invasion by hypoxia-inducible factor 1. Cancer Res 63, 1138-1143.
Madden, S. L., Cook, B. P., Nacht, M., Weber, W. D., Callahan, M. R., Jiang, Y., Dufault, M. R., Zhang, X., Zhang, W., Walter-Yohrling, J., et al. (2004). Vascular gene expression in nonneoplastic and malignant brain. Am J Pathol 165, 601-608.
Mor-Vaknin, N., Punturieri, A., Sitwala, K., and Markovitz, D. M. (2003). Vimentin is secreted by activated macrophages. Nat Cell Biol 5, 59-63.
Nagakubo, D., Murai, T., Tanaka, T., Usui, T., Matsumoto, M., Sekiguchi, K., and Miyasaka, M. (2003). A high endothelial venule secretory protein, mac25/angiomodulin, interacts with multiple high endothelial venule-associated molecules including chemokines. J Immunol 171, 553-561.
Parker, B. S., Argani, P., Cook, B. P., Liangfeng, H., Chartrand, S. D., Zhang, M., Saha, S., Bardelli, A., Jiang, Y., St Martin, T. B., et al. (2004). Alterations in vascular gene expression in invasive breast carcinoma. Cancer Res 64, 7857-7866.
Pepper, M. S. (2001). Role of the matrix metalloproteinase and plasminogen activator-plasmin systems in angiogenesis. Arterioscler Thromb Vasc Biol 21, 1104-1117.
Porter, P. L., Sage, E. H., Lane, T. F., Funk, S. E., and Gown, A. M. (1995). Distribution of SPARC in normal and neoplastic human tissue. J Histochem Cytochem 43, 791-800.
Schlueter, C., Weber, H., Meyer, B., Rogalla, P., Roser, K., Hauke, S., and Bullerdiek, J. (2005). Angiogenetic signaling through hypoxia: HMGB1: an angiogenetic switch molecule. Am J Pathol 166, 1259-1263.
Senger, D. R., Perruzzi, C. A., Streit, M., Koteliansky, V. E., de Fougerolles, A. R., and Detmar, M. (2002). The alpha(1)beta(1) and alpha(2)beta(1) integrins provide critical support for vascular endothelial growth factor signaling, endothelial cell migration, and tumor angiogenesis. Am J Pathol 160, 195-204.
St Croix, B., Rago, C., Velculescu, V., Traverso, G., Romans, K. E., Montgomery, E., Lal, A., Riggins, G. J., Lengauer, C., Vogeistein, B., and Kinzler, K. W. (2000). Genes expressed in human tumor endothelium. Science 289, 1197-1202.
Taguchi, A., Blood, D. C., del Toro, G., Canet, A., Lee, D. C., Qu, W., Tanji, N., Lu, Y., Lalla, E., Fu, C., et al. (2000). Blockade of RAGE-amphoterin signalling suppresses tumor growth and metastases. Nature 405, 354-360.
Treutiger, C. J., Mullins, G. E., Johansson, A. S., Rouhiainen, A., Rauvala, H. M., Erlandsson-Harris, H., Andersson, U., Yang, H., Tracey, K. J., Andersson, J., and Palmblad, J. E. (2003). High mobility group 1 B-box mediates activation of human endothelium. J Intern Med 254, 375-385.
Van Beijnum, J. R., and Griffioen, A. W. (2005). In silico analysis of angiogenesis associated gene expression identifies angiogenic stage related profiles. Biochim Biophys Acta In press.
van der Schaft, D. W., Toebes, E. A., Haseman, J. R., Mayo, K. H., and Griffioen, A. W. (2000). Bactericidal/permeability-increasing protein (BPI) inhibits angiogenesis via induction of apoptosis in vascular endothelial cells. Blood 96, 176-181.
van Leeuwen E B, Wisman G B, Tervaert J W, Palmans L L, van Wijk R T, Veenstra R, Molema G, van der Zee A G, van der M J, Ruiters M H. An SV40 large T-antigen immortalized human umbilical vein endothelial cell line for anti-endothelial cell antibody detection. Clin Exp Rheumatol. 2001; 19: 283-290
Wang, B., Xiao, Y., Ding, B. B., Zhang, N., Yuan, X., Gui, L., Qian, K. X., Duan, S., Chen, Z., Rao, Y., and Geng, J. G. (2003). Induction of tumor angiogenesis by Slit-Robo signaling and inhibition of cancer growth by blocking Robo activity. Cancer Cell 4, 19-29.
Xu, B., DeWaal, R. M., Mor-Vaknin, N., Hibbard, C., Markovitz, D. M., and Kahn, M. L. (2004). The Endothelial Cell-Specific Antibody PAL-E Identifies a Secreted Form of Vimentin in the Blood Vasculature. Mol Cell Biol 24, 9198-9206.
Zhang, H.-T., Gorn, M., Smith, K., Graham, A. P., Lau, K. K. W., and Bicknell, R. (1999). Transcriptional profiling of human microvascular endothelial cells in the proliferative and quiescent state using cDNA arrays. Angiogenesis 3, 211-219.

What is claimed is:

1. A method of diagnosing a pathological condition or a susceptibility to a pathological condition, said method comprising:
(a) contacting a tissue sample with an antibody specific for the protein encoded by SEQ ID NO:18 or a part thereof or with a probe specific for SEQ ID NO: 17 or a portion thereof;
(b) detecting binding of said antibody or probe to said tissue sample;
(c) comparing the binding detected in step (b) with a standard,
wherein a difference in binding relative to the standard is diagnostic of a pathological condition or a susceptibility to a pathological condition.

2. The method according to claim 1, wherein said detecting is performed by FACS.

3. A method of reducing the risk of, treating and/or alleviating proliferative disorders, or for stimulating or inhibiting angiogenesis in a patient in need thereof comprising administering the protein represented by SEQ ID NO: 18 or portion thereof or an antibody to the protein or portion thereof wherein the nucleic acid corresponding to the protein represented by SEQ ID NO: 18 is selected from the group consisting of:
(a) a nucleic acid comprising the DNA sequence as represented by SEQ ID NO: 17 or a part thereof, or the complement thereof,
(b) a nucleic acid comprising the RNA sequence corresponding to SEQ ID NO: 17 or a part thereof, or the complement thereof,
(c) a nucleic acid specifically hybridizing to the nucleotide sequence as defined in (a) or (b),
(d) a nucleic acid which is at least 65% identical to the sequence defined in (a),
(e) a nucleic acid encoding a protein with an amino acid sequence, which is at least 65% identical to the amino acid sequence as represented by SEQ ID NO: 18 or a part thereof,
(f) a nucleic acid encoding a protein comprising the amino acid sequence as represented by SEQ ID NO: 18 or a part thereof,
(g) a nucleic acid which is degenerated as a result of the genetic code to a nucleotide sequence of a nucleic acid as represented by SEQ ID NO: 17 or a part thereof or as defined in (a) to (f),
(h) a nucleic acid which is diverging due to the differences in codon usage between the organisms to a nucleotide sequence encoding a protein as represented by SEQ ID NO: 18 or as defined in (a) to (g),
(i) a nucleic acid which is diverging due to the differences between alleles encoding the protein as represented by SEQ ID NO: 18 or as defined in (a) to (h),
(j) a nucleic acid encoding an immunologically active and/or functional fragment of the protein encoded by the DNA sequence as represented by SEQ ID NO: 17,
(k) a nucleic acid encoding a gene family member of the nucleic acid as represented by SEQ ID NO: 17 and,
(l) a nucleic acid encoding the protein as represented by SEQ ID NO: 18 or a nucleic acid as defined in any one of (a) to (k) characterized in that said sequence is DNA, cDNA, genomic DNA or synthetic DNA;
wherein said isolated polypeptide is encodable by a nucleic acid according to (a) to (1), or a variant or a derivative thereof, or an immunologically active and/or functional fragment thereof; and
wherein said antibody specifically recognizes said polypeptide or a specific epitope of said polypeptide.

4. A method for diagnosing a pathological condition or a susceptibility to a pathological condition, comprising the steps of:
(a) detecting an expression product of a gene in a first tissue sample suspected of a pathological condition wherein said expression product of said gene is represented by SEQ ID NO: 18 or a fragment thereof; and
(b) comparing expression of the expression product of the gene in the first tissue sample with expression of the expression product of the gene in a second tissue sample which is normal, wherein a difference in expression of the expression product of the gene in the first tissue sample relative to the second tissue sample identifies the first tissue sample as likely to be pathological or susceptible to a pathology.

5. The method according to claim 4, wherein the first and second tissue samples are from a human.

6. The method according to claim 5, wherein the first and second tissue samples are from the same human.


SEQUENCE LISTING (109 sequences)

SEQ ID NO: 1 - DNA, 1462 bp, Homo sapiens
ggccggctagagccgggggctgggcggggaccgggcttgtcggtgaagcggcagtggcggcggcggcggcggctcggcaggcgggttcaggcttcgggggccagccgccgccatgatcctgctggaggtgaacaaccgcatcatcgaggagacgctcgcgctcaagttcgagaacgcggccgccggaaacaaaccggaagcagtagaagtaacatttgcagatttcgatggggtcctctatcatatttcaaatcctaatggagacaaaacaaaagtgatggtcagtatttctttgaaattctacaaggaacttcaggcacatggtgctgatgagttattaaagagggtgtacgggagtttcttggtaaatccagaatcaggatacaatgtctctttgctatatgaccttgaaaatcttccggcatccaaggattccattgtgcatcaagctggcatgttgaagcgaaattgttttgcctctgtctttgaaaaatacttccaattccaagaagagggcaaggaaggagagaacagggcagttatccattatagggatgatgagaccatgtatgttgagtctaaaaaggacagagtcacagtagtcttcagcacagtgtttaaggatgacgacgatgtggtcattggaaaggtgttcatgcaggagttcaaagaaggacgcagagccagccacacagccccacaggtcctctttagccacagggaacctcctctggagctgaaagacacagacgccgctgtgggtgacaacattggctacattacctttgtgctgttccctcgtcacaccaatgccagtgctcgagacaacaccatcaacctgatccacacgttccgggactacctgcactaccacatcaagtgctctaaggcctatattcacacacgtatgcgggcgaaaacgtctgacttcctcaaggtgctgaaccgcgcacgcccagatgccgagaaaaaagaaatgaaaacaatcacggggaagacgttttcatcccgctaatcttgggaataagaggaggaagcggctggcaactgaaggctggaacacttgctactggataatcgtagcttttaatgttgcgcctcttcaggttcttaagggattctccgttttggttccattttgtacacgtttggaaaataatctgcagaaacgagctgtgcttgcaaagacttcatagttcccaagaattaaaaaaaaaaaaaaaagaattccacttgatcaacttaattccttttctttatcttccctccctcacttcccttttctcccaccctcttttccaagctgtttcgctttgcaatatattactggtaatgagttgcaggataatgcagtcataacttgttttctcctaagtatttgagttcaaaactcctgtatctaaagaaatacggttggggtcattaataaagaaaatctttctatcttaaaaaaaaaaaaaaa

SEQ ID NO: 2 - protein, 300 aa, Homo sapiens
MILLEVNNRIIEETLALKFENAAAGNKPEAVEVTFADFDGVLYHISNPNGDKTKVMVSISLKFYKELQAHGADELLKRVYGSFLVNPESGYNVSLLYDLENLPASKDSIVHQAGMLKRNCFASVFEKYFQFQEEGKEGENRAVIHYRDDETMYVESKKDRVTVVFSTVFKDDDDVVIGKVFMQEFKEGRRASHTAPQVLFSHREPPLELKDTDAAVGDNIGYITFVLFPRHTNASARDNTINLIHTFRDYLHYHIKCSKAYIHTRMRAKTSDFLKVLNRARPDAEKKEMKTITGKTFSSR

SEQ ID NO: 3 - DNA, 7633 bp, Homo sapiens
gggccggggggcggagccttgcgggctggagcgaaagaatgcgggggctgagcgcagaagcggctcgaggctggaagaggatcttgggcgccgccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcggggggttggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 4 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 5 - DNA, 1627 bp, Homo sapiens
gcccccaccatcaaggggaagaaagtgctcttcggattccggttcgccctggcctcccgcagccgccgcgggaccggcccccagcacacccccggggcgccgggcgcggggcagccgcccggacgcgcgcgggcctcaggcgccgccgggaccccagccccccaaactttggcaagttgcgggcgccgagcgcacccggaggcgcggggcgcggccgcaggcggagccgccccctgacgccgggccgccccctcccggccccggccgccccgccggctccgcggaaagtttgcggccgcccctgcgccgcacccggggcctgggtgagactgcggcggcggcagggcgcggacggccatatttgccggcgcggcccgagccgccgacaacaaaaagtgcgcgggcgctcggcgggcgctcggacgggcgcggggctgcagcgctaccgcccggcctcgccgccgccgccgccgccctcgcggcctggccccgccgcgcccggcgcgcccgccgcccggggggatgtcttacaaaccgaacttggccgcgcacatgcccgccgccgccctcaacgccgctgggagtgtccactcgccttccaccagcatggcaacgtcttcacagtaccgccagctgctcagtgactacgggccaccgtccctaggctacacccagggaactgggaacagccaggtgccccaaagcaaatacgcggagctgctggccatcattgaagagctggggaaggagatcagacccacgtacgcagggagcaagagtgccatggagaggctgaagcgcggcatcattcacgctagaggactggttcgggagtgcttggcagaaacggaacggaatgccagatcctagctgccttgttggttttgaaggatttccatctttttacaagatgagaagttacagttcatctcccctgttcagatgaaacccttgttttcaaaatggttacagtttcgtttttcctcccatggttcacttggctctgaacctacagtctcaaagattgagaaaagattttgcagttaattaggatttgcattttaagtagttaggaactgcccaggttttttttgttttttaagcattgatttaaaagatgcacggaaagttatcttacagcaaactgtagtttgcctccaagacaccattgtctccctttaatcttctcttttgtatacatttgttacccatggtgttctttgttccttttcataagctaataccactgtagggattttgttttgaacgcatattgacagcacgctttacttagtagccggttcccatttgccatacaatgtaggttctgcttaatgtaacttcttttttgcttaagcatttgcatgactattagtgcttcaaagtcaatttttaaaaatgcacaagttataaatacagaagaaagagcaacccaccaaacctaacaaggacccccgaacactttcatactaagactgtaagtagatctcagttctgcgtttattgtaagttgataaaaacatctggaagaaaatgactaaaactgtttgcatctttgtatgtatttattacttgatgtaataaagcttattttcattaacaatttgtattaaaaaaaaaaaaaaaa

SEQ ID NO: 6 - protein, 115 aa, Homo sapiens
MSYKPNLAAHMPAAALNAAGSVHSPSTSMATSSQYRQLLSDYGPPSLGYTQGTGNSQVPQSKYAELLAIIEELGKEIRPTYAGSKSAMERLKRGIIHARGLVRECLAETERNARS

SEQ ID NO: 7 - DNA, 5921 bp, Homo sapiens
agcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccccgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggaccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgccaaaggcgatgctggtccccctgggcctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccccggtcccatgggcccccctggattggctggaccccctggtgaatctggacgtgagggggctcctgctgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcccgtcggccccgtcggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggaagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctcaagggctccaacgagatcgagatccgcgccgagggcaacagccgcttcacctacagcgtcactgtcgatggctgcacgagtcacaccggagcctggggcaagacagtgattgaatacaaaaccaccaagtcctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaacccccccaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaaaaagaataaataaataagtttttaaaaaaggaagcttggtccacttgcttgaagacccatgcgggggtaagtccctttctgcccgttgggttatgaaaccccaatgctgccctttctgctcctttctccacaccccccttggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcatcccttgtgccgcaggctgggcgggagagactgttctgttctgttccttgtgtaactgtgttgctgaaagactacctcgttcttgtcttgatgtgtcaccggggcaactgcctgggggcggggatgggggcagggtggaagcggctccccatttttataccaaaggtgctacatctatgtgatgggtggggtggggagggaatcactggtgctatagaaattgagatgcccccccaggccagcaaatgttcctttttgttcaaagtctatttttattccttgatattttttctttctttttttttttttttgtggatggggacttgtgaatttttctaaaggtgctatttaacatgggaggagagcgtgtgcgctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccctaggggtgggaggaagcaaaagactctgtacctattttgtatgtgtataataatttgagatgtttttaattattttgattgctggaataaagcatgtggaaatgacccaaacataa

SEQ ID NO: 8 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPAAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 9 - DNA, 6447 bp, Homo sapiens
aggtctccgcttggagccgccgcacccgggacggtgcgtatcgctggaagtccggccttccgagagctagctgtccgccgcggcccccgcacgccgggcagccgtccctcgcgcctcgggcgcgccaccatggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgcaggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccccgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgccagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccaggactgcgtggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggacaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataagaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgcttcattgacctctacttgctgaaggagaaaaagacagcgataagcttcaatagtggcataccaaatggcacttttgatgaaataaaatatcaatattttctgcaatccaatgcactgatgtgtgaagtgagaactccatcagaaaaccaaagggtgctaggaggtgtgggtgccttccatactgtttgcccattttcattcttgtattataattaattttctacccccagagataaatgtttgtttatatcactgtctagctgtttcaaaatttaggtcccttggtctgtacaaataatagcaatgtaaaaatggttttttgaacctccaaatggaattacagactcagtagccatatcttccaaccccccagtataaatttctgtctttctgctatgtgtggtactttgcagctgcttttgcagaaatcacaattttcctgtggaataaagatggtccaaaaatagtcaaaaattaaatatatatatatattagtaatttatatagatgtcagcaattaggcagatcaaggtttagtttaacttccactgttaaaataaagcttacatagttttcttcctttgaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagctt

SEQ ID NO: 10 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 11 - DNA, 1207 bp, Homo sapiens
gagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagtttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaagttgcagctgttttgttgacattctgaatgcttctaagtaaatacaattttttttattaaaaaaaaaaaaaaaaaaaaaaaaa

SEQ ID NO: 12 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 13 - DNA, 2227 bp, Homo sapiens
taattcagaattgagtaaagaaatattttttctagtccttcatatattgaaaacttgccacatgacattgtatcgtcttcattttccagaagatgcgttggtgtgccataggtttctaacttccttgaaaatagttttttaagtcaattgtaaatatacgtattattgttaaaagtaactttaaactgcaacacatagcttcaaaacaatatagagattttgtaataccttataagtggagttggctaaaataccttatccatataaaacttattctattctttgcatgcttattttgtgtgttggttgctagcttaaagtttgatttgttgttactctttgtgtgccaaattcactaggcaagcggatttttcctcagacttcaaaaaataattcttttaagaaaaaatgtaaaaatgtttattctaaaaagctgcattaaagggacaacctataaaaagttttgctagctcatctttagaaggaagaaagaatattagcttgggtgatgtttaatttgggtggcgatagtttctgtaggctaaactttatgagaaaagtgtacctactctataaaggtaataaatgtaaaacctcttgctgttattgaggaagctcttcaactaccctaaatttcacaaatgtaacttataacactatgaaaagatttgaccaacaatttacgtttgctgtgtgctttagtttttgtttaagcatattcttttgcttgaatttctgtgttcatgagagttagggtgttttatgcttcttgaactaattttataacatatttaatatattaccagttaagatataaaatcatttgtacatagcgaattgtaaagcagctattaaagtaggtgaaataaagtatatatttgccggttatccatatcttttagaagtcctgacagaacaaccagtttatttgcacataggtagcttctgtttgaaggaaggtaaagttataaggaaactcaaatactataagatgtgtcaaggtatttctccagaattaattgcaaagctagtgctgaaggattttaatcagcttctaaaattttcttctcaataagacatatgttttgattacttagggaagattcctcatttttatttgccctttatgcatttaatccacatgataggacattaaaaattaatataaagaaaaatcgtgctcatactgtacatctatttctgtgcttggaactacttgttaatagtttttatcgaagctgtcagcaataagggacataaaactgctgtattatacattgtggaattgaataaacagcctaatttttttttttctagtatagggtacttaagcatttccacttttggaagaaaagtgtattagtattttatattgcatttcatttaaaaggacagttttttttttttttgtaaatccattcattgaaatggtttctaaactgtataatgtaatttggagcctatttagtaatagaattaaatgtcctatgtagtgctacaatttttgaattagaaagtgatcaaatgtaagaaaaaaatttaaaaattcagcccagaaaacaaaatagtgtattaaattagtttaatgtaaaaggaatttataagatttttttcctcaatatagatacctcacttgaaaagaaagcacagcatacttaaagtagttctagtaaacatgtcctagaaaacagttgctaaatgtaggacatcttttgaggaattagtttatgagaaataaaattttacttgtttttactatcctgttagaagtatttgtttatcctgataattttaagccaacatagtagtcttaaattacttttgaatttctaatctgtgaaggcagtaaatgaaatatctgttctgcaactgttgaaacaaataattggctacattgaccataattaaagttaaaattttgccaatgatgtacagttttatggttaaagttgctgtggttggttgcattacatgacacagaaaactgtcctctacctcacgtgaaataaatattttatatggttttactaaaaataagactcatgtatctggtcacctagtttacaaattttgaattatatttattgaaacatgacatactgtgctctgagcttatacctcaattgtattttgtgctgttttccattttcatgccttgtaaataacttgtatagattgtggatcaaatactaaataaaaacttttaatgccaaaaaaaaaaaaaaaa

SEQ ID NO: 14 - DNA, 2227 bp, Homo sapiens; identical to SEQ ID NO: 13

SEQ ID NO: 15 - DNA, 3416 bp, Homo sapiens
accaggcagcctgcgttcgccatgaagcgacccaaggagccgagcggctccgacggggagtccgacggacccatcgacgtgggccaagagggccagctgagccagatggccaggccgctgtccacccccagctcttcgcagatgcaagccaggaagaaacgcagagggatcatagagaaacggcgtcgagaccgcatcaacagtagcctttctgaattgcgacgcttggtccccactgcctttgagaaacagggctcttccaagctggagaaagccgaggtcttgcagatgacggtggatcacttgaaaatgctccatgccactggtgggacaggattctttgatgcccgagccctggcagttgacttccggagcattggttttcgggagtgcctcactgaggtcatcaggtacctgggggtccttgaagggcccagcagccgtgcagaccccgtccggattcgccttctctcccacctcaacagctacgcagccgagatggagccttcgcccacgcccactggccctttggccttccctgcctggccctggtctttcttccatagctgtccagggctgccagccctgagcaaccagctcgccatcctgggaagagtgcccagccctgtcctccccggtgtctcctctcctgcttaccccatcccagccctccgaaccgctccccttcgcagagccacaggcatcatcctgccagcccggaggaatgtgctgcccagtcgaggggcatcttccacccggagggcccgccccctagagaggccagcgacccctgtgcctgtcgcccccagcagcagggctgccaggagcagccacatcgctcccctcctgcagtcttcctccccaacaccccctggtcctacagggtcggctgcttacgtggctgttcccacccccaactcatcctccccagggccagctgggaggccagcgggagccatgctctaccactcctgggtctctgaaatcactgaaatcggggctttctgagctgccccttcaccaccccgccccaaggaataaggaaggttcttttaccaggagcccaaaaaagggcactgccttttctgctttgcttcgtggactggctcatatgtgaaggcacgttctccagccatcagaggccccctcctcctccaacccatctctccttctcactgttatcccagcttatccacccagctctcctggagctgttctggtctcagaggcttggttccatttctcacctgaacagatgagtcctgggagagaccctcagagatccgcccagacccctctcctgccctctgcacaccagcagcaggcatgaaccttgggtctgggaaaaagctttaacctgcagggcaccaggacccaaggcaggctgttccttggggcggtcagaccccagtcaggagcaatgactgactggctgcagccttcccacgccaagaggctggaacatagtgtctgcctcgcttcctggagatagtaactgagcaggggctacaaagaggtctcctgggaaccctgtctgccccttcccacctgtccttgggccacaccatcacactgaaccacaggacagaccctttctccaccacagccaaggcctggagactgggggcccagcagagcctgctcccaccctcctcccagcagcagacacccaccctctcactgactaacaggtccctgcacacagctggcctggtaaacccagctgggaggtttctaggcagcagcaaaactctgtgacagggtgtcctcacaccaggccttggacagctctcccagacaggagccagggttgagcaatggagagcccagcccccacgtcttacagtcgccatcctccaggcgtgtggtccctccccattgggtgcacagtgcagaggggccgtggccccatgtgatggtgcgcagagaggaacctcttgggattcagcaccagacgtctgtgctgcctggtttgcatccggctcacagagcccagactgctggaacagccaaggactgtcaggctggacaaaaataactgcaaggaggggcaagagaaaggatgattcgaggcaccttggcccttcaaggtcatgcagtgggtcgagcgcctgagatcctgttcaccaggactccacagagctggctctgctcagaagccatttcattccccggctccaccctaggccactttttctaacagaggaaacaaatggtccagcagtcgttcccagcagaacagcggagcctggactgacacccagtgggaccagtgttgccacaccagttgataaaatgcagaaacccttctgtactcgttggtaaatatctactcccccaagtgactccaggtgccccccaccgcctggcacttcccccaggactcctacgatctggttactgcctggccgatccaaggctgtggagtcccagagccagcagttcactggtgctcattccacactggttagatacttcagttgtcacccctgggaagattctcccacctcctccctttgatggaaccaccctccccagaggctgcattgaggagactccacagactgaaaagtgagtttgcagaaaccttggggaaaagggccctttcaaagaagtggataagagggaggagatcattgagtgacccagaaagctcttttgaaaagacagactcctcaaggagagataaagaggaaagcacctctttcattttttagtgtgagctaattccatcagactgctgtcctcctggacccatctgagatgtgcagtagcaaggagaggggggatcattttagagagtgggtcattggcagggagtgctccggagggaggcagaggggagactgtggtagaaggaagacagaactcacacatgctcccaggattggggacagggacagaggaggtaacagaaggcaaaggccagtttccccgttatcatgaaggggcccactcaggacaggaacaaggacaactcctcctcctcctcctcctctcctgctgctcctgggataccaggtcagtgatgtagtcttgcagtttggcaacttcctagcctgagaatccctagtggggctgtgggaaacacatttccacgttgcaagcatgcaactccaaagaatctgtgatgccactgaaatgagatgggaatgatccagctctttcagcatcttggttgaacttgctttcattgtccctgggatattgtggaaggaaaggtgactgtgtgatctgattctgtggtcaaggacttgcatcttgtgtttctatccccaagccttcctggtgtctccaactcctaccccattgcatgggttgttgcggacatccaataaagatttttttagtgcttctggaaaaaaaaaaaaaaaaaaaaaaaaaaaaaaaaaaaaaaaaaaaaaaaaaaaaaaacc

SEQ ID NO: 16 - protein, 328 aa, Homo sapiens
MKRPKEPSGSDGESDGPIDVGQEGQLSQMARPLSTPSSSQMQARKKRRGIIEKRRRDRINSSLSELRRLVPTAFEKQGSSKLEKAEVLQMTVDHLKMLHATGGTGFFDARALAVDFRSIGFRECLTEVIRYLGVLEGPSSRADPVRIRLLSHLNSYAAEMEPSPTPTGPLAFPAWPWSFFHSCPGLPALSNQLAILGRVPSPVLPGVSSPAYPIPALRTAPLRRATGIILPARRNVLPSRGASSTRRARPLERPATPVPVAPSSRAARSSHIAPLLQSSSPTPPGPTGSAAYVAVPTPNSSSPGPAGRPAGAMLYHSWVSEITEIGAF

SEQ ID NO: 17 - DNA, 1793 bp, Homo sapiens
aaaaccgcggttgccggagcccgaactgaggcggcggcgggagcccggttggcgtctggtcttcgcgtcggccccgcggagccagacgctgcccccggcgcggggagaagatggtgcctagcggcctcgggcccgccacgcgccgccacgagtgagcccagcgcgaccgcgggcgtccgccgagcagctggcccggctgggcccggggcgcgcagctgcccgccggggcggggtggagctgatcagaataatgttcagcatcaaccccctggagaacctgaaggtgtacatcagcagtcggcctcccctggtggtcttcatgatcagcgtaagcgccatggccatagctttcctgaccctgggctacttcttcaaaatcaaggagattaaatccccagaaatggcagaggattggaatacttttctgctacggttcaatgatttggacttgtgtgtatcagagaatgaaaccctcaagcatctcacaaacgacaccacaactccggaaagtacaatgaccagcgggcaggcccgagcttccacccagtccccccaggccctggaggactcgggcccggtgaatatctcagtctcaatcaccctaaccctggacccactgaaacccttcggagggtattcccgcaacgtcacccatctgtactcaaccatcttagggcatcagattggactttcaggcagggaagcccacgaggagataaacatcaccttcaccctgcctacagcgtggagctcagatgactgcgccctccacggtcactgtgagcaggtggtattcacagcctgcatgaccctcacggccagccctggggtgttccccgtcactgtacagccaccgcactgtgttcctgacacgtacagcaacgccacgctctggtacaagatcttcacaactgccagagatgccaacacaaaatacgcccaagattacaatcctttctggtgttataagggggccattggaaaagtctatcatgctttaaatcccaagcttacagtgattgttccagatgatgaccgttcattaataaatttgcatctcatgcacaccagttacttcctctttgtgatggtgataacaatgttttgctatgctgttatcaagggcagacctagcaaattgcgtcagagcaatcctgaattttgtcccgagaaggtggctttggctgaagcctaattccacagctccttgttttttgagagagactgagagaaccataatccttgcctgctgaacccagcctgggcctggatgctctgtgaatacattatcttgcgatgttgggttattccagccaaagacatttcaagtgcctgtaactgatttgtacatatttataaaaatctattcagaaattggtccaataatgcacgtgctttgccctgggtacagccagagcccttcaaccccaccttggacttgaggacctacctgatgggacgtttccacgtgtctctagagaaggattcctggatctagctggtcacgacgatgttttcaccaaggtcacaggagcattgcgtcgctgatggggttgaagtttggtttggttcttgtttcagcccaatatgtagagaacatttgaaacagtctgcacctttgatacggtattgcatttccaaagccaccaatccattttgtggattttatgtgtctgtggcttaataatcatagtaacaacaataataccttttcctccattttgcttgcaggaaacataccttaagttttttttgttttgtttttgtttttttgttttttgttttcctttatg

SEQ ID NO: 18 - protein, 314 aa, Homo sapiens
MFSINPLENLKVYISSRPPLVVFMISVSAMAIAFLTLGYFFKIKEIKSPEMAEDWNTFLLRFNDLDLCVSENETLKHLTNDTTTPESTMTSGQARASTQSPQALEDSGPVNISVSITLTLDPLKPFGGYSRNVTHLYSTILGHQIGLSGREAHEEINITFTLPTAWSSDDCALHGHCEQVVFTACMTLTASPGVFPVTVQPPHCVPDTYSNATLWYKIFTTARDANTKYAQDYNPFWCYKGAIGKVYHALNPKLTVIVPDDDRSLINLHLMHTSYFLFVMVITMFCYAVIKGRPSKLRQSNPEFCPEKVALAEA

SEQ ID NO: 19 - DNA, 5180 bp, Homo sapiens
accgggagcgcgcgctctgatccgaggagaccccgcgctcccgcagccatgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgacgggcaggatgcatgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaacatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccaatggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattctttttttttttttttttttttgctggtgtctgagcttcagtataaaagacaaaacttcctgtttgtggaacaaaagttcgaaagaaaaaacaaaacaaaaacacccagccctgttccaggagaatttcaagttttacaggttgagcttcaagatggtttttttggttttttttttttctctcatccaggctgaaggattttttttttctttacaaaatgagttcctcaaattgaccaatagctgctgctttcatattttggataagggtctgtggtcccggcgtgtgctcacgtgtgtatgcacgtgtgtgtgtccattagacacggctgacgtgtgtgcaaagtatccatgcggagttgatgctttgggaattggctcatgaaggttcttctcaagggtgcgagctcatccccctctctccttccttcttattgactgggagactgtgctctcgacagattcttcttgtgtcagaagtctagcctcaggtttctaccctcccttcacattggtggccaagggaggagcatttcatttggagtgattatgaatcttttcaagaccaaaccaagctaggacattaaaaaaaaaaaaaagaaaaagaaagaaaaaacaaaatggaaaaaggaaaaaaaaaaagaactgagatgacagagttttgagaatatatttgtaccatattta

SEQ ID NO: 20 - protein, 1370 aa, Homo sapiens [sequence omitted]

SEQ ID NO: 21 - DNA, 1155 bp, Homo sapiens
gccgctgccaccgcaccccgccatggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtgaaggtgccgagctataaacctccagaatattattagtctgcatggttaaaagtagtcatggataactacattacctgttcttgcctaataagtttcttttaatccaatccactaacactttagttatattcactggttttacacagagaaatacaaaataaagatcacacatcaagactatctacaaaaatttattatatatttacagaagaaaagcatgcatatcattaaacaaataaaatactttttatcacaaaaaaaaaaaaaaaaaaaaaaaaaaaaaaaaaaaaaaaaaaaaaaa

SEQ ID NO: 22 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 23 - DNA, 1336 bp, Homo sapiens
ggggcttgcagagccggcgccggaggagacgcacgcagctgactttgtcttctccgcacgactgttacagaggtctccagagccttctctctcctgtgcaaaatggcaactcttaaggaaaaactcattgcaccagttgcggaagaagaggcaacagttccaaacaataagatcactgtagtgggtgttggacaagttggtatggcgtgtgctatcagcattctgggaaagtctctggctgatgaacttgctcttgtggatgttttggaagataagcttaaaggagaaatgatggatctgcagcatgggagcttatttcttcagacacctaaaattgtggcagataaagattattctgtgaccgccaattctaagattgtagtggtaactgcaggagtccgtcagcaagaaggggagagtcggctcaatctggtgcagagaaatgttaatgtcttcaaattcattattcctcagatcgtcaagtacagtcctgattgcatcataattgtggtttccaacccagtggacattcttacgtatgttacctggaaactaagtggattacccaaacaccgcgtgattggaagtggatgtaatctggattctgctagatttcgctaccttatggctgaaaaacttggcattcatcccagcagctgccatggatggattttgggggaacatggcgactcaagtgtggctgtgtggagtggtgtgaatgtggcaggtgtttctctccaggaattgaatccagaaatgggaactgacaatgatagtgaaaattggaaggaagtgcataagatggtggttgaaagtgcctatgaagtcatcaagctaaaaggatataccaactgggctattggattaagtgtggctgatcttattgaatccatgttgaaaaatctatccaggattcatcccgtgtcaacaatggtaaaggggatgtatggcattgagaatgaagtcttcctgagccttccatgtatcctcaatgcccggggattaaccagcgttatcaaccagaagctaaaggatgatgaggttgctcagctcaagaaaagtgcagataccctgtgggacatccagaaggacctaaaagacctgtgactagtgagctctaggctgtagaaatttaaaaactacaatgtgattaactcgagcctttagttttcatccatgtacatggatcacagtttgctttgatcttcttcaatatgtgaatttgggctcacagaatcaaagcctatgcttggtttaatgcttgcaatctgagctcttgaacaaataaaattaactattgtagtgcgaaaaaaaaaaaaaaaaaaaaaaaaaaaaa

SEQ ID NO: 24 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 25 - DNA, 543 bp, Homo sapiens
ccctcccccagagctgttatgatgagctgcaggaagctctggagttgggcggggagactggactcccgggagtacccagcgctgcttttttgttcatgcagcctgtgattcatagcttccctggggtgttggggagaatcacatttgggtcagccaggtttagcactgacagttttgtctttagaatcaagcagatgtggaatcaaatctggctgtatccatgaccaactctgaagccatgagtgggttacatagctttagagcctcagcatactcatctggaaagtggaagtgatcatgtctattttgcagagttgttgccacttttcctctctggaccccactttccccatctgtcagatgaaagagtgggatgagatggcctgtctatttatctctcaatcacaatggctctatttgaaaaaagtttgaactgccctaagtgctaaaaaggaagatggggagccatcaagacaaacttaggcctacattaccatcgagttcagagaatggcagaccgtaagcaccagccagcttcactcc

SEQ ID NO: 26 - DNA, 543 bp, Homo sapiens; identical to SEQ ID NO: 25

SEQ ID NO: 27 - DNA, 3350 bp, Homo sapiens
tggaggagagaagatggcggaagcggagtgagtgactagatgatttaaggaccatagtacagctatggatactgaaccaaacccgggaacatcttctgtgtcaacaacaaccagcagtaccaccaccaccaccatcaccacttcctcctctcgaatgcagcagccacagatctctgtctacagtggttcagaccgacatgctgtacaggtaattcaacaggcattgcatcggccccccagctcagctgctcagtaccttcagcaaatgtatgcagcccaacaacagcacttgatgctgcatactgcagctcttcagcagcagcatttaagcagctcccagcttcagagccttgctgctgttcaggcaagtttgtccagtggaagaccatctacatctcccacaggaagtgtcacacagcagtcaagtatgtcccaaacgtctatcaacctctccacttctcctacacctgcacagttaataagccgttcccaggcttccagttctaccagcggcagtattacccaacagactatgttactagggagtacttcccctaccctaacggcaagccaagctcaaatgtatctccgagctcaaatgctgattttcacacccgctaccactgtggctgctgtacagtctgacattcctgttgtctcgtcgtcatcgtcatcttcctgtcagtctgcagctactcaggttcagaatttaacattacgcagccagaagttgggtgtattatctagctcacagaatggtccaccaaaaagcactagtcaaactcagtcattgacaatttgtcataacaaaacaacagtgaccagttctaaaatcagccaacgagatccttctccagaaagtaataagaaaggagagagcccaagcctggaatcacgaagcacagctgtcacccggacatcaagtattcaccagttaatagcaccagcttcatattctccaattcagcctcattctctaataaaacatcagcagattcctcttcattcaccaccttccaaagtttcccatcatcagctgatattacaacagcagcaacagcaaattcagccaatcacacttcagaattcaactcaagacccacccccatcccagcactgtataccactccagaaccatggccttcctccagctcccagtaatgcccagtcacagcattgttcaccgattcagagtcatccctctcctttaacagtgtctcctaatcagtcacagtcagcacagcagtctgtagtggtgtctcctccaccacctcattcaccaagtcagtctcctactataattattcatccacaagcacttattcagccacaccctcttgtgtcatcagctctccagccagggccaaatttgcagcagtccactgctaatcaggtgcaagctacagcacagttgaatcttccatcccatcttccacttccagcttcccctgttgtacacattggcccagttcagcagtctgccttggtatccccaggccagcagattgtctctccatcacaccagcaatattcatccctgcagtcctctccaatcccaattgcaagtcctccacagatgtcgacatctcctccagctcagattccaccactgcccttgcagtctatgcagtctttacaagtgcagcctgaaattctgtcccagggccaggttttggtgcagaatgctttggtgtcagaagaggaacttccagctgcagaagctttggtccagttgccatttcagactcttcctcctccacagactgttgcggtaaacctacaagtgcaaccaccagcacctgttgatccaccagtggtttatcaggtagaagatgtgtgtgaagaagaaatgccagaagagtcagatgaatgtgtccggatggatagaaccccaccaccacccactttgtctccagcagctataacagtggggagaggagaagatttgacttctgaacatcctttgttagagcaagtggaattacctgctgtggcatcagtcagtgcttcagtaattaaatctccatcagatccctcacatgtttctgttccaccacctccattgttacttccagctgccaccacaaggagtaacagtacatctatgcacagtagcattcccagtatagagaacaaacctccacaggctattgttaaaccacagatcctaacccatgttattgaaggctttgtgattcaggagggattggagccatttcctgtgagtcgttcctctttgctaatagaacagcctgtgaaaaaacggcctcttttggataatcaggtgataaattcagtgtgtgttcagccagagctacagaataatacaaaacatgcggataattcatctgacacagagatggaagacatgattgctgaagagacattagaagaaatggacagtgagttgctcaagtgtgaattctgtgggaaaatgggatatgctaatgaatttttgcggtcaaaacgattctgcactatgtcatgtgccaaaaggtacaatgttagctgttctaaaaaatttgcacttagtcgttggaatcgtaagcctgataatcaaagtcttgggcatcgtggccgtcgtccaagtggccctgatggggcagcgagagaacatatccttaggcagcttccaattacttatccatctgcagaagaagacttggcttctcatgaagattctgtgccatctgctatgacaactcgtctgcgcaggcagagcgagcgggaaagagaacgtgagcttcgggatgtgagaattcggaaaatgcctgagaacagtgacttgctaccagttgcacaaacagagccatctatatggacagttgatgatgtctgggccttcatccattctttgcctggctgccaggatatcgcagatgaattcagagcacaggagattgatggacaggcccttctcttgctgaaagaagaccatctcatgagtgcaatgaatatcaagctaggcccagccctgaagatctgtgcacgcatcaactctctgaaggaatcttaacaggaacatgaagccttgataaaacagcagttttacttttctcacaaaaacttgtaaggtaaaggcctaacttggtctagaatatgacacttattgtggtggatagccaagcacattgggatctccacatcaaatactgacatttcttctacaggtataataattcatcatgcattttcataattaataaacattggtaaaattaattttacaggttacatgaaacattgaaagacttgttacagagggccatgatatttttcaaagaaatgtgttatactagataatttttttaaaggtgatgtttatcattaatataaagaatccttttaaa

SEQ ID NO: 28 - protein, 983 aa, Homo sapiens
MDTEPNPGTSSVSTTTSSTTTTTITTSSSRMQQPQISVYSGSDRHAVQVIQQALHRPPSSAAQYLQQMYAAQQQHLMLHTAALQQQHLSSSQLQSLAAVQASLSSGRPSTSPTGSVTQQSSMSQTSINLSTSPTPAQLISRSQASSSTSGSITQQTMLLGSTSPTLTASQAQMYLRAQMLIFTPATTVAAVQSDIPVVSSSSSSSCQSAATQVQNLTLRSQKLGVLSSSQNGPPKSTSQTQSLTICHNKTTVTSSKISQRDPSPESNKKGESPSLESRSTAVTRTSSIHQLIAPASYSPIQPHSLIKHQQIPLHSPPSKVSHHQLILQQQQQQIQPITLQNSTQDPPPSQHCIPLQNHGLPPAPSNAQSQHCSPIQSHPSPLTVSPNQSQSAQQSVVVSPPPPHSPSQSPTIIIHPQALIQPHPLVSSALQPGPNLQQSTANQVQATAQLNLPSHLPLPASPVVHIGPVQQSALVSPGQQIVSPSHQQYSSLQSSPIPIASPPQMSTSPPAQIPPLPLQSMQSLQVQPEILSQGQVLVQNALVSEEELPAAEALVQLPFQTLPPPQTVAVNLQVQPPAPVDPPVVYQVEDVCEEEMPEESDECVRMDRTPPPPTLSPAAITVGRGEDLTSEHPLLEQVELPAVASVSASVIKSPSDPSHVSVPPPPLLLPAATTRSNSTSMHSSIPSIENKPPQAIVKPQILTHVIEGFVIQEGLEPFPVSRSSLLIEQPVKKRPLLDNQVINSVCVQPELQNNTKHADNSSDTEMEDMIAEETLEEMDSELLKCEFCGKMGYANEFLRSKRFCTMSCAKRYNVSCSKKFALSRWNRKPDNQSLGHRGRRPSGPDGAAREHILRQLPITYPSAEEDLASHEDSVPSAMTTRLRRQSERERERELRDVRIRKMPENSDLLPVAQTEPSIWTVDDVWAFIHSLPGCQDIADEFRAQEIDGQALLLLKEDHLMSAMNIKLGPALKICARINSLKES

SEQ ID NO: 29 - DNA, 2133 bp, Homo sapiens
cgggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaatccaccaaccagaacgtggtttgcctgaggctgtaactgagagaaagattctggggctgtcttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctcccctttgttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatctttcaaaataaaaagtaatgacttagaaactgaaaaaaaaaaaaaaaaaaaaaaaaaaaaaa

SEQ ID NO: 30 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 31 - DNA, 634 bp, Homo sapiens
gccagccctcggaaacgcgaagtgagcggcggggtcgactgacggtaacggggcagagaggctgttcgcagagctgcggaagatgaatgccagaggacttggatctgagctaaaggacagtattccagttactgaactttcagcaagtggaccttttgaaagtcatgatcttcttcggaaaggtttttcttgtgtgaaaaatgaacttttgcctagtcatccccttgaattatcagaaaaaaatttccagctcaaccaagataaaatgaatttttccacactgagaaacattcagggtctatttgctccgctaaaattacagatggaattcaaggcagtgcagcaggttcagcgtcttccatttctttcaagctcaaatctttcactggatgttttgaggggtaatgatgagactattggatttgaggatattcttaatgatccatcacaaagcgaagtcatgggagagccacacttgatggtggaatataaacttggtttactgtaatagtgtgctgttcatggaaaccgagggctgcatcttgtttatagtcatctttgtactgtaatttgatgtacacaacattaaaagtactgacacctgaaaaaaaaaaaaaaaaaaaaaaaaaaaaaa

SEQ ID NO: 32 - protein, 141 aa, Homo sapiens
MNARGLGSELKDSIPVTELSASGPFESHDLLRKGFSCVKNELLPSHPLELSEKNFQLNQDKMNFSTLRNIQGLFAPLKLQMEFKAVQQVQRLPFLSSSNLSLDVLRGNDETIGFEDILNDPSQSEVMGEPHLMVEYKLGLL

SEQ ID NO: 33 - DNA, 1591 bp, Homo sapiens
gtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaaaaaaaaaaaaaaaaaaaaaaaaaaaaa

SEQ ID NO: 34 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 35 - DNA, 475 bp, Homo sapiens
  misc_feature (434)..(434): n is a, c, g, or t
gtacgggagtttcttggtaaatccagaatcaggatacaatgtctctttgctatatgaccttgaaaatcttccggcatccaaggattccattgtgcatcaagctggcatgttgaagcgaaattgttttgcctctgtctttgaaaaatacttccaattccaagaagagggcaaggaaggagagaacagggcagttatccattatagggatgatgagaccatgtatgttgagtctaaaaaggacagagtcacagtagtcttcagcacagtgtttaaggatgacgacgatgtggtcattggaaaggtgttcatgcaggagttcaaagaaggacgcagagccagccacacagccccacaggtcctctttagccacagggaacctcctctggagctgaaagacacagacgccgctgtgggtgacaacattggctacattnacctttgngctgttccntngtacaccantgccagngctnga

SEQ ID NO: 36 - DNA, 531 bp, Homo sapiens
  misc_feature (438)..(438): n is a, c, g, or t
gtacaaaatggaaccaaaacggagaatcccttaagaacctgaagaggcgcaacattaaaagctacgattatccagtagcaagtgttccagccttcagttgccagccgcttcctcctcttattcccaagattagcgggatgaaaacgtcttccccgtgattgttttcatttcttttttctcggcatctgggcgtgcgcggttcagcaccttgaggaagtcagacgttttcgcccgcatacgtgtgtgaatataggccttagagcacttgatgtggtagtgcaggtagtcccggaacgtgtggatcaggttgatggtgttgtctcgagcactggcattggtgtgacgagggaacagcacaaaggtaatgtagccaatgttgtcacccacagcggcgtctgtgtctttcagctccagaggaggttcccagtggctaaagangacctgtggggctgtgtggctggctctgcgtccttctttgaactcctgcatgaacacctttccatgaccacatcgtcgtcatccttaacactg

SEQ ID NO: 37 - DNA, 345 bp, Homo sapiens
  misc_feature (6)..(6): n is a, c, g, or t
agggtnggccnacagagtataatcaagggntttttttcaacattaatcaanaaattcctccatntgcaaaagtcagcctatgccattaaatacagccnagatcataaaatacaaangncattagggctactgaatcttganattcattcactaaaatacccngggaaaaggaaaaagtttgcccttgngaaaggggggnatcattttttaaaaaancttttgaaagccncaaacatctgggagngggctaaaagccgntacaaaggnattttatacaatctatgctgagtcagctaggagttaacaggaggctggatgcnnatggnaaaanatacccccgcgtac

SEQ ID NO: 38 - DNA, 447 bp, Homo sapiens
  misc_feature (434)..(434): n is a, c, g, or t
gtacgcggggagcaagagtgccatggagaggctgaagcgcggcatcattcacgctagaggactggttcgggagtgcttggcagaaacggaacggaatgccagatcctagctgccttgttggttttgaaggatttccatctttttacaagatgagaagttacagttcatctcccctgttcagatgaaacccttgttttcaaaatggttacagtttcgtttttcctcccatggttcacttggctctgaacctacagtctcaaagattgagaaaagattttgcagttaattaggatttgcattttaagtagttaggaactgcccaggttttttttgttttttaagcattgatttaaaagatgcacggaaagttatcttacagcaaactgtagtttgcctccaagacaccattgtctccctttaatcttctcttttgnatacatttggtac

SEQ ID NO: 39 - DNA, 289 bp, Homo sapiens
agagtcttttgcttcctcccacccctagggggaaaaactgctttgtgctttgggaagttgtctctgaaacccggggacagaggacgcaggacagactaggagggagccgggaggatgggctgcagctgtggaggagggtttcagaggagagaggtcggagagcagaggcctgagaagccagaggcaggtggagagagggtggaaagtgagcagcgggctgggctggagccgcacacgctctcctcccatgttaaatagcacctttagaaaaattcacaagtccccatcc

SEQ ID NO: 40 - DNA, 393 bp, Homo sapiens
acagctatgacctgattacgccaagcttggtaccgagctcggatccactagtaacggccgccagtgtgctggaattcgcccttagcggccgcccgggcaggtacagagtcttttgcttcctcccacccctagggggaaaaactgctttgtgctttgggaagttgtctctgaaacccggggacagaggacgcaggacagactaggagggagccgggaggatgggctgcagctgtggaggagggtttcagaggagagaggtcggagagcagaggcctgagaagccagaggcaggtggagagagggtggaaagtgagcagcgggctgggctggagccgcacacgctctcctcccatgttaaatagcacctttagaaaaattcacaagtccccatcc

SEQ ID NO: 41 - DNA, 221 bp, Homo sapiens
  misc_feature (4)..(4): n is a, c, g, or t
agantcttttgcttcctcccacccctanggggaaaaactgctttgtgctttgggaagttgtctctgaaacccggggacagaggacgcaggacagactaggagggagccgggaggatgggctgcagctgtggaggagggtttcagaggagagaggtcggagagcagaggcctgagaagccagaggcaggtggagaganggtggaaagtgancancgggctgg

SEQ ID NO: 42 - DNA, 271 bp, Homo sapiens
  misc_feature (4)..(4): n is a, c, g, or t
agantcttttgcttcctcccacccctanggggaaaaactgctttgtgctttgggaagttgtctctgaaacccggggacagangacgcaggacagactagganggagccgggangatgggctgcagctgtggagganggtttcagangaganangtcggagancagaggcctgagaagccagangcaggtggagaganggtggaaagtgagcagcgggctgggctggagccgcacacgctctcctnccatgttaaatagcacctttanaaaa

SEQ ID NO: 43 - DNA, 255 bp, Homo sapiens
gcgggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcataccttagatta

SEQ ID NO: 44 - DNA, 147 bp, Homo sapiens
gcgggagggcagattcggaccactaggcctgaaatgacatttcactaaaagtctccaaaacatttctaagactactaaggccttttatgtaatttctttaaatgtgtatttcttaagaattcaaatttgtaataaaactatttgtat

SEQ ID NO: 45 - DNA, 144 bp, Homo sapiens
ggagggcagattcggaccactaggcctgaaatgacatttcactaaaagtctccaaaacatttctaagactactaaggccttttatgtaatttctttaaatgtgtatttcttaagaattcaaatttgtaataaaactatttgtat

SEQ ID NO: 46 - DNA, 246 bp, Homo sapiens
aaggtatgcaaagcttacaaatttattagcagcttgtgcagtaagtttcttttaatgcgtctgtggcaatactagcaacaaattaataaaagcttaatttttatacaaatagttttattacaaatttgaattcttaagaaatacacatttaaagaaattacataaaaggccttagtagtcttagaaatgttttggagacttttagtgaaatgtcatttcaggcctagtggtccgaatctgccctcc

SEQ ID NO: 47 - DNA, 484 bp, Homo sapiens
  misc_feature (22)..(22): n is a, c, g, or t
gcaaagcttacaaatttattancagcttgngcagtaagtttcttttaatgcgtctgnggcaatactagcaacaaattaataaaagcttaatttttatacaaatagttttattacaaatttgaattcttaagaaatacncatttaaagaaattacataaaaggccttantagtcttaaaaatgntttggagacttttantgaaatgncatttcaggcctagtggnccgaatctgccctcctgcggnccatgcgatgccctgctgaggnctgtgaacacagctcatganaaaccacggaaatggcccgaatgngcttacgtgngaaaatactgatactgggattcaacagagctgtttttcaagccaggatgcagaatgaggaatactaatgaaatgacggcctttaagggtgttgcttttgaagtcaagtcattcagtttgngattagtgnttaaaaccctgaaaatatttaatacngaataaaa

SEQ ID NO: 48 - DNA, 246 bp, Homo sapiens
ggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcatacctt

SEQ ID NO: 49 - DNA, 239 bp, Homo sapiens
  misc_feature (57)..(57): n is a, c, g, or t
gcaaagcttacaaatttattaacagcttgggcagtaagtttcttttaatgcgtctgnggnaatactagcaacaaattaataaaagcttaatttttatacncatagttttattacaaatttgaattcttaanaaatacncatttaaagaaattacntaaaaggncttantagtcttaaaaatgntttgganacttttantgaaatgncatttcaggcctannggnccgaatctgccctcc

SEQ ID NO: 50 - DNA, 246 bp, Homo sapiens; identical to SEQ ID NO: 48

SEQ ID NO: 51 - DNA, 242 bp, Homo sapiens
gcgggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgc

SEQ ID NO: 52 - DNA, 254 bp, Homo sapiens
gtacgcgggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcatacctta

SEQ ID NO: 53 - DNA, 245 bp, Homo sapiens
gcgggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcata

SEQ ID NO: 54 - DNA, 249 bp, Homo sapiens
gcgggagggcagattcggaccactaggcctgaaatgacatttcactaaaagtctccaaaacatttctaagactactaaggccttttatgtaatctctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcatacctt

SEQ ID NO: 55 - DNA, 246 bp, Homo sapiens; identical to SEQ ID NO: 48

SEQ ID NO: 56 - DNA, 239 bp, Homo sapiens
ggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgc

SEQ ID NO: 57 - DNA, 476 bp, Homo sapiens
ggcactgggcgactctgtggctcgctgaggaaaaaaagaactaaacatgggcaaaggagatctagggaagctgagaggtaaaatgtgatatgcattctttgtccacacttgttgggaggagcacaagaagaagcacccagatgctcagtcagctcctcagagttttctaagaaatgctcagagaggtgcaagaccatgtctgctaaagagaaagtgagatttgaagacatgtcaaagatggacaagacccattatgaaagagaaatgaaaacctatatccttcctaaatgggagacaaaaaagaagttcgaggatcccaatgcacccaagaggcctccttcggccttcttcctcttctgctctgagtatcgcccaaaaatcaaaggagaacatcctggcctgtccattggtgatgttgcgaaaaactgggagagatgtggaataacactgctgcagacgacagcagccttatgaaa

SEQ ID NO: 58 - DNA, 267 bp, Homo sapiens
gtacaaatgattttatatcttaactggtaatatattaaatatgttataaaattagttcaagaagcataaaacaccctaactctcatgaacacagaaattcaagcaaaagaatatgcttaaacaaaaactaaagcacacagcaaacgtaaattgttggtcaaatcttttcatagtgttataagttacatttgtgaaatttagggtagttgaagagcttcctcaataacagcaagaggttttacatttattacctttatagagtaggta

SEQ ID NO: 59 - DNA, 477 bp, Homo sapiens
  misc_feature (459)..(459): n is a, c, g, or t
tcgttggtaaatacctactcccccaagtgactccaggtgccccccaccgcctggcacttcccccaggactcctacgatctggttactgcctggccgatccaaggctgtggagtcccagagccagcagttcactggtgctcattccacactggttagatacttcagttgtcacccctgggaagattctcccacctcctccctttgatggaaccaccctccccagaggctgcattgaggagactccacagactgaaaagtgagtttgcagaaaccttggggaaaagggccctttcaaagaagtggataagagggaggagatcattgagtgacccagaaagctcttttgaaaagacagactcctcaaggagagataaagaggaaagcacctctttcattttttagtgtgagctaattccatcagactgctgtcctcctggacccatctgagatgtgcagtancanggagaggggggatca

SEQ ID NO: 60 - DNA, 405 bp, Homo sapiens
  misc_feature (391)..(391): n is a, c, g, or t
ccaagattacaatcctttctggtgttataagggggccattggaaaagtctatcatgctttaaatcccaagcttacagtgattgttccagatgatgaccgttcattaataaatttgcatctcatgcacaccagttacttcctctttgtgatggtgataacaatgttttgctatgctgttatcaagggcagacctagcaaattgcgtcagagcaatcctgaattttgtcccgagaaggtggctttggctgaagcctaattccacagctccttgttttttgagagagactgagagaaccataatccttgcctgctgaacccagcctgggcctggatgctctgtgaatacattatcttgcgatgttgggttattccagccaaagacatttcaggngcctgtaactgatt

SEQ ID NO: 61 - DNA, 300 bp, Homo sapiens
  misc_feature (5)..(5): n is a, c, g, or t
cctancttggtttggtcttgaaaanattcataatcactccaaatgaaatgctcctcccttggccaccaatgngaagggagggtaaaaacctgaggctanacttntgacacaanaaaaatntgtcganagcacagtctcccagtcaataaaaaggaaggananagggggatganctcncacccttgaaaaaaaccttnatgagccaattcccaaagcatcaactccgcatggatactttgcacacacatcagccgngtntaanggacacacacacgtgcatacncacgtgagcacacnccg

SEQ ID NO: 62 - DNA, 469 bp, Homo sapiens
  misc_feature (21)..(21): n is a, c, g, or t
gataaaaagtattttatttgnttaatgatatgcatgcttttcttctgtaaatatataataaatttttgtagatagtcttgatgngngatctttattttgtatttctctgggnaaaaccagngaatataactaaagngntagtggattggattaaaagaaacttattaggcaagaacaggnaatgnagttatccatgactacttttaaccatgcagactaataatattctggaggnttatagctcggcaccttcaccttttttcactggnatttcatgnaaggcatcaaccactgnaatttttgctgatgctgaagcctgnccttgggaattggatgcatggcactcatattctccagcatcttccttacttagaggagataccagcacccagccagttacttcatgcttttctgggccaccccgggtctgaatgggccaggttgtcccggncccangcaggagttctgn

SEQ ID NO: 63 - DNA, 463 bp, Homo sapiens
  misc_feature (412)..(412): n is a, c, g, or t
atggatgaaaactaaaggctcgagttaatcacattgtagtttttaaatttctacagcctagagctcactagtcacaggtcttttaggtccttctggatgtcccacagggtatctgcacttttcttgagctgagcaacctcatcatcctttagcttctggttgataacgctggttaatccccgggcattgaggatacatggaaggctcaggaagacttcattctcaatgccatacatcccctttaccattgttgacacgggatgaatcctggatagatttttcaacatggattcaataagatcagccacacttaatccaatagcccagttggtatatccttttagcttgatgacttcataggcactttcaaccaccatcttatgcacttccttccaattttcactatcattgncagttcccatttctggactcaattcctggagagaaacacctgccncattcc

SEQ ID NO: 64 - DNA, 442 bp, Homo sapiens
gttcatgcagcctgtgattcatagcttccctggggtgttggggagaatcacatttgggtcagccaggtttagcactgacagttttgtctttagaatcaagcagatgtggaatcaaatctggctgtatccatgaccagctctgaagccatgagtgggttacatagctttagggcctcagcatactcatctggaaagtggaagtgatcatgtctattttgcagagttgttgccacttttcctctctggaccccactttccccatctgtcagatgaaagagtgggatgagatggcctgtctatttatctctcaatcacaatggctctatttgaaaaaagtttgaactgccctaagtgttaaaaaggaagatggggagccatcaagacaaacttaggcctacattaccatcgagttcagagaatggcagaccggaagcaccagcca

SEQ ID NO: 65 - DNA, 461 bp, Homo sapiens
gcgggaaagagaacgtgagcttcgggatgtgagaattcggaaaatgcctgagaacagtgacttgctaccagttgcacaaacagagccatctatatggacagttgatgatgtctgggccttcatccattctttgcctggctgccaggatatcgcagatgaattcagagcacaggagattgatggacaggcccttctcttgctgaaagaagaccatctcatgagtgcaatgaatatcaagctaggcccagccctgaagatctgtgcacgcatcaactctctgaaggaatcttaacaggaacatgaagccttgataaaacggcagttttacttttctcacaaaaacttgtaaggtaaaggcctaacttggtctagaatatgacacttattgtggtggatagccaagcacattgggatctccacatcaaatactgacatttcttctacaggtataataattcatc

SEQ ID NO: 66 - DNA, 507 bp, Homo sapiens
  misc_feature (375)..(375): n is a, c, g, or t
cggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctataacttaactattaaggccttttccacacgcattaatagtcccatttttctcttgccatttgtagctttgcccattgtcctattggcacatgggtggacacggatctgctgggctctgccttaaacacacattgcagcttcaacttttctctttagtgttctgtttgaaactaatacttaccgagtcagactttgtgttcatttcatttcanggtcttggctgcctgtgggcttccccaggtggcctggaggtgggcaaagggaagtaacagacacacgatgttgtcaaggatggttttgggactagangctcagtggtgggagagatccctgcagaanccacc

SEQ ID NO: 67 - DNA, 543 bp, Homo sapiens
  misc_feature (492)..(492): n is a, c, g, or t
gtacatgttatagttcttctcgaagtcccgggccagcagctccacggggtggtctcctgcctccaggcgcttctcattctcatggatcttcttcacccgcagcttctgcttctcagtcagaaggttgttgtcctcatccctctcatacagggtgaccaggacgttcttgagccagtcccgcatgcgcagggggaattcggtcagctcagagtccaggcaaggggggatgtatttgcaaggcccgatgtagtccaggtggagcttgtggcccttcttggtgccctccagggtacactttgtggcaaagaagtggcaggaagagtcgaaggtcttgttgtcattgctgcacaccttctcaaactcgccaatgggggctgggcagctggtggggtcctggcacacgcacatgggggtgttgttctcatccagctcgcacaccttgccgtgtttgcagtggtggttctggcagggattttccgccaccacctcctnttnggtttcctctgcacatnatnaaattntnctanttncacctgganagga

SEQ ID NO: 68 - DNA, 394 bp, Homo sapiens
gggcttctcctcctctgtcttttggagaaccagggctcttctcaggggctctagggactgtcaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgaggactcgttcacctccaagcatttcatgaaaaagctgcttcttattaatcatacaaactctcaccatgatgtgaagagtttcacaaatctttcaaaataaaaagtaatgacttag

SEQ ID NO: 69 - DNA, 506 bp, Homo sapiens
ctgtcttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaactctcaccatgatg

SEQ ID NO: 70 - DNA, 414 bp, Homo sapiens
  misc_feature (359)..(359): n is a, c, g, or t
cggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgngttcatttcatttnagggtctggctgnctgggggcttccccagggggcctggagg

SEQ ID NO: 71 - DNA, 485 bp, Homo sapiens
  misc_feature (451)..(451): n is a, c, g, or t
aaagatgactataaacaagatgcagccctcggtttccatgaacagcacactattacagtaaaccaagtttatattccaccatcaagtgtggctctcccatgacttcgctttgtgatggatcattaagaatatcctcaaatccaatagtctcatcattacccctcaaaacatccagtgaaagatttgagcttgaaagaaatggaagacgctgaacctgctgcactgccttgaattccatctgtaattttagcggagcaaatagaccctgaatgtttctcagtgtggaaaaattcattttatcttggttgagctggaaatttttttctgataattcaaggggatgactaggcaaaagttcatttttcacacaagaaaaacctttccgaagaagatcatgactttcaaaaggtccacttgctgaaagttcagtaactggaatactgtcctttanctcagatccaagtcctctggcattcatctcccgc

SEQ ID NO: 72 - DNA, 362 bp, Homo sapiens
  misc_feature (5)..(5): n is a, c, g, or t
ttgcnggnaatatattgctgnactgngngtgngcnannnttattcaaggncatcgtgatgctgagaagttnccntgataacctgnccatctctagtttcaaccgtcttaatcaaaagtgtcctttttgagngggtatcaaccaaagggagtgaatccanattantttccctcaggttcagggaggaaaagtttggaagaggcagaaaaatcctggtctcctcgccttccancagcttcctgnaggnggcaatctcaatgncaagggccatccttaacatngancaggtcttggtattcacgaaggngacgagccatttcctccttcatattctgaanctcatcctgcaggcngncaatagtg

SEQ ID NO: 73 - DNA, 444 bp, Homo sapiens
  misc_feature (13)..(13): n is a, c, g, or t
aatatattgctgnactgngngtgngcnanttttattcaaggncatcgtgatgctgagaagttnccntgataacctgnccatctctagtttcaaccgtcttaatcaaaagtgtcctttttgagngggtatcaaccaaagggagtgaatccanattantttccctcaggttcagggaggaaaagtttggaagaggcagaaaaatcctggtctcctcgccttccancagcttcctgnaggnggcaatctcaatgncaagggccatccttaacatngancaggtcttggtattcacgaaggngacgagccatttcctccttcatattctgaanctcatcctgcaggcngncaatagtgncttggnagataccagattctacgggaaagttctntttcattnnacncatnnggnggnccagggactcatnggttncnttaanggcatcc

SEQ ID NO: 74 - DNA, 494 bp, Homo sapiens
  misc_feature (310)..(310): n is a, c, g, or t
tgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggntgaaactagagatggacaggntatcaacaaaacttctcagcatcacgatgaccttgaataaaattgccacactcagtgcagcatatattaccagcaagaataaaaagaaatccctatcttaagaacngctttcagngcctttctgcagttttcagnacncaaganaaatttgaatagganaagc

SEQ ID NO: 75 - DNA, 19 bp, Homo sapiens
gcaactgaaggctggaaca

SEQ ID NO: 76 - DNA, 20 bp, Homo sapiens
tgaagaggcgcaacattaaa

SEQ ID NO: 77 - DNA, 23 bp, Homo sapiens
agtgggtgaatgtggttatggcc

SEQ ID NO: 78 - DNA, 23 bp, Homo sapiens
ctggcactgctcaggatgtcttc

SEQ ID NO: 79 - DNA, 21 bp, Homo sapiens
gctggccatcattgaagagct

SEQ ID NO: 80 - DNA, 21 bp, Homo sapiens
tctccatggcactcttgctcc

SEQ ID NO: 81 - DNA, 17 bp, Homo sapiens
gagatcgagatccgcgc

SEQ ID NO: 82 - DNA, 19 bp, Homo sapiens
tgcagccatcgacagtgac

SEQ ID NO: 83 - DNA, 21 bp, Homo sapiens
ggcaccccatctgttgatcac

SEQ ID NO: 84 - DNA, 23 bp, Homo sapiens
ggtaaagaattttggtcccagaa

SEQ ID NO: 85 - DNA, 23 bp, Homo sapiens
tctaagaagtgctcagagaggtg

SEQ ID NO: 86 - DNA, 22 bp, Homo sapiens
ttcatttctctttcataacggg

SEQ ID NO: 87 - DNA, 21 bp, Homo sapiens
caaattcactaggcaagcgga

SEQ ID NO: 88 - DNA, 22 bp, Homo sapiens
ggttgtccctttaatgcagctt

SEQ ID NO: 89 - DNA, 21 bp, Homo sapiens
tctgagctgccccttcaccac

SEQ ID NO: 90 - DNA, 23 bp, Homo sapiens
acgtgccttcacatatgagccag

SEQ ID NO: 91 - DNA, 21 bp, Homo sapiens
attttgtcccgagaaggtggc

SEQ ID NO: 92 - DNA, 23 bp, Homo sapiens
agcaggcaaggattatggttctc

SEQ ID NO: 93 - DNA, 22 bp, Homo sapiens
ttctcaagggtgcgagctcatc

SEQ ID NO: 94 - DNA, 20 bp, Homo sapiens
tcctcccttggccaccaatg

SEQ ID NO: 95 - DNA, 23 bp, Homo sapiens
aaggggtcactatggagttcaaa

SEQ ID NO: 96 - DNA, 23 bp, Homo sapiens
ggcactcatattctccagcatct

SEQ ID NO: 97 - DNA, 23 bp, Homo sapiens
tgggctattggattaagtgtggc

SEQ ID NO: 98 - DNA, 21 bp, Homo sapiens
ttgacacgggatgaatcctgg

SEQ ID NO: 99 - DNA, 22 bp, Homo sapiens
tggggagaatcacatttgggtc

SEQ ID NO: 100 - DNA, 22 bp, Homo sapiens
atggcttcagagctggtcatgg

SEQ ID NO: 101 - DNA, 23 bp, Homo sapiens
tcgcagatgaattcagagcacag

SEQ ID NO: 102 - DNA, 23 bp, Homo sapiens
ttgatgcgtgcacagatcttcag

SEQ ID NO: 103 - DNA, 17 bp, Homo sapiens
gcaccacccgctttttc

SEQ ID NO: 104 - DNA, 22 bp, Homo sapiens
gatccttgtcgatatccttctg

SEQ ID NO: 105 - DNA, 22 bp, Homo sapiens
cattcagggtctatttgctccg

SEQ ID NO: 106 - DNA, 20 bp, Homo sapiens
gaagacgctgaacctgctgc

SEQ ID NO: 107 - DNA, 23 bp, Homo sapiens
acacactcagtgcagcaatatat

SEQ ID NO: 108 - DNA, 23 bp, Homo sapiens
ggagtgtcggttgttaagaacta

SEQ ID NO: 109 - DNA, 219 bp, Homo sapiens
ggagggcagattcggaccactaggcctgaaatgacatttcactaaaagtctccaaaacatttctaagactactaaggccttttatgtaattcctttaaatgtgtatttcttaagaattcaaatttgtaataaaactatttgtataaaaattaagcttttattaatttgttgctagtattgccacagacgcattaaaagaaacttactgcacaagctgct